SEQUENCE LISTING (290 sequences)

SEQ ID NO: 1 - protein, 241 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYIQGLGYYFDYWGQGTLVTVSTGGGGSGGGGSGGGGSSYELMQPPSVSVSPGQTASITCSGDKLGDKYVSWYQQKPGQSPVLVIYQDTKRPSGIPERFSGSNSGNTATLTISGTQAMDEADYYCQAWDSGTAIFGGGTKVTVL

SEQ ID NO: 2 - protein, 241 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYIQGLGYYFDYWGQGTLVTVSTGGGGSGGGGSGGGGSQAGLTQPPSVSVSPGQTASITCSGDKLGDKYVSWYQQKPGQSPVLVIYQDSKRPSGIPERFSGSNSGNTATLTISGTQAMDEADYYCQAWDSSTVVFGGGTKVTVL

SEQ ID NO: 3 - protein, 241 aa, Homo sapiens
QLQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYIQGLGYYFDYWGQGTLVTVSTGGGGSGGGGSGGGGSQAGLTQPPSVSVSPGQTASITCFGDKLGHKYVSWYQQKPGQSPVLVIYQDSKRPSGIPERFSGSNSGNTATLTISGTQAMDEADYYCQAWDSSTVVFGGGTKLTVL

SEQ ID NO: 4 - protein, 243 aa, Homo sapiens
QMQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKPSRGYSRSLDYWGQGTLVTVSTGGGGSGGGGSGGGGSQPVLTQPPSVSVSPGQTASITCSGDKLGDKFTSWYQQRPGQSPVLVIYQDNKRPSGIPERFSGSNSGNTATLTISRVEAGDEADYYCQVWDSSSDHWVFGGGTQLTVL

SEQ ID NO: 5 - protein, 241 aa, Homo sapiens
QVQLVESGAEVKKPGESLKISCKGSGYSFTSYWIGWVRQMPGKGLEWMGIIYPGDSDTRYSPSFQGQVTISADKSISTAYLQWSSLKASDTAMYYCARLQSGWLHAFDIWGQGTMVTVSTGGGGSGGGGSGGGGSQSVLTQPPSVSVSPGQTARISCSGDKLGDKYVSWYQQKPGQSPVLVIYEDSKRPSGIPERLSGSNSGNTATLTISGTQAMDEADYYCQAWDSSTVVFGGGTKLTVL

SEQ ID NO: 6 - protein, 249 aa, Homo sapiens
QVQLLQSAAEVKKPGESLKISCKGSGYSFTSYWIGWVRQMPGKGLEWMGIIYPGDSDTRYSPSFQGQITISADKSISTAYLQWSSLKASDTAMYYCARLKWSGLSHYYYYYMDVWGKGTTVTVSTGGGGSGGGGSGGGGSLSELTQDPAVSVALGQTVRITCQGDSLRNYYASWYQQKPGQAPVLVIYGKNNRPSGIPDRFSGSRSGNTASLTITGAQAEDEADYYCNSRDSSGNHPVVFGGGTKLTVL

SEQ ID NO: 7 - protein, 248 aa, Homo sapiens
QVQLLQSAAEVKKPGESLKISCKGSGYSFTSYWIGWVRQMPGKGLEWMGIIYPGDSDTRYSPSFQGQVTISADKSISTAYLQWSSLKASDTAMYYCARLKWSGLSHYYYYYMDVWGKGTTVTVSTGGGGSGGGGSGGGGSLSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTKLTVL

SEQ ID NO: 8 - protein, 248 aa, Homo sapiens
QVQLLQSAAEVKKPGESLKISCKGSGYSFTSYWIGWVRQMPGKGLEWMGIIYPGDSDTRYSPSFQGQVTISADKSISTAYLQWSSLKASDTAMYYCARLKWSGLSHYYYYYMDVWGKGTTVTVSTGGGGSGGGGSGGGGSLSELTQDPAVSVALGQTVRITCQGDSLRSYYASWYQQKPGQAPVLVIYGKNNRPSGIPDRFSGSSSGNTASLTITGAQAEDEADYYCNSRDSSGNHVIFGGGTKLTVL

SEQ ID NO: 9 - protein, 243 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLPLGLQVGFDYWGQGTLVTVSTGGGGSGGGGSGGGGSLPVLTQPPSVSVSPGQTASITCSGDKLGDKYASWYQQKPGQSPVLIIYQDTKRASGIPERFSGSNSGNTATLTISGTQAVDEADYYCQAFDSSAAHFVFGAGTKLTVL

SEQ ID NO: 10 - protein, 241 aa, Homo sapiens
QMQLVQSGAEVKKPGESLKISCKGSGYSFTSYWIGWVRQMPGKGLEWMGIIYPGDSDTRYSPSFQGQVTISADKSISTAYLQWSSLKASDTAMYYCARVRYSYDLNFDYWGQGTLVTVSTGGGGSGGGGSGGGGSSYELMQPPSVSVSPGQTASITCSGDKLGDKYASWYQQKPGQSPVLVIYQDNKRPSGIPERFSGSNSGNTATLTISGTQAMDEADYYCQTWDSSTAVFGGGTKVTVL

SEQ ID NO: 11 - protein, 241 aa, Mus musculus
QVQLQQSGAELAKPGASVKLSCKTSGYTFTNFWMHWVKQRPGQGLEWIGYINPSSDYTKYNQKFKGKATLTADKSSSTAYMQLSSLTYEDSAVYYCARDDYSDFGFAYWGQGTLVTVSAGGGGSGGGGSGGGGSDIQMTQSPASLSASVGETVTITCRASENIYSFLAWYQQKQGKSPQLLVYNAKTLAEGVPSRFSGSGSGTQFSLKINSLQPEDFGSYYCQHHYGIPPTFGGGTKLEIK

SEQ ID NO: 12 - protein, 239 aa, Mus musculus
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCAREANWDDVDYWGQGTTLTVSSGGGGSGGGGSGGGGSQIVLTQSPAIMSASPGEKVTMTCSASSSVSYMYWYLQKPGSSPRLLIYDTSNLASGVPVRFSGSGSGTSYSLTISRMEAEDAATYYCQQWSSYPLTFGAGTKLELK

SEQ ID NO: 13 - protein, 245 aa, Mus musculus
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCAREANWDDVDYWGQGTTLTVSSGGGGSGGGGSGGGGSDVLMTQTPLSLPVSLGDQASISCRSSQSIVHSNGNTYLEWYLQKPGQSPKLLIYKVSNRFSGVPDRFSGSGSGTDFTLKISRVEAEDLGLYYCFQGSHVPYTFGGGTKLEIK

SEQ ID NO: 14 - protein, 248 aa, Mus musculus
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARNYYSSSYDGYFDYWGQGTTLTVSSGGGGSGGGGSGGGGSDIVLTQSPASLAVSLGQRATISCRASESVDNYGISFMHWYQQKPGQPPKFLIYRASNLESGIPARFSGSGSRTDFTLTINPVETDDVATYYCQQSNKDPRTFGGGTKLEIK

SEQ ID NO: 15 - protein, 239 aa, Mus musculus
QVQLQQSGPELVKPGASVKISCRASGYTFTDYYIDWVKQRPGQGLEWIGWIFPGTNSTYYNEKFKGKATLTVDKSSSTAYMLLSSLTSEDSAVYFCARSGLRDFDYWGQGTTLTVSSGGGGSGGGGSGGGGSDIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 16 - protein, 245 aa, Mus musculus
EVQLQQSGPVLVKPGASVKMSCKASGYTFTDYYMNWVKQSHGKGLEWIAVINPYSGGTSYNQKFKGKATLTVDKSSSTAYMELSSLTSEDSAVYYCASVSSYGNYFDYWGQGTTLTVSSGGGGSGGGGSGGGGSDIVLTQSPASLAVSLGQRATISCRASESVSIHASHLLHWYQQKPGQPPKLLIYAASNLESGVPARFSGSGSETDFTLNIHPVEEEDAATYFCQQSIEDPWTFGGGTKLEIK

SEQ ID NO: 17 - protein, 242 aa, Mus musculus
QVQLQQSGPELVKPGASVKISCKASGYTFTDYYINWVKQRPGQGLEWIGWIFPGSGSTYYNEKFKGKATLTVDKSSSTVYMLLSSLTSEDSAVYFCAREAKLGRDYFDYWGQGTTLTVSSGGGGSGGGGSGGGGSDIVMTQSHKFMSTSVGDRVSITCKASQDVSTAVAWCQQKPGQSPKLLIYSASYRYTGVPDRFTGSGSGTDFTFTISSVQAEDLAVYYCQQHYSTPYTFGGGTRLEIK

SEQ ID NO: 18 - protein, 248 aa, Mus musculus
EFQLQQSGPELVKPGASVKISCKASGYSFTDYNMNWVKQSNGKSLAWIGVINPNYGTTNYNQKFKGKATLTVDQSSSTAYMQLNSLTSEDSAVYYCARNYYGSTYDGYFDYWGQGTTLTVSSGGGGSGGGGSGGGGSDIVLTQSPASLAVSLGQRATISCRASESVDNYGISFMHWYQQKPGQPPKFLIYRASNLESGIPARFSGSGSRTDFTLTINPVETDDVATYYCQQSNKDPRTFGGGTKLEIT

SEQ ID NO: 19 - protein, 248 aa, Mus musculus; identical to SEQ ID NO: 18

SEQ ID NO: 20 - protein, 239 aa, Mus musculus
QVQLQQSGAELMKPGASVKISCKATGYTINGYWIEWVKERPGHGLEWIGEILPGSGSTNYNEKFKGKATFTADTSSNTAYMQLSSLTTEDSAIYYCARGMEGAMDYWGQGTSVTVSSGGGGSGGGGSGGGGSDIVMTQSHKFMSTSVGDRVSITCKASQDVSTAVAWYQKKPGQSPKLLIYSASYRYTGVPDRFTGSGSGTDFTFTISSVQAEDLAVYYCQQHYSTPPTFGGGTKLEIK

SEQ ID NO: 21 - protein, 244 aa, Mus musculus
QVQLQQPGAELVMPGASVRLSCKASGYTFTSYWMHWVKQRPGQGLEWIGEIDPSESYPNYNQNFKGKATLTVDKSSSTAYMQLSSLTSEDSAVYYCARSYYGRSGYAMDYWGQGTSVTVSSGGGGSGGGGSGGGGSNIVMTQSPKSTSMSVGERVTLNCKASENVGTYVSWYQQKPEQSPKLLIYGASNRYTGVPDRFTGSGSATDFTLTISSVQAEDLADYHCGQSYSYPPFTFGSGTKLEIK

SEQ ID NO: 22 - protein, 244 aa, Mus musculus
QVQLTESGPGLVAPSQSLSITCTVSGFSLTNYIISWVRQPPGKGLEWLGVIWTGGGTNYNSALKSRLSISKDDSKSQVFLKMNSLQTDDTARYYCARNEAVVAIFDWYFDVWGTGTTVTVSSGGGGSGGGGSGGGGSDIQMTQTTSSLSASLGDRVTISCRASQDISNYLNWYQQKPDGAVKLLIYYTSRLHSGVPSRFSGSGSGTDFSLTISNLEQEDFATYFCQQGNTLPWTFGGGTKLEIK

SEQ ID NO: 23 - protein, 248 aa, Mus musculus
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARNYYGNNYDGYFDYWGQGTTLTVSSGGGGSGGGGSGGGGSDIVLTQSPASLAVSLGQRATISCRASESVDNYGISFMHWYQQKPGQPPKFLIYRASNLESGIPARFSGSGSRTDFTLTINPVETDDVATYYCQQSNKDPRTFGGGTKLEIT

SEQ ID NO: 24 - protein, 242 aa, Mus musculus
QIQLVQSGPELKKPGETVKISCKASGYTFTMYGMSWVKQAPGKGLKWMGWINTYSGVPTYADDFKGRFAFSLETSANTAYLQINNLKNEDTATYFCARFPYDYDGYFDVWGTGTTVTVSSGGGGSGGGGSGGGGSDIVMTQSHKFMSTSVGDRVSITCKASQDVGTAVAWYQQKPGQSPKLLIYWASTRHTGVPDRFTGSGSGTDFTLAISNVQSEDLADYFCHQFSSYPLTFGAGTRLELK

SEQ ID NO: 25 - protein, 248 aa, Mus musculus
EVQLQQSGPELVKPGASVKISCKASGYTFTDYYMNWVKQSHGKSLEWIGDINPNNGGTTYNQKFKGKATLTVDKSSSTAYMELRSLTSEDSAVYYCARRYYSSGYDGYFDVWGTGTTVTVSSGGGGSGGGGSGGGGSDIVLTQSPASLAVSLGQRATISCRASENVDNYGISFMHWYQQKPGQPPKFLIYRASNLEYGIPARFSGSGSRTDFTLTINPVETDDVATYYCQQSNKDPLTFGAGTKLELK

SEQ ID NO: 26 - protein, 244 aa, Mus musculus
EVQLQQSGPELVKPGASVKMSCKASGSTFTSYVMHWVKQKPGQGLEWIGYSNPYNDGTKYNEKFKGKATLTSDKSSSTAYMELSSLTSEDSAVYYCARLNVLYYFDNWGQGTTLTVSSGGGGSGGGGSGGGGSDIVLTQSPASLAVSLGQRATISCRASKSVSTSGYTYMHWYQQKPGQPPKLLIYLASNLESGVPARFSGSGSGTDFTLNIHPVEEEDAATYYCQHSRELPLTFGAGTKLELK

SEQ ID NO: 27 - protein, 251 aa, Mus musculus
QVQLQQPGTELVKPGASVKLSCKASGYTFTSYWMHWVKQRPGQGLEWIGNINPGNGGTDYNEKIKSKATLTVDKSTSTAYMQLSSLTSEDSAVYYCARGGGYYGYDGYWYFDVWGTGTTVTVSSGGGGSGGGGSGGGGSDIVMTQAAFSIPVTLGTSTSISCRSTKSLLHSNGITYLYWYLQKPGQSPQLLIYQMSNLASGVPDRFSSSGSGTDFTLRISRVEAEDVGVYYCAQNLELPWTFGGGTKLEIK

SEQ ID NO: 28 - protein, 243 aa, Mus musculus
EVQLQQSGPVLVKPGPSVKISCEASGFTFTDYYMHWVKQNHGKSLEWIGLVYPYNGDTIYNQKFKGKATLTVDTSSSTAYMDLHSLTSEDSAVYYCARGANWGDYWGQGTTLTVSSGGGGSGGGGSGGGGSDVVMTQTPLSLPVSLGDQASISCRSSQSLVHSNGNTYLHWFLQKPGQSPKLLIYKVSNRFSGVPDRFSGSGSGTDFTLKISRVEAEDLGLYFCSQSTHVPPTFGGGTKLEIK

SEQ ID NO: 29 - protein, 241 aa, Mus musculus
QVQLQQPGTELVKPGASVKLSCKASGYTFTSYWMHWVKQRPGQGLEWIGNISPSNGGTNYNENFKSKATLTVDKSSSTAYMQLSSLTSEDSAVYYCATYYVDYWGQGTTLTVSSGGGGSGGGGSGGGGSDVVMTQTPLTLSVTIGQPASISCKSTQSLLDSDGKTYLNWFLQRPGQSPKRLIYLVSKLDSGVPDRFTGSGSGTDFTLKISRVEAEDLGLYYCWQGTHFPQTFGGGTKLEIK

SEQ ID NO: 30 - protein, 243 aa, Mus musculus
QVTLKESGPGILQSSQTLSLTCSFSGFSLSTSGMGVSWIRQPSGKGLEWLAHIYWDDDKRYNPSLKSRLTISKDTSRNQVFLKITSVDTADTATYYCARRVYGYDPYAMNYWGPGTSVTVSSGGGGSGGGGSGGGGSQIVLTQSPALMSASPGEKVTMTCSASSSVSYMYWYQQKPRSSPKPWIYLTSTLASGVPARFSGSGSGTSYSLTISSMEAEDAATYYCQQWSSNPYTFGGGTKLEIK

SEQ ID NO: 31 - protein, 248 aa, Mus musculus
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCAPHYYGSSYDWYFDVWGTGTTVTVSSGGGGSGGGGSGGGGSDIVLTQSPASLAVSLGQRATISCRASESVDNYGISFMHWYQQKPGQPPKLLIYRASNLESGIPARFSGSGSRTDFTLTINPVETDDVATYYCQQSNKDPLTFGAGTKLELK

SEQ ID NO: 32 - protein, 242 aa, Mus musculus
EVQLQQSGPELVKPGASVKIPCKASGYTFTDYNMDWVKQSHGKSLEWIGDINPNNGGTIYNQKFKGKATLTVDKSSSTAYMELRSLTSEDTAVYYCAREDDSRYWYFDVWGTGTTVTVSSGGGGSGGGGSGGGGSDIVMTQSHKFMSTSVGDRVSITCKASQDVGTAVAWYQQKPGQSPKLLIYWASTRHTGVPDRFTGSGSGTDFTLTISNVQSEDLADYFCQQYSSYPYTFGGGTKLEIK

SEQ ID NO: 33 - protein, 240 aa, Mus musculus
QIQFVQSGPELKKPGETVKISCKASVYTFTEYPMHWVKQAPGKGFKWMGWINTYSGEPTYADDFKGRFAFSLETSASTAYLQINNLKNEDTASYFCAREGWLDAMDYWGQGTSVTVSSGGGGSGGGGSGGGGSDIVMTQSQKFMSTIVGDRVSITCKASQNVGTAVAWYQQKPGQSPKLLIYSASNRYTGVPDRFTGSGSGTDFTLTISNMQSEDLADYFCQQYSSYTWTFGGGTKLEIK

SEQ ID NO: 34 - protein, 237 aa, Mus musculus
QVQLKQSGPGLVQPSQSLSITCTVSGFSLTTFGVHWVRQSPGKGLEWLGVIWSGGSTDYNAAFISRLSISKDNSKSQVFFKMNSLQVDDTAIYYCAPRLGLRAYWGQGTLVTVSAGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEVK

SEQ ID NO: 35 - protein, 239 aa, Mus musculus
QVQLKQSGPGLVQPSQSLSITCTVSGFSLTSYGVHWVRQSPGKGLEWLGVIWSGGTTDYNAAFISRLSISKDNSKSQVFFKMNSLQADDTAIYYCARMGGTGYFDVWGTGTTVTVSSGGGGSGGGGSGGGGSNIVMTQSPKSMSMSVGERVTLSCKAGENVGPYVSWYQQKPEQSPKLLIYGASNRFTGVPDRFTGSGSATDFTLTISSVQAEDLADYHCGQSYSYPFTFGSGTKLEIK

SEQ ID NO: 36 - protein, 244 aa, Mus musculus
QVQLKQSGPGLVQPSQSLSITCTVSGFSLTSYGVHWVRQSPGKGLEWLGVIWSGGTTDYNAAFISRLSISKDNSKSQVFFKMNSLQADDTAIYYCARMGGTGYFDVWGTGTTVTVSSGGGGSGGGGSGGGGSDIVMTQAAFSNPVTLGTSASISCRSSKSLLHSNGITYLYWYLQKPGLSPQLLIYHMSNLASGVPDRFSSSGSGTDFTLRISRVEAEDVGVYYCAQNLELPWTFGGGTKLEIK

SEQ ID NO: 37 - protein, 238 aa, Mus musculus
QVQLVQSGAEVKKPGASVKVSCKASGYTFTDYNMHWVRQAPGQGLEWMGYIYPYNGGTAYNQKFKNRVTMTRDTSTSTVYMELSSLRSEDTAVYYCARGLDVMDYWGQGTLVTVSSGGGGSGGGGSGGGGSAIQLTQSPSSLSASVGDRVTITCRASENIYSYLAWYQQKPGKAPKLLIYNAKILAEGVPSRFSGSGSGTDFTLTISSLQPEDFATYYCQHHYVSPRTFGQGTKLEIK

SEQ ID NO: 38 - protein, 238 aa, Mus musculus
DIQMTQSPSSLSASVGDRVTITCRASENIYSYLAWYQQKPGKAPKLLIYNAKILAEGVPSRFSGSGSGTDFTLTISSLQPEDFATYYCQHHYVSPRTFGQGTKLEIKGGGGSGGGGSGGGGSQVQLVQSGAEVKKPGASVKVSCKVSGYTFTDYNMHWVRQAPGKGLEWMGYIYPYNGGTAYNQKFKNRVTMTEDTSTDTAYMELSSLRSEDTAVYYCARGLDVMDYWGQGTLVTVSS

SEQ ID NO: 39 - protein, 120 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYIQGLGYYFDYWGQGTLVTVST

SEQ ID NO: 40 - protein, 120 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYIQGLGYYFDYWGQGTLVTVST

SEQ ID NO: 41 - protein, 120 aa, Homo sapiens
QLQLVQSGAEVKKPGESLKISCKGSGYSFTSYWIGWVRQMPGKGLEWMGIIYPGDSDTRYSPSFQGQVTISADKSISTAYLQWSSLKASDTAMYYCARYIQGLGYYFDYWGQGTLVTVST

SEQ ID NO: 42 - protein, 120 aa, Homo sapiens
QMQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKPSRGYSRSLDYWGQGTLVTVST

SEQ ID NO: 43 - protein, 120 aa, Homo sapiens
QVQLVESGAEVKKPGESLKISCKGSGYSFTSYWIGWVRQMPGKGLEWMGIIYPGDSDTRYSPSFQGQVTISADKSISTAYLQWSSLKASDTAMYYCARLQSGWLHAFDIWGQGTMVTVST

SEQ ID NO: 44 - protein, 125 aa, Homo sapiens
QVQLLQSAAEVKKPGESLKISCKGSGYSFTSYWIGWVRQMPGKGLEWMGIIYPGDSDTRYSPSFQGQITISADKSISTAYLQWSSLKASDTAMYYCARLKWSGLSHYYYYYMDVWGKGTTVTVST

SEQ ID NO: 45 - protein, 125 aa, Homo sapiens
QVQLLQSAAEVKKPGESLKISCKGSGYSFTSYWIGWVRQMPGKGLEWMGIIYPGDSDTRYSPSFQGQVTISADKSISTAYLQWSSLKASDTAMYYCARLKWSGLSHYYYYYMDVWGKGTTVTVST

SEQ ID NO: 46 - protein, 125 aa, Homo sapiens; identical to SEQ ID NO: 45

SEQ ID NO: 47 - protein, 120 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLPLGLQVGFDYWGQGTLVTVST

SEQ ID NO: 48 - protein, 120 aa, Homo sapiens
QMQLVQSGAEVKKPGESLKISCKGSGYSFTSYWIGWVRQMPGKGLEWMGIIYPGDSDTRYSPSFQGQVTISADKSISTAYLQWSSLKASDTAMYYCARVRYSYDLNFDYWGQGTLVTVST

SEQ ID NO: 49 - protein, 119 aa, Mus musculus
QVQLQQSGAELAKPGASVKLSCKTSGYTFTNFWMHWVKQRPGQGLEWIGYINPSSDYTKYNQKFKGKATLTADKSSSTAYMQLSSLTYEDSAVYYCARDDYSDFGFAYWGQGTLVTVSA

SEQ ID NO: 50 - protein, 118 aa, Mus musculus
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCAREANWDDVDYWGQGTTLTVSS

SEQ ID NO: 51 - protein, 118 aa, Mus musculus; identical to SEQ ID NO: 50

SEQ ID NO: 52 - protein, 122 aa, Mus musculus
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARNYYSSSYDGYFDYWGQGTTLTVSS

SEQ ID NO: 53 - protein, 117 aa, Mus musculus
QVQLQQSGPELVKPGASVKISCRASGYTFTDYYIDWVKQRPGQGLEWIGWIFPGTNSTYYNEKFKGKATLTVDKSSSTAYMLLSSLTSEDSAVYFCARSGLRDFDYWGQGTTLTVSS

SEQ ID NO: 54 - protein, 119 aa, Mus musculus
EVQLQQSGPVLVKPGASVKMSCKASGYTFTDYYMNWVKQSHGKGLEWIAVINPYSGGTSYNQKFKGKATLTVDKSSSTAYMELSSLTSEDSAVYYCASVSSYGNYFDYWGQGTTLTVSS

SEQ ID NO: 55 - protein, 120 aa, Mus musculus
QVQLQQSGPELVKPGASVKISCKASGYTFTDYYINWVKQRPGQGLEWIGWIFPGSGSTYYNEKFKGKATLTVDKSSSTVYMLLSSLTSEDSAVYFCAREAKLGRDYFDYWGQGTTLTVSS

SEQ ID NO: 56 - protein, 120 aa, Mus musculus
QIQLVQSGPELKKPGETVKISCKASGYTFTTYGMSWVKQAPGKGLKWMGWINTYSGVPTYADDFKGRFAFSLETSASTAYLQINNLKNEDTTTYFCARFPYDFDGYFDVWGTGTAVTVSS

SEQ ID NO: 57 - protein, 122 aa, Mus musculus
EFQLQQSGPELVKPGASVKISCKASGYSFTDYNMNWVKQSNGKSLAWIGVINPNYGTTNYNQKFKGKATLTVDQSSSTAYMQLNSLTSEDSAVYYCARNYYGSTYDGYFDYWGQGTTLTVSS

SEQ ID NO: 58 - protein, 117 aa, Mus musculus
QVQLQQSGAELMKPGASVKISCKATGYTINGYWIEWVKERPGHGLEWIGEILPGSGSTNYNEKFKGKATFTADTSSNTAYMQLSSLTTEDSAIYYCARGMEGAMDYWGQGTSVTVSS

SEQ ID NO: 59 - protein, 121 aa, Mus musculus
QVQLQQPGAELVMPGASVRLSCKASGYTFTSYWMHWVKQRPGQGLEWIGEIDPSESYPNYNQNFKGKATLTVDKSSSTAYMQLSSLTSEDSAVYYCARSYYGRSGYAMDYWGQGTSVTVSS

SEQ ID NO: 60 - protein, 122 aa, Mus musculus
QVQLTESGPGLVAPSQSLSITCTVSGFSLTNYIISWVRQPPGKGLEWLGVIWTGGGTNYNSALKSRLSISKDDSKSQVFLKMNSLQTDDTARYYCARNEAVVAIFDWYFDVWGTGTTVTVSS

SEQ ID NO: 61 - protein, 122 aa, Mus musculus
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARNYYGNNYDGYFDYWGQGTTLTVSS

SEQ ID NO: 62 - protein, 120 aa, Mus musculus
QIQLVQSGPELKKPGETVKISCKASGYTFTMYGMSWVKQAPGKGLKWMGWINTYSGVPTYADDFKGRFAFSLETSANTAYLQINNLKNEDTATYFCARFPYDYDGYFDVWGTGTTVTVSS

SEQ ID NO: 63 - protein, 122 aa, Mus musculus
EVQLQQSGPELVKPGASVKISCKASGYTFTDYYMNWVKQSHGKSLEWIGDINPNNGGTTYNQKFKGKATLTVDKSSSTAYMELRSLTSEDSAVYYCARRYYSSGYDGYFDVWGTGTTVTVSS

SEQ ID NO: 64 - protein, 118 aa, Mus musculus
EVQLQQSGPELVKPGASVKMSCKASGSTFTSYVMHWVKQKPGQGLEWIGYSNPYNDGTKYNEKFKGKATLTSDKSSSTAYMELSSLTSEDSAVYYCARLNVLYYFDNWGQGTTLTVSS

SEQ ID NO: 65 - protein, 124 aa, Mus musculus
QVQLQQPGTELVKPGASVKLSCKASGYTFTSYWMHWVKQRPGQGLEWIGNINPGNGGTDYNEKIKSKATLTVDKSTSTAYMQLSSLTSEDSAVYYCARGGGYYGYDGYWYFDVWGTGTTVTVSS

SEQ ID NO: 66 - protein, 116 aa, Mus musculus
EVQLQQSGPVLVKPGPSVKISCEASGFTFTDYYMHWVKQNHGKSLEWIGLVYPYNGDTIYNQKFKGKATLTVDTSSSTAYMDLHSLTSEDSAVYYCARGANWGDYWGQGTTLTVSS

SEQ ID NO: 67 - protein, 114 aa, Mus musculus
QVQLQQPGTELVKPGASVKLSCKASGYTFTSYWMHWVKQRPGQGLEWIGNISPSNGGTNYNENFKSKATLTVDKSSSTAYMQLSSLTSEDSAVYYCATYYVDYWGQGTTLTVSS

SEQ ID NO: 68 - protein, 122 aa, Mus musculus
QVTLKESGPGILQSSQTLSLTCSFSGFSLSTSGMGVSWIRQPSGKGLEWLAHIYWDDDKRYNPSLKSRLTISKDTSRNQVFLKITSVDTADTATYYCARRVYGYDPYAMNYWGPGTSVTVSS

SEQ ID NO: 69 - protein, 122 aa, Mus musculus
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCAPHYYGSSYDWYFDVWGTGTTVTVSS

SEQ ID NO: 70 - protein, 120 aa, Mus musculus
EVQLQQSGPELVKPGASVKIPCKASGYTFTDYNMDWVKQSHGKSLEWIGDINPNNGGTIYNQKFKGKATLTVDKSSSTAYMELRSLTSEDTAVYYCAREDDSRYWYFDVWGTGTTVTVSS

SEQ ID NO: 71 - protein, 118 aa, Mus musculus
QIQFVQSGPELKKPGETVKISCKASVYTFTEYPMHWVKQAPGKGFKWMGWINTYSGEPTYADDFKGRFAFSLETSASTAYLQINNLKNEDTASYFCAREGWLDAMDYWGQGTSVTVSS

SEQ ID NO: 72 - protein, 115 aa, Mus musculus
QVQLKQSGPGLVQPSQSLSITCTVSGFSLTTFGVHWVRQSPGKGLEWLGVIWSGGSTDYNAAFISRLSISKDNSKSQVFFKMNSLQVDDTAIYYCAPRLGLRAYWGQGTLVTVSA

SEQ ID NO: 73 - protein, 117 aa, Mus musculus
QVQLKQSGPGLVQPSQSLSITCTVSGFSLTSYGVHWVRQSPGKGLEWLGVIWSGGTTDYNAAFISRLSISKDNSKSQVFFKMNSLQADDTAIYYCARMGGTGYFDVWGTGTTVTVSS

SEQ ID NO: 74 - protein, 117 aa, Mus musculus; identical to SEQ ID NO: 73

SEQ ID NO: 75 - protein, 116 aa, Mus musculus
QVQLVQSGAEVKKPGASVKVSCKASGYTFTDYNMHWVRQAPGQGLEWMGYIYPYNGGTAYNQKFKNRVTMTRDTSTSTVYMELSSLRSEDTAVYYCARGLDVMDYWGQGTLVTVSS

SEQ ID NO: 76 - protein, 116 aa, Mus musculus
QVQLVQSGAEVKKPGASVKVSCKVSGYTFTDYNMHWVRQAPGKGLEWMGYIYPYNGGTAYNQKFKNRVTMTEDTSTDTAYMELSSLRSEDTAVYYCARGLDVMDYWGQGTLVTVSS

SEQ ID NO: 77 - protein, 106 aa, Homo sapiens
SYELMQPPSVSVSPGQTASITCSGDKLGDKYVSWYQQKPGQSPVLVIYQDTKRPSGIPERFSGSNSGNTATLTISGTQAMDEADYYCQAWDSGTAIFGGGTKVTVL

SEQ ID NO: 78 - protein, 106 aa, Homo sapiens
QAGLTQPPSVSVSPGQTASITCSGDKLGDKYVSWYQQKPGQSPVLVIYQDSKRPSGIPERFSGSNSGNTATLTISGTQAMDEADYYCQAWDSSTVVFGGGTKVTVL

SEQ ID NO: 79 - protein, 106 aa, Homo sapiens
QAGLTQPPSVSVSPGQTASITCFGDKLGHKYVSWYQQKPGQSPVLVIYQDSKRPSGIPERFSGSNSGNTATLTISGTQAMDEADYYCQAWDSSTVVFGGGTKLTVL

SEQ ID NO: 80 - protein, 108 aa, Homo sapiens
QPVLTQPPSVSVSPGQTASITCSGDKLGDKFTSWYQQRPGQSPVLVIYQDNKRPSGIPERFSGSNSGNTATLTISRVEAGDEADYYCQVWDSSSDHWVFGGGTQLTVL

SEQ ID NO: 81 - protein, 106 aa, Homo sapiens
QSVLTQPPSVSVSPGQTARISCSGDKLGDKYVSWYQQKPGQSPVLVIYEDSKRPSGIPERLSGSNSGNTATLTISGTQAMDEADYYCQAWDSSTVVFGGGTKLTVL

SEQ ID NO: 82 - protein, 109 aa, Homo sapiens
LSELTQDPAVSVALGQTVRITCQGDSLRNYYASWYQQKPGQAPVLVIYGKNNRPSGIPDRFSGSRSGNTASLTITGAQAEDEADYYCNSRDSSGNHPVVFGGGTKLTVL

SEQ ID NO: 83 - protein, 108 aa, Homo sapiens
LSELTQDPAVSVALGQTVRITCQGDSLRSYYASWYQQKPGQAPVLVIYGKNNRPSGIPDRFSGSSSGNTASLTITGAQAEDEADYYCNSRDSSGNHLVFGGGTKLTVL

SEQ ID NO: 84 - protein, 108 aa, Homo sapiens
LSELTQDPAVSVALGQTVRITCQGDSLRSYYASWYQQKPGQAPVLVIYGKNNRPSGIPDRFSGSSSGNTASLTITGAQAEDEADYYCNSRDSSGNHVIFGGGTKLTVL

SEQ ID NO: 85 - protein, 108 aa, Homo sapiens
LPVLTQPPSVSVSPGQTASITCSGDKLGDKYASWYQQKPGQSPVLIIYQDTKRASGIPERFSGSNSGNTATLTISGTQAVDEADYYCQAFDSSAAHFVFGAGTKLTVL

SEQ ID NO: 86 - protein, 106 aa, Homo sapiens
SYELMQPPSVSVSPGQTASITCSGDKLGDKYASWYQQKPGQSPVLVIYQDNKRPSGIPERFSGSNSGNTATLTISGTQAMDEADYYCQTWDSSTAVFGGGTKVTVL

SEQ ID NO: 87 - protein, 107 aa, Mus musculus
DIQMTQSPASLSASVGETVTITCRASENIYSFLAWYQQKQGKSPQLLVYNAKTLAEGVPSRFSGSGSGTQFSLKINSLQPEDFGSYYCQHHYGIPPTFGGGTKLEIK

SEQ ID NO: 88 - protein, 106 aa, Mus musculus
QIVLTQSPAIMSASPGEKVTMTCSASSSVSYMYWYLQKPGSSPRLLIYDTSNLASGVPVRFSGSGSGTSYSLTISRMEAEDAATYYCQQWSSYPLTFGAGTKLELK

SEQ ID NO: 89 - protein, 112 aa, Mus musculus
DVLMTQTPLSLPVSLGDQASISCRSSQSIVHSNGNTYLEWYLQKPGQSPKLLIYKVSNRFSGVPDRFSGSGSGTDFTLKISRVEAEDLGLYYCFQGSHVPYTFGGGTKLEIK

SEQ ID NO: 90 - protein, 111 aa, Mus musculus
DIVLTQSPASLAVSLGQRATISCRASESVDNYGISFMHWYQQKPGQPPKFLIYRASNLESGIPARFSGSGSRTDFTLTINPVETDDVATYYCQQSNKDPRTFGGGTKLEIK

SEQ ID NO: 91 - protein, 107 aa, Mus musculus
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 92 - protein, 111 aa, Mus musculus
DIVLTQSPASLAVSLGQRATISCRASESVSIHASHLLHWYQQKPGQPPKLLIYAASNLESGVPARFSGSGSETDFTLNIHPVEEEDAATYFCQQSIEDPWTFGGGTKLEIK

SEQ ID NO: 93 - protein, 107 aa, Mus musculus
DIVMTQSHKFMSTSVGDRVSITCKASQDVSTAVAWCQQKPGQSPKLLIYSASYRYTGVPDRFTGSGSGTDFTFTISSVQAEDLAVYYCQQHYSTPYTFGGGTRLEIK

SEQ ID NO: 94 - protein, 107 aa, Mus musculus
DIVMTQSHKFMSTSVGDRVSITCKASQDVGTAVAWYQEKPGQSPKILIYWASTRHTGVPDRFTGSGSGTDFTLTISNVQSEDLADYFCQQYSSYPLTFGAGTKLELK

SEQ ID NO: 95 - protein, 111 aa, Mus musculus
DIVLTQSPASLAVSLGQRATISCRASESVDNYGISFMHWYQQKPGQPPKFLIYRASNLESGIPARFSGSGSRTDFTLTINPVETDDVATYYCQQSNKDPRTFGGGTKLEIT

SEQ ID NO: 96 - protein, 107 aa, Mus musculus
DIVMTQSHKFMSTSVGDRVSITCKASQDVSTAVAWYQKKPGQSPKLLIYSASYRYTGVPDRFTGSGSGTDFTFTISSVQAEDLAVYYCQQHYSTPPTFGGGTKLEIK

SEQ ID NO: 97 - protein, 108 aa, Mus musculus
NIVMTQSPKSTSMSVGERVTLNCKASENVGTYVSWYQQKPEQSPKLLIYGASNRYTGVPDRFTGSGSATDFTLTISSVQAEDLADYHCGQSYSYPPFTFGSGTKLEIK

SEQ ID NO: 98 - protein, 107 aa, Mus musculus
DIQMTQTTSSLSASLGDRVTISCRASQDISNYLNWYQQKPDGAVKLLIYYTSRLHSGVPSRFSGSGSGTDFSLTISNLEQEDFATYFCQQGNTLPWTFGGGTKLEIK

SEQ ID NO: 99 - protein, 111 aa, Mus musculus; identical to SEQ ID NO: 95

SEQ ID NO: 100 - protein, 107 aa, Mus musculus
DIVMTQSHKFMSTSVGDRVSITCKASQDVGTAVAWYQQKPGQSPKLLIYWASTRHTGVPDRFTGSGSGTDFTLAISNVQSEDLADYFCHQFSSYPLTFGAGTRLELK

SEQ ID NO: 101 - protein, 111 aa, Mus musculus
DIVLTQSPASLAVSLGQRATISCRASENVDNYGISFMHWYQQKPGQPPKFLIYRASNLEYGIPARFSGSGSRTDFTLTINPVETDDVATYYCQQSNKDPLTFGAGTKLELK

SEQ ID NO: 102 - protein, 111 aa, Mus musculus
DIVLTQSPASLAVSLGQRATISCRASKSVSTSGYTYMHWYQQKPGQPPKLLIYLASNLESGVPARFSGSGSGTDFTLNIHPVEEEDAATYYCQHSRELPLTFGAGTKLELK

SEQ ID NO: 103 - protein, 112 aa, Mus musculus
DIVMTQAAFSIPVTLGTSTSISCRSTKSLLHSNGITYLYWYLQKPGQSPQLLIYQMSNLASGVPDRFSSSGSGTDFTLRISRVEAEDVGVYYCAQNLELPWTFGGGTKLEIK

SEQ ID NO: 104 - protein, 112 aa, Mus musculus
DVVMTQTPLSLPVSLGDQASISCRSSQSLVHSNGNTYLHWFLQKPGQSPKLLIYKVSNRFSGVPDRFSGSGSGTDFTLKISRVEAEDLGLYFCSQSTHVPPTFGGGTKLEIK

SEQ ID NO: 105 - protein, 112 aa, Mus musculus
DVVMTQTPLTLSVTIGQPASISCKSTQSLLDSDGKTYLNWFLQRPGQSPKRLIYLVSKLDSGVPDRFTGSGSGTDFTLKISRVEAEDLGLYYCWQGTHFPQTFGGGTKLEIK

SEQ ID NO: 106 - protein, 106 aa, Mus musculus
QIVLTQSPALMSASPGEKVTMTCSASSSVSYMYWYQQKPRSSPKPWIYLTSTLASGVPARFSGSGSGTSYSLTISSMEAEDAATYYCQQWSSNPYTFGGGTKLEIK

SEQ ID NO: 107 - protein, 111 aa, Mus musculus
DIVLTQSPASLAVSLGQRATISCRASESVDNYGISFMHWYQQKPGQPPKLLIYRASNLESGIPARFSGSGSRTDFTLTINPVETDDVATYYCQQSNKDPLTFGAGTKLELK

SEQ ID NO: 108 - protein, 107 aa, Mus musculus
DIVMTQSHKFMSTSVGDRVSITCKASQDVGTAVAWYQQKPGQSPKLLIYWASTRHTGVPDRFTGSGSGTDFTLTISNVQSEDLADYFCQQYSSYPYTFGGGTKLEIK

SEQ ID NO: 109 - protein, 107 aa, Mus musculus
DIVMTQSQKFMSTIVGDRVSITCKASQNVGTAVAWYQQKPGQSPKLLIYSASNRYTGVPDRFTGSGSGTDFTLTISNMQSEDLADYFCQQYSSYTWTFGGGTKLEIK

SEQ ID NO: 110 - protein, 107 aa, Mus musculus
DIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEVK

SEQ ID NO: 111 - protein, 107 aa, Mus musculus
NIVMTQSPKSMSMSVGERVTLSCKAGENVGPYVSWYQQKPEQSPKLLIYGASNRFTGVPDRFTGSGSATDFTLTISSVQAEDLADYHCGQSYSYPFTFGSGTKLEIK

SEQ ID NO: 112 - protein, 112 aa, Mus musculus
DIVMTQAAFSNPVTLGTSASISCRSSKSLLHSNGITYLYWYLQKPGLSPQLLIYHMSNLASGVPDRFSSSGSGTDFTLRISRVEAEDVGVYYCAQNLELPWTFGGGTKLEIK

SEQ ID NO: 113 - protein, 107 aa, Mus musculus
AIQLTQSPSSLSASVGDRVTITCRASENIYSYLAWYQQKPGKAPKLLIYNAKILAEGVPSRFSGSGSGTDFTLTISSLQPEDFATYYCQHHYVSPRTFGQGTKLEIK

SEQ ID NO: 114 - protein, 107 aa, Mus musculus
DIQMTQSPSSLSASVGDRVTITCRASENIYSYLAWYQQKPGKAPKLLIYNAKILAEGVPSRFSGSGSGTDFTLTISSLQPEDFATYYCQHHYVSPRTFGQGTKLEIK

SEQ ID NO: 115 - protein, 10 aa, Homo sapiens
GYSFTSYWIG

SEQ ID NO: 116 - protein, 10 aa, Homo sapiens
GFTFSSYGMH

SEQ ID NO: 117 - protein, 10 aa, Mus musculus
GYTFTNFWMH

SEQ ID NO: 118 - protein, 10 aa, Mus musculus
GYTFTSYTMH

SEQ ID NO: 119 - protein, 10 aa, Mus musculus
GYSFTDYNMN

SEQ ID NO: 120 - protein, 10 aa, Mus musculus
GYTFTDYYID

SEQ ID NO: 121 - protein, 10 aa, Mus musculus
GYTFTDYYMN

SEQ ID NO: 122 - protein, 10 aa, Mus musculus
GYTFTDYYIN

SEQ ID NO: 123 - protein, 12 aa, Mus musculus
GYTFTTYGMSWV

SEQ ID NO: 124 - protein, 7 aa, Mus musculus
GYTINGY

SEQ ID NO: 125 - protein, 10 aa, Mus musculus
GYTFTSYWMH

SEQ ID NO: 126 - protein, 10 aa, Mus musculus
GFSLTNYIIS

SEQ ID NO: 127 - protein, 12 aa, Mus musculus
GYTFTMYGMSWV

SEQ ID NO: 128 - protein, 10 aa, Mus musculus
GSTFTSYVMH

SEQ ID NO: 129 - protein, 10 aa, Mus musculus
GFTFTDYYMH

SEQ ID NO: 130 - protein, 12 aa, Mus musculus
GFSLSTSGMGVS

SEQ ID NO: 131 - protein, 10 aa, Mus musculus
GYTFTDYNMD

SEQ ID NO: 132 - protein, 12 aa, Mus musculus
VYTFTEYPMHWV

SEQ ID NO: 133 - protein, 10 aa, Mus musculus
GFSLTTFGVH

SEQ ID NO: 134 - protein, 10 aa, Mus musculus
GFSLTSYGVH

SEQ ID NO: 135 - protein, 10 aa, Mus musculus
GYTFTDYNMH

SEQ ID NO: 136 - protein, 17 aa, Homo sapiens
IIYPGDSDTRYSPSFQG

SEQ ID NO: 137 - protein, 17 aa, Homo sapiens
FIRYDGSNKYYADSVKG

SEQ ID NO: 138 - protein, 16 aa, Homo sapiens
INPSSDYTKYNQKFKG

SEQ ID NO: 139 - protein, 16 aa, Homo sapiens
INPSSGYTKYNQKFKD

SEQ ID NO: 140 - protein, 16 aa, Mus musculus
INPNYGTTSYNQKFKG

SEQ ID NO: 141 - protein, 16 aa, Mus musculus
IFPGTNSTYYNEKFKG

SEQ ID NO: 142 - protein, 16 aa, Mus musculus
INPYSGGTSYNQKFKG

SEQ ID NO: 143 - protein, 16 aa, Mus musculus
IFPGSGSTYYNEKFKG

SEQ ID NO: 144 - protein, 16 aa, Mus musculus
WINTYSGVPTYADDFK

SEQ ID NO: 145 - protein, 16 aa, Mus musculus
INPNYGTTNYNQKFKG

SEQ ID NO: 146 - protein, 16 aa, Mus musculus
ILPGSGSTNYNEKFKG

SEQ ID NO: 147 - protein, 16 aa, Mus musculus
IDPSESYPNYNQNFKG

SEQ ID NO: 148 - protein, 15 aa, Mus musculus
IWTGGGTNYNSALKS

SEQ ID NO: 149 - protein, 16 aa, Mus musculus
INPNNGGTTYNQKFKG

SEQ ID NO: 150 - protein, 16 aa, Mus musculus
SNPYNDGTKYNEKFKG

SEQ ID NO: 151 - protein, 16 aa, Mus musculus
INPGNGGTDYNEKIKS

SEQ ID NO: 152 - protein, 16 aa, Mus musculus
VYPYNGDTIYNQKFKG

SEQ ID NO: 153 - protein, 16 aa, Mus musculus
ISPSNGGTNYNENFKS

SEQ ID NO: 154 - protein, 15 aa, Mus musculus
IYWDDDKRYNPSLKS

SEQ ID NO: 155 - protein, 16 aa, Mus musculus
INPNNGGTSYNQKFKG

SEQ ID NO: 156 - protein, 16 aa, Mus musculus
INPNNGGTIYNQKFKG

SEQ ID NO: 157 - protein, 16 aa, Mus musculus
WINTYSGEPTYADDFK

SEQ ID NO: 158 - protein, 15 aa, Mus musculus
IWSGGSTDYNAAFIS

SEQ ID NO: 159 - protein, 15 aa, Mus musculus
IWSGGTTDYNAAFIS

SEQ ID NO: 160 - protein, 17 aa, Mus musculus
YIYPYNGGTAYNQKFKN

SEQ ID NO: 161 - protein, 13 aa, Homo sapiens
ARYIQGLGYYFDY

SEQ ID NO: 162 - protein, 13 aa, Homo sapiens
AKPSRGYSRSLDY

SEQ ID NO: 163 - protein, 13 aa, Homo sapiens
ARLQSGWLHAFDI

SEQ ID NO: 164 - protein, 18 aa, Homo sapiens
ARLKWSGLSHYYYYYMDV

SEQ ID NO: 165 - protein, 13 aa, Homo sapiens
ARLPLGLQVGFDY

SEQ ID NO: 166 - protein, 13 aa, Mus musculus
ARVRYSYDLNFDY

SEQ ID NO: 167 - protein, 12 aa, Mus musculus
ARDDYSDFGFAY

SEQ ID NO: 168 - protein, 11 aa, Mus musculus
AREANWDDVDY

SEQ ID NO: 169 - protein, 15 aa, Mus musculus
ARNYYSSSYDGYFDY

SEQ ID NO: 170 - protein, 10 aa, Mus musculus
ARSGLRDFDY

SEQ ID NO: 171 - protein, 12 aa, Mus musculus
ASVSSYGNYFDY

SEQ ID NO: 172 - protein, 13 aa, Mus musculus
AREAKLGRDYFDY

SEQ ID NO: 173 - protein, 13 aa, Mus musculus
ARFPYDFDGYFDV

SEQ ID NO: 174 - protein, 15 aa, Mus musculus
ARNYYGSTYDGYFDY

SEQ ID NO: 175 - protein, 10 aa, Mus musculus
ARGMEGAMDY

SEQ ID NO: 176 - protein, 14 aa, Mus musculus
ARSYYGRSGYAMDY

SEQ ID NO: 177 - protein, 16 aa, Mus musculus
ARNEAVVAIFDWYFDV

SEQ ID NO: 178 - protein, 15 aa, Mus musculus
ARNYYGNNYDGYFDY

SEQ ID NO: 179 - protein, 13 aa, Mus musculus
ARFPYDYDGYFDV

SEQ ID NO: 180 - protein, 15 aa, Mus musculus
ARRYYSSGYDGYFDV

SEQ ID NO: 181 - protein, 11 aa, Mus musculus
ARLNVLYYFDN

SEQ ID NO: 182 - protein, 17 aa, Mus musculus
ARGGGYYGYDGYWYFDV

SEQ ID NO: 183 - protein, 9 aa, Mus musculus
ARGANWGDY

SEQ ID NO: 184 - protein, 7 aa, Mus musculus
ATYYVDY

SEQ ID NO: 185 - protein, 14 aa, Mus musculus
ARRVYGYDPYAMNY

SEQ ID NO: 186 - protein, 15 aa, Mus musculus
APHYYGSSYDWYFDV

SEQ ID NO: 187 - protein, 13 aa, Mus musculus
AREDDSRYWYFDV

SEQ ID NO: 188 - protein, 11 aa, Mus musculus
AREGWLDAMDY

SEQ ID NO: 189 - protein, 9 aa, Mus musculus
APRLGLRAY

SEQ ID NO: 190 - protein, 11 aa, Mus musculus
ARMGGTGYFDV

SEQ ID NO: 191 - protein, 9 aa, Mus musculus
ARGLDVMDY

SEQ ID NO: 192 - protein, 11 aa, Homo sapiens
SGDKLGDKYVS

SEQ ID NO: 193 - protein, 11 aa, Homo sapiens
FGDKLGHKYVS

SEQ ID NO: 194 - protein, 11 aa, Homo sapiens
SGDKLGDKFTS

SEQ ID NO: 195 - protein, 11 aa, Homo sapiens
QGDSLRNYYAS

SEQ ID NO: 196 - protein, 11 aa, Homo sapiens
QGDSLRSYYAS

SEQ ID NO: 197 - protein, 11 aa, Homo sapiens
SGDKLGDKYAS

SEQ ID NO: 198 - protein, 9 aa, Mus musculus
NIYSFLAWY

SEQ ID NO: 199 - protein, 8 aa, Mus musculus
SVSYMYWY

SEQ ID NO: 200 - protein, 14 aa, Mus musculus
SIVHSNGNTYLEWY

SEQ ID NO: 201 - protein, 13 aa, Mus musculus
SVDNYGISFMHWY

SEQ ID NO: 202 - protein, 9 aa, Mus musculus
SISDYLHWY

SEQ ID NO: 203 - protein, 14 aa, Mus musculus
SVSIHASHLLHWYQ

SEQ ID NO: 204 - protein, 9 aa, Mus musculus
DVSTAVAWC

SEQ ID NO: 205 - protein, 9 aa, Mus musculus
DVGTAVAWY

SEQ ID NO: 206 - protein, 9 aa, Mus musculus
DVSTAVAWY

SEQ ID NO: 207 - protein, 9 aa, Mus musculus
NVGTYVSWY

SEQ ID NO: 208 - protein, 9 aa, Mus musculus
DISNYLNWY

SEQ ID NO: 209 - protein, 13 aa, Mus musculus
NVDNYGISFMHWY

SEQ ID NO: 210 - protein, 13 aa, Mus musculus
SVSTSGYTYMHWY

SEQ ID NO: 211 - protein, 14 aa, Mus musculus
SLLHSNGITYLYWY

SEQ ID NO: 212 - protein, 14 aa, Mus musculus
SLVHSNGNTYLHWF

SEQ ID NO: 213 - protein, 14 aa, Mus musculus
SLLDSDGKTYLNWF

SEQ ID NO: 214 - protein, 9 aa, Mus musculus
NVGTAVAWY

SEQ ID NO: 215 - protein, 9 aa, Mus musculus
DINSYLSWF

SEQ ID NO: 216 - protein, 9 aa, Mus musculus
NVGPYVSWY

SEQ ID NO: 217 - protein, 11 aa, Mus musculus
RASENIYSYLA

SEQ ID NO: 218 - protein, 7 aa, Homo sapiens
QDTKRPS

SEQ ID NO: 219 - protein, 7 aa, Homo sapiens
QDSKRPS

SEQ ID NO: 220 - protein, 7 aa, Homo sapiens
QDNKRPS

SEQ ID NO: 221 - protein, 7 aa, Homo sapiens
EDSKRPS

SEQ ID NO: 222 - protein, 7 aa, Homo sapiens
GKNNRPS

SEQ ID NO: 223 - protein, 7 aa, Homo sapiens
QDTKRAS

SEQ ID NO: 224 - protein, 9 aa, Mus musculus
KTLAEGVPS

SEQ ID NO: 225 - protein, 8 aa, Mus musculus
NLASGVPV

SEQ ID NO: 226 - protein, 9 aa, Mus musculus
SNRFSGVPD

SEQ ID NO: 227 - protein, 8 aa, Mus musculus
NLESGIPA

SEQ ID NO: 228 - protein, 8 aa, Mus musculus
QSISGIPS

SEQ ID NO: 229 - protein, 8 aa, Mus musculus
NLESGVPA

SEQ ID NO: 230 - protein, 8 aa, Mus musculus
YRYTGVPD

SEQ ID NO: 231 - protein, 8 aa, Mus musculus
TRHTGVPD

SEQ ID NO: 232 - protein, 8 aa, Mus musculus
NRYTGVPD

SEQ ID NO: 233 - protein, 9 aa, Mus musculus
SRLHSGVPS

SEQ ID NO: 234 - protein, 8 aa, Mus musculus
NLEYGIPA

SEQ ID NO: 235 - protein, 8 aa, Mus musculus
NLASGVPD

SEQ ID NO: 236 - protein, 9 aa, Mus musculus
SKLDSGVPD

SEQ ID NO: 237 - protein, 8 aa, Mus musculus
TLASGVPA

SEQ ID NO: 238 - protein, 9 aa, Mus musculus
NRLVDGVPS

SEQ ID NO: 239 - protein, 8 aa, Mus musculus
NRFTGVPD

SEQ ID NO: 240 - protein, 7 aa, Mus musculus
NAKILAE

SEQ ID NO: 241 - protein, 9 aa, Homo sapiens
QAWDSGTAI

SEQ ID NO: 242 - protein, 9 aa, Homo sapiens
QAWDSSTVV

SEQ ID NO: 243 - protein, 11 aa, Homo sapiens
QVWDSSSDHWV

SEQ ID NO: 244 - protein, 12 aa, Homo sapiens
NSRDSSGNHPVV

SEQ ID NO: 245 - protein, 11 aa, Homo sapiens
NSRDSSGNHLV

SEQ ID NO: 246 - protein, 11 aa, Homo sapiens
NSRDSSGNHVI

SEQ ID NO: 247 - protein, 11 aa, Homo sapiens
QAFDSSAAHFV

SEQ ID NO: 248 - protein, 9 aa, Homo sapiens
QTWDSSTAV

SEQ ID NO: 249 - protein, 9 aa, Mus musculus
QHHYGIPPT

SEQ ID NO: 250 - protein, 9 aa, Mus musculus
QQWSSYPLT

SEQ ID NO: 251 - protein, 9 aa, Mus musculus
FQGSHVPYT

SEQ ID NO: 252 - protein, 9 aa, Mus musculus
QQSNKDPRT

SEQ ID NO: 253 - protein, 9 aa, Mus musculus
QNGHSFPLT

SEQ ID NO: 254 - protein, 9 aa, Mus musculus
QQSIEDPWT

SEQ ID NO: 255 - protein, 8 aa, Mus musculus
QHYSTPYT

SEQ ID NO: 256 - protein, 9 aa, Mus musculus
QQYSSYPLT

SEQ ID NO: 257 - protein, 9 aa, Mus musculus
QQHYSTPPT

SEQ ID NO: 258 - protein, 10 aa, Mus musculus
GQSYSYPPFT

SEQ ID NO: 259 - protein, 9 aa, Mus musculus
QQGNTLPWT

SEQ ID NO: 260 - protein, 9 aa, Mus musculus
HQFSSYPLT

SEQ ID NO: 261 - protein, 9 aa, Mus musculus
QQSNKDPLT

SEQ ID NO: 262 - protein, 9 aa, Mus musculus
QHSRELPLT

SEQ ID NO: 263 - protein, 9 aa, Mus musculus
AQNLELPWT

SEQ ID NO: 264 - protein, 9 aa, Mus musculus
SQSTHVPPT

SEQ ID NO: 265 - protein, 9 aa, Mus musculus
WQGTHFPQT

SEQ ID NO: 266 - protein, 9 aa, Mus musculus
QQWSSNPYT

SEQ ID NO: 267 - protein, 9 aa, Mus musculus
QQYSSYPYT

SEQ ID NO: 268 - protein, 9 aa, Mus musculus
QQYSSYTWT

SEQ ID NO: 269 - protein, 9 aa, Mus musculus
LQYDEFPYT

SEQ ID NO: 270 - protein, 9 aa, Mus musculus
GQSYSYPFT

SEQ ID NO: 271 - protein, 9 aa, Mus musculus
QHHYVSPRT

SEQ ID NO: 272 - protein, 15 aa, unidentified
GGGGSGGGGSGGGGS

SEQ ID NO: 273 - protein, 105 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAY

SEQ ID NO: 274 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 275 - protein, 21 aa, Homo sapiens
IYIWAPLAGTCGVLLLSLVIT

SEQ ID NO: 276 - protein, 28 aa, Homo sapiens
IYIWAPLAGTCGVLLLSLVITLYCNHRN

SEQ ID NO: 277 - protein, 27 aa, Homo sapiens
FWVLVVVGGVLACYSLLVTVAFIIFWV

SEQ ID NO: 278 - protein, 42 aa, Homo sapiens
ALYLLRRDQRLPPDAHKPPGGGSFRTPIQEEQADAHSTLAKI

SEQ ID NO: 279 - protein, 42 aa, Homo sapiens
KRGRKKLLYIFKQPFMRPVQTTQEEDGCSCRFPEEEEGGCEL

SEQ ID NO: 280 - protein, 21 aa, Homo sapiens
GSGEGRGSLLTCGDVEENPGP

SEQ ID NO: 281 - protein, 22 aa, Homo sapiens
GSGATNFSLLKQAGDVEENPGP

SEQ ID NO: 282 - protein, 23 aa, Homo sapiens
GSGQCTNYALLKLAGDVESNPGP

SEQ ID NO: 283 - protein, 25 aa, Homo sapiens
GSGVKQTLNFDLLKLAGDVESNPGP

SEQ ID NO: 284 - protein, 535 aa, Homo sapiens
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDLPGQAKDVVPQAYWLGAPRDHSYEAEFRAVSMKPAFLPSAPGPTMSYYRGQEVLAPGAGWPVAPQYPPKMGPASWFRPMRTLPMEPGPGGSEGRGPEDQGPPLVWTEIAPIRPESSDSGLGEGDSKRRRVSPYPSSGDSSSPAGAPSPFDKEAEGQFYNYFPN

SEQ ID NO: 285 - protein, 260 aa, Homo sapiens
MAQAALEPGEKPYACPECGKSFSTSGSLVRHQRTHTGEKPYKCPECGKSFSRNDALTEHQRTHTGEKPYKCPECGKSFSSKKHLAEHQRTHTGEKPYACPECGKSFSTSGELVRHQRTHTGEKPYKCPECGKSFSRSDKLVRHQRTHTGEKPYKCPECGKSFSRSDHLTEHQRTHTGKKTSGQAGQASPKKKRKVGRADALDDFDLDMLGSDALDDFDLDMLGSDALDDFDLDMLGSDALDDFDLDMLINYPYDVPDYAS

SEQ ID NO: 286 - protein, 211 aa, Homo sapiens
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 287 - protein, 153 aa, Homo sapiens
LKPREWKLVESGGGLVOPGGSLKLSCAASGFDFSRYWMSWVRQAPGKGLEWIGEINLDSSTINYTPSLKDKFIISRDNAKNTLYLOMSKVRSEDTALYYCARRYDAMDYWGQGTSVTVSSAKTTAPSVYPLAPVCGDTTGSSVTLGCLVKASO

SEQ ID NO: 288 - protein, 111 aa, Homo sapiens
ASDIWLTOSPASLAVSLGORATISCRASESVDDYGISFMNWFOOKPGQPPKLLIYAAPNQGSGVPARFSGSGSGTDFSLNIHPMEEDDTAMYFCQQSKDVRWRHQAGDQTG

SEQ ID NO: 289 - protein, 372 aa, Homo sapiens
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 290 - protein, 315 aa, Homo sapiens
MYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

(19) United States
(12) Patent Application Publication
(10) Pub. No.: US 2024/0261405 A1
(43) Pub. Date: Aug. 8, 2024

(54) METHODS AND COMPOSITIONS FOR TREATING AUTOIMMUNE DISEASE

(71) Applicant: Kyverna Therapeutics, Inc., Emeryville, CA (US)

(72) Inventors: John Lee, Alameda, CA (US); Lih-Yun Hsu, San Francisco, CA (US); Jeffrey Greve, Emeryville, CA (US)

(21) Appl. No.: 18/329,959

(22) Filed: Jun. 6, 2023

Related U.S. Application Data
(60) Provisional application No. 63/350,141, filed on Jun. 8, 2022.

(51) Int. Cl. A61K 39/00; A61P 37/06; C07K 14/705; C07K 14/715; C07K 14/725; C07K 16/28; C12N 15/86

(52) U.S. Cl. CPC A61K 39/46433; A61K 39/4611; A61K 39/4631; A61P 37/06; C07K 14/7051; C07K 14/70517; C07K 14/7158; C07K 16/2803; C07K 16/2845; C07K 16/2887; C07K 16/289; C12N 15/86; C07K 2317/622; C07K 2319/02; C07K 2319/03; C07K 2319/30; C12N 2740/15043

(57) ABSTRACT

This document relates to methods and materials for treating a mammal having an autoimmune disease. For example, materials and methods for producing an immuno-activatable cell comprising a first chimeric antigen receptor and a second chimeric antigen receptor are provided. Methods and materials for treating a mammal having an autoimmune disease comprising administering an immuno-activatable cell are also provided.

### CROSS-REFERENCE TO RELATED APPLICATIONS

This application claims priority to U.S. Provisional Patent Application No. 63/350,141, filed on Jun. 8, 2022, the contents of which are incorporated herein by reference in its entirety.

### SEQUENCE LISTING

This application contains a Sequence Listing that has been submitted electronically as an XML file named 47902-0007001_ST26.XML/47902-0007001_ST26/47902-0007TW1_ST26. The XML file, created on Jun. 5, 2023, is 269,901 bytes in size. The material in the XML file is hereby incorporated by reference in its entirety.

### TECHNICAL FIELD

This document relates to methods and materials for treating a mammal having an autoimmune disease. For example, this document provides materials and methods for producing an immuno-activatable cell comprising a first chimeric antigen receptor and a second chimeric antigen receptor. This document also provides methods and materials for treating a mammal having an autoimmune disease comprising administering an immuno-activatable cell.

### BACKGROUND

Autoimmunity is a common disease in the United States, with more than 20 million people suffering from any of 81 known autoimmune diseases. B cells are involved in different aspects of autoimmune diseases, including the secretion of autoantibodies, processing and presentation of autoantigen to T cells, and producing inflammatory cytokines. Age-associated B cells (ABCs) are known to have a role in autoimmune disease (Wang et al., DNA Cell Biol., 35:(10):628-35 (2016)). One contribution to autoimmune disease pathology occurs via ABC production of autoantibodies (Rubtsov et al., Immunol. Res., 55:210-16 (2013)). While autoimmune diseases can be treated with immunosuppressive drugs, there remains an unmet need in the management of ABCs in autoimmune disease.

### SUMMARY

This document provides methods and materials for treating a mammal having an autoimmune disease. For example, this document provides materials and methods for producing an immuno-activatable cell comprising a first chimeric antigen receptor and a second chimeric antigen receptor. This document also provides methods and materials for treating a mammal having an autoimmune disease comprising administering to the mammal an immuno-activatable cell.

In general, one aspect of this document features a method for producing an immuno-activatable cell. The method can comprise (or consist essentially of or consist of) transforming the cell with a first nucleic acid sequence encoding a first chimeric antigen receptor polypeptide. The first chimeric antigen receptor polypeptide can include a first extracellular domain, a first transmembrane domain and a first intracellular domain. The first extracellular domain can include a first antigen binding domain capable of binding to a first antigen on a CD11c⁺Tbet⁺ B cell. The first transmembrane domain can include a first CD8α transmembrane domain. The first intracellular domain can include a cytoplasmic signaling domain. The sequence encoding the first chimeric antigen receptor polypeptide can be operably linked to a first promoter. The method can comprise (or consist essentially of or consist of) transforming the cell with a second nucleic acid sequence encoding a second chimeric antigen receptor polypeptide. The second chimeric antigen receptor polypeptide can include a second extracellular domain, a second transmembrane domain, and a second intracellular domain. The second extracellular domain can include a second antigen binding domain capable of binding to a second antigen on the CD11c⁺Tbet⁺ B cell. The second transmembrane domain can include a second CD8α transmembrane domain. The intracellular domain can include a co-stimulatory domain. The sequence encoding the second chimeric antigen receptor polypeptide can be operably linked to a second promoter. The first antigen binding domain can be an antibody or an antigen binding fragment. The first antigen binding domain can be an antigen binding fragment selected from the group consisting of a Fab, a F(ab′)2 fragment, a scFV, a scab, a dAb, a single domain heavy chain antibody, and a single domain light chain antibody. The first antigen can be a B cell receptor. The B cell receptor can be CD19, CD20, or CD45R.

The first antigen can be CD19. In some embodiments, the first antigen binding domain binds CD19. In some embodiments, the first antigen binding domain comprises an scFv comprising a sequence at least 90% identical to one of SEQ ID NOs: 1-10. In some embodiments, the first antigen binding domain comprises one of the following: (a) a heavy chain variable domain comprising SEQ ID NO: 39 and a light chain variable domain comprising SEQ ID NO: 77; (b) a heavy chain variable domain comprising SEQ ID NO: 40 and a light chain variable domain comprising SEQ ID NO: 78; (c) a heavy chain variable domain comprising SEQ ID NO: 41 and a light chain variable domain comprising SEQ ID NO: 79; (d) a heavy chain variable domain comprising SEQ ID NO: 42 and a light chain variable domain comprising SEQ ID NO: 80; (e) a heavy chain variable domain comprising SEQ ID NO: 43 and a light chain variable domain comprising SEQ ID NO: 81; (f) a heavy chain variable domain comprising SEQ ID NO: 44 and a light chain variable domain comprising SEQ ID NO: 82; (g) a heavy chain variable domain comprising SEQ ID NO: 45 and a light chain variable domain comprising SEQ ID NO: 83; (h) a heavy chain variable domain comprising SEQ ID NO: 46 and a light chain variable domain comprising SEQ ID NO: 84; (i) a heavy chain variable domain comprising SEQ ID NO: 47 and a light chain variable domain comprising SEQ ID NO: 85; or (j) a heavy chain variable domain comprising SEQ ID NO: 48 and a light chain variable domain comprising SEQ ID NO: 86.

The second antigen binding domain can be an antibody or an antigen binding fragment. The second antigen binding domain can be an antigen binding fragment selected from the group consisting of Fab, a F(ab′)2 fragment, a scFV, a scab, a dAb, a single domain heavy chain antibody, and a single domain light chain antibody.

The second antigen can be an antigen present on CD11c⁺Tbet⁺ B cells. The second antigen can be CD11c. In some embodiments, the first antigen binding domain comprises an scFv comprising a sequence at least 90% identical to SEQ ID NO: 37 or SEQ ID NO: 38. In some embodiments, the first antigen binding domain comprises either (a) a heavy chain variable domain comprising SEQ ID NO: 75 and a light chain variable domain comprising SEQ ID NO: 113; or (b) a heavy chain variable domain comprising SEQ ID NO: 76 and a light chain variable domain comprising SEQ ID NO: 114.

The cytoplasmic signaling domain can be a CD3 zeta, CD3 epsilon, CD3 delta, TCR zeta, FcR gamma, FcR beta, CD5, CD22, CD79a, CD79b, or CD66d domain. The co-stimulatory domain can be a CD28, 4-1BB, CD97, CD11a-CD18, CD2, CD27, ICOS, CD154, CD5, or OX40 signaling domain. The first and second CD8α transmembrane domains can include a CD8α hinge domain and a CD8α stalk domain. The cell can be further transformed by transforming the cell with a third nucleic acid encoding a first chemokine receptor polypeptide. The cell can be further transformed by transforming the cell with a fourth nucleic acid encoding a second chemokine receptor polypeptide. The cell can be further transformed by transforming the cell with a third nucleic acid encoding a first chemokine receptor polypeptide and a fourth nucleic acid encoding a second chemokine receptor polypeptide. The first or second chemokine receptor polypeptides can be a receptor present on a lymphoid cell. The chemokine receptor can be a CXCR5 or CCR7. The third nucleic acid can encode a CXCR5 polypeptide and the fourth nucleic acid can encode a CCR7 polypeptide. The immuno-activatable cell can be an immune cell selected from the group consisting of a T cell, a B cell, a monocyte, a natural killer cell, a dendritic cell, a macrophage, a regulatory T cell, a helper T cell, and a cytotoxic T cell.

In another aspect, this document features an immuno-activatable cell produced by the methods described herein.

In another aspect, this document features a method for treating a mammal having an autoimmune disease. The method can comprise (or consist essentially of or consist of) administering to the mammal an effective amount of the immuno-activatable cell as produced by the methods and materials as described herein. For example, the immuno-activatable cell can express a first chimeric antigen receptor polypeptide having a first antigen binding domain that binds a first antigen on a CD11c⁺Tbet⁺ B cell with low affinity, where the binding activates the immuno-activatable cell. The immuno-activatable cell also can express a second chimeric antigen receptor polypeptide having a second antigen binding domain that binds a second antigen on a CD11c⁺Tbet⁺ B cell and stimulates the immuno-activatable cell, thereby treating or preventing an autoimmune disorder in the mammal. The mammal can be human. The autoimmune disease can result from production of autoantibodies by Age-associated B cells. The autoimmune disease can be lupus, rheumatoid arthritis, multiple sclerosis, insulin dependent diabetes mellitis, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, Crohn's disease, Celiac disease, or polyarteritis nodosa. The method can reduce the number of age-associated B-cells.

In another aspect, this document features a method for producing a T cell targeting Age-associated B cells. The method can comprise (or consist essentially of or consist of) transforming the T cell with a first nucleic acid sequence encoding a first chimeric antigen receptor polypeptide. The first chimeric antigen receptor can include an extracellular domain, a transmembrane domain and an intracellular domain. The extracellular domain can include a first antigen binding domain capable of binding to a CD19 antigen on the surface of a CD11c⁺Tbet⁺ B cell. The first transmembrane domain can include a first CD8α transmembrane domain. The first intracellular domain comprises CD3zeta domain. The sequence encoding the first chimeric antigen receptor polypeptide can be operably linked to a first promoter. The method can comprise (or consist essentially of or consist of) transforming the cell with a second nucleic acid sequence encoding a second chimeric antigen receptor polypeptide. The second chimeric antigen receptor can include a second extracellular domain, a second transmembrane domain and a second intracellular domain. The second extracellular domain can include a second antigen binding domain capable of binding to a CD11c antigen on the surface of a CD11c⁺Tbet⁺ B cell. The second transmembrane domain can include a second CD8α transmembrane domain. The second intracellular domain comprises a CD28 signaling domain. The sequence encoding the second chimeric antigen receptor polypeptide can be operably linked to a second promoter. The cell can be further transformed by transforming the cell with a third nucleic acid encoding a first chemokine receptor polypeptide. The cell can be further transformed by transforming the cell with a fourth nucleic acid encoding a second chemokine receptor polypeptide. The cell can be further transformed by transforming the cell with a third nucleic acid encoding a first chemokine receptor polypeptide and a fourth nucleic acid encoding a second chemokine receptor polypeptide. The first or second chemokine polypeptide can be a receptor present on a lymphoid cell. The chemokine receptor polypeptide can be a CXCR5 or CCR7. The third nucleic acid can encode a CXCR5 polypeptide and the fourth nucleic acid can encode a CCR7 polypeptide.

In another aspect, this document features a method for treating a mammal having an autoimmune disease. The method can comprise (or consist essentially of or consist of) administering to the mammal an effective amount of the immuno-activatable cell as produced by methods and materials described herein, where the T cell expresses a first chimeric antigen receptor polypeptide having a first antigen binding domain that binds a CD19 antigen on a CD11c⁺Tbet⁺ B cell with low affinity, where the binding activates the T cell, and where the T cell expresses a second chimeric antigen receptor polypeptide having a second antigen binding domain that binds a CD11c antigen on a CD11c⁺Tbet⁺ B cell and stimulates the T cell, thereby treating or preventing an autoimmune disease in the mammal. The mammal can be a human. The autoimmune disease can result from production of autoantibodies by age-associated B cells. The autoimmune disease can be lupus, rheumatoid arthritis, multiple sclerosis, insulin dependent diabetes mellitis, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, Crohn's disease, Celiac disease, or polyarteritis nodosa. The method can reduce the number of age-associated B cells in the mammal.

In another aspect, this document features a composition containing a chimeric antigen receptor polypeptide that includes an extracellular domain, a transmembrane domain, and an intracellular domain. The extracellular domain can include an antigen binding domain capable of binding to an antigen on a CD11c⁺Tbet⁺ B cell. The transmembrane domain can include a CD8α transmembrane domain. The intracellular domain can include a cytoplasmic signaling domain. The antigen binding domain can be an antibody or antigen binding fragment. The antigen binding domain can be an antigen binding fragment selected from the group consisting of a Fab, a F(ab′)2 fragment, a scFV, a scab, a dAb, a single domain heavy chain antibody, and a single domain light chain antibody. The antigen can be a B cell receptor. The B cell receptor can be CD19, CD20, or CD45R. The antigen can be CD19. The antigen can be an antigen present on CD11c⁺Tbet⁺ B cells. The antigen can be CD11c. The cytoplasmic signaling domain can be a CD3zeta, CD3epsilon, CD3delta, TCRzeta, FcR gamma, FcR beta, CD5, CD22, CD79a, CD79b or CD66d domain.

In some embodiments where the chimeric antigen receptor polypeptide includes a CD3 zeta cytoplasmic signaling domain, the CD3 zeta cytoplasmic signaling domain has an amino acid sequence that is at least 80% (e.g., at least 85%, 90%, 95%, 99% and 100%) identical to SEQ ID NO: 274 (NCBI Reference No: NP_932170) (SEQ ID NO: 274) or a fragment thereof that has activating or stimulatory activity.

In another aspect, this document features a composition containing a second chimeric antigen receptor polypeptide containing an extracellular domain, a transmembrane domain and an intracellular domain. The extracellular domain can include an antigen binding domain capable of binding to an antigen on a CD11c⁺Tbet⁺ B cell. The transmembrane domain can include a CD8α transmembrane domain. The intracellular domain can include a co-stimulatory domain. The antigen binding domain can be an antibody or antigen binding fragment. The antigen binding domain can be an antigen binding fragment selected from the group consisting of a Fab, a F(ab′)2 fragment, a scFV, a scab, a dAb, a single domain heavy chain antibody, and a single domain light chain antibody. The antigen can be a B cell receptor. The B cell receptor can be CD19, CD20, or CD45R. The antigen can be CD19. In some embodiments, the antigen binding domain comprises an scFv comprising a sequence at least 90% identical to one of SEQ ID NOs: 1-10. In some embodiments, the antigen binding domain comprises one of the following: (a) a heavy chain variable domain comprising SEQ ID NO: 39 and a light chain variable domain comprising SEQ ID NO: 77; (b) a heavy chain variable domain comprising SEQ ID NO: 40 and a light chain variable domain comprising SEQ ID NO: 78; (c) a heavy chain variable domain comprising SEQ ID NO: 41 and a light chain variable domain comprising SEQ ID NO: 79; (d) a heavy chain variable domain comprising SEQ ID NO: 42 and a light chain variable domain comprising SEQ ID NO: 80; (e) a heavy chain variable domain comprising SEQ ID NO: 43 and a light chain variable domain comprising SEQ ID NO: 81; (f) a heavy chain variable domain comprising SEQ ID NO: 44 and a light chain variable domain comprising SEQ ID NO: 82; (g) a heavy chain variable domain comprising SEQ ID NO: 45 and a light chain variable domain comprising SEQ ID NO: 83; (h) a heavy chain variable domain comprising SEQ ID NO: 46 and a light chain variable domain comprising SEQ ID NO: 84; (i) a heavy chain variable domain comprising SEQ ID NO: 47 and a light chain variable domain comprising SEQ ID NO: 85; or (j) a heavy chain variable domain comprising SEQ ID NO: 48 and a light chain variable domain comprising SEQ ID NO: 86.

The antigen can be an antigen present on CD11c⁺Tbet⁺ B cells. The antigen can be CD11c. In some embodiments, the scFv fragment comprises a sequence at least 90% identical to SEQ ID NO: 37 or SEQ ID NO: 38. In some embodiments, the scFv fragment comprises either: (a) a heavy chain variable domain comprising SEQ ID NO: 75 and a light chain variable domain comprising SEQ ID NO: 113; or (b) a heavy chain variable domain comprising SEQ ID NO: 76 and a light chain variable domain comprising SEQ ID NO: 114.

The co-stimulatory domain can be a CD28, 4-1BB, CD97, CD11a-CD18, CD2, CD27, ICOS, CD154, CD5, or OX40.

Also provided herein are nucleic acids encoding any of the chimeric antigen receptor polypeptides described herein. Some embodiments include a vector containing a nucleic acid as described herein. In some embodiments, the vector can be an expression vector. Also provided herein are cells transformed with the nucleic acids and/or vectors described herein. The cells can be selected from the group consisting of a T cell, a B cell, a monocyte, a natural killer cell, a dendritic cell, a macrophage, a regulatory T cell, a helper T cell, and a cytotoxic T cell. Also provided herein is a pharmaceutical composition including one or more chimeric antigen receptor polypeptides as described herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is an exemplary diagram showing combinatorial recognition of CD11c⁺T-bet⁺ B cells with an immuno-activatable cell expressing a chimeric antigen receptor polypeptide targeting CD19 and a chimeric antigen receptor targeting CD11c as described herein. The CD11c⁺T-bet⁺ B cells express CD19 and CD11c on the surface.

FIG. 2A shows an exemplary construct that encodes a polypeptide comprising a EF1α signal sequence, an anti-CD19 scFv, a CD8 stalk, a CD8 transmembrane domain, a CD3ζ intracellular domain, a P2A protease cleavage site, and CDCR5/CCR7, that can be used to generate immune activatable cells.

FIG. 2B shows an exemplary construct that encodes a polypeptide comprising a EF1α signal sequence, an anti-CD19 scFv, a CD8 stalk, a CD8 transmembrane domain, and a CD3ζ intracellular domain, that can be used to generate immune activatable cells.

FIG. 2C shows an exemplary construct that encodes a polypeptide comprising a EF1α signal sequence, an anti-CD11c scFv, a CD8 stalk, a CD8 transmembrane domain, a 41BB/CD28 intracellular domain, a P2A protease cleavage site, and CXCR5/CCR7, that can be used to generate immune activatable cells.

FIG. 2D shows an exemplary construct that encodes a polypeptide comprising a EF1α signal sequence, an anti-CD11c scFv, a CD8 stalk, a CD8 transmembrane domain, and a 41BB/CD28 intracellular domain, that can be used to generate immune activatable cells.

FIG. 2E shows an exemplary construct that encodes a polypeptide comprising a EF1α signal sequence, an anti-CD11c scFv, a CD8 stalk, a CD8 transmembrane domain, a 41BB or CD28 intracellular domain, a P2A protease cleavage site, an anti-CD19 scFv, a CD8 stalk, a CD8 transmembrane domain, a CD3ζ intracellular domain, a P2A protease cleavage site, and CXCR5/CCR7, that can be used to generate immune activatable cells.

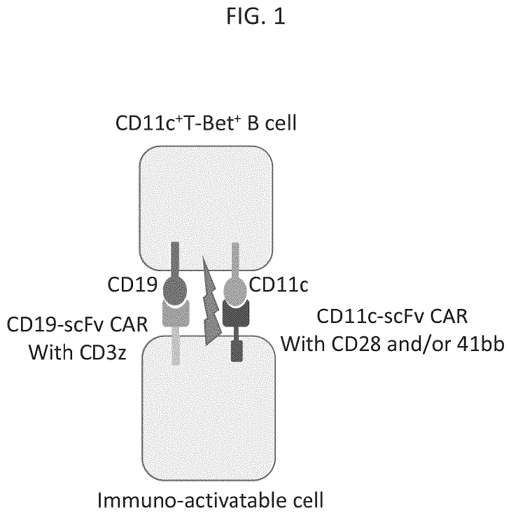

### DETAILED DESCRIPTION

This document relates to methods and materials for treating a mammal having an autoimmune disease. For example, this document provides materials and methods for producing an immuno-activatable cell that expresses or contains a first chimeric antigen receptor and a second chimeric antigen receptor. This document also provides methods and materials for treating a mammal identified as having an autoimmune disease, where the methods include administering an immuno-activatable cell as described herein. In some cases, the methods and compositions provided herein include treating a disease in a patient, including an autoimmune disease or cancer, by administering to the patient a cell transformed with any of the nucleic acids, vectors, or polypeptides described herein.

In some embodiments, this document provides methods and materials for treating a mammal having an autoimmune disease resulting from the production of autoantibodies by age-associated B cells (ABCs). In some cases, a method of treating an autoimmune disease, as described herein, can include by administering to the mammal an immune cell (e.g., a T cell) genetically altered to encode and express two or more chimeric antigen receptors (e.g., an immuno-activatable cell). In some cases, an immune cell (e.g., a T cell) with two or more CARs expressed on its surface is capable of inducing and sustaining an immune response. In some cases, an immune cell can be genetically altered to contain two CARs designed to bind to antigens on the surface of ABCs. In some cases, binding of CARs to antigens on the surface of ABCs can result in an immune response against the ABCs, and possibly elimination of the ABCs. In some cases, only when both antigens are present on the surface of an ABC will CAR signaling occur (e.g., activated and sustained immune response against the ABC). In some cases, only when both antigens are present on the surface of an ABC and the cell contains the appropriate combination of two or more CARs will CAR signaling occur (e.g., activated and sustained immune response against the ABC). For example, an activated and sustained immune response can be achieved when a first CAR containing a cytoplasmic signaling domain activates CAR signaling upon binding of the CAR to an antigen, and a second CAR containing a co-stimulatory domain that stimulates CAR signaling upon binding of the CAR to a second antigen.

To ensure that CAR activation does not occur due to only one of the antibodies or antigen binding fragments binding its target antigen, the affinity of the antibodies can be adjusted so that CAR activation, and thus ABC killing, occurs only when there is a combined effect of both antibodies. In general, CAR activation can depend on the function of an antigen binding fragment, antibody affinity, antigen density of either the antigens for either CARs, and/or the antigen selectively of the CARs. In some cases, altering the binding affinity of a CAR can be achieved by empirically determining the antigen density and antigen selectivity. In some cases, one CAR can be designed to have lower affinity. In some cases, one CAR can be designed to have higher affinity.

In some cases, empirical testing can be done to determine which CAR (e.g., which antigen binding fragment) contains should contain which intracellular domain. In some cases, the scFv CD19 can have a binding affinity in the micromolar range, while the scFv CD11c will preferably be in the nanomolar range. In some cases, the actual affinities of both scFvs can be determined by determining their corresponding antigen densities present on an ABC cell. For example, empirical determination of antigen density on an ABC can allow determination of actual binding affinities favorable for CAR activation, and thus the elimination or reduction of ABC cells (see, e.g., Kloss et al., Nat. Biotechnol., 31:71-75 (2013)).

As used herein, the term “ABC cells” also termed double negative B cells, atypical memory B-cells, or tissue-like memory B-Cells that exhibit the cell surface receptor CD11c and the T-Box transcription factor (T-bet). Unlike other B cells, ABCs express CD11c, a receptor also expressed in myeloid cells. T-bet is a transcription factor known for its role as a master regulator of commitment of T cells to the T helper 1 cell lineage. ABC cells are also referred to as CD11c⁺T-bet⁺ B cells (see, Karnell et al., Cellular Immunol., 321: 40-50 (2017)).

As used herein, the term “chimeric antigen receptor” or “CAR” as used herein refers to comprising chimeric receptor comprising an extracellular domain, a transmembrane domain, and an intracellular domain. In some cases, the extracellular domain can comprise an antigen binding domain as described herein. In some cases, the transmembrane domain can comprise a transmembrane domain derived from a natural polypeptide obtained from a membrane-binding or transmembrane protein. For example, a transmembrane domain can include, without limitation, a transmembrane domain from a T cell receptor alpha or beta chain, a CD3 zeta chain, a CD28 polypeptide, or a CD8 polypeptide. In some cases, the intracellular domain can comprise a cytoplasmic signaling domain as described herein. In some cases, the intracellular domain can comprise a co-stimulatory domain as described herein.

In some embodiments, the scFv comprises a light chain variable domain comprising a sequence that is at least 90% identical (e.g., at least 91%, 92%, 93%, 94%, 95%, 96%, 97%, 98%, 99%, or 100% identical) to one of SEQ ID NOs: 77-114. In some embodiments, the scFv comprises a heavy chain variable domain comprising a sequence that is at least 90% identical (e.g., at least 91%, 92%, 93%, 94%, 95%, 96%, 97%, 98%, 99%, or 100% identical) to one of SEQ ID NOs: 39-76.

As used herein, the term “activation” refers to induction of a signal on an immune cell (e.g., a B cell or T cell) that can result in initiation of the immune response (e.g., T cell activation). In some cases, upon binding of an antigen (e.g., CD19 or CD11c) to a T cell receptor (TCR) or an exogenous chimeric antigen receptor (CAR), the immune cell can undergo changes in protein expression that result in the activation of the immune response. In some cases, a TCR or CAR includes a cytoplasmic signaling sequence that can drive T cell activation. For example, upon binding of the antigen, a chimeric antigen receptor comprising an intracellular domain that includes a cytoplasmic signaling sequence (e.g., an immunoreceptor tyrosine-based inhibition motifs (ITAM)) that can be phosphorylated. A phosphorylated ITAM results in the induction of a T cell activation pathway that ultimately results in a T cell immune response. Examples of ITAMs include, without limitation, CD3 gamma, CD3 delta, CD3 epsilon, TCR zeta, FcR gamma, FcR beta, CD5, CD22, CD79a, and CD66d.

The term “affinity” as used herein, refers to the binding of mutant porcine IL-2 to the human IL-2 receptor, trimeric or dimeric forms. Affinity can be measured using any suitable method. See, e.g., Shanafelt et al., 2000 Nature Biotechnol 18: 1197-1202.

As used herein, the term “stimulation” refers to stage of TCR or CAR signaling where a co-stimulatory signal can be used to achieve a robust and sustained TCR or CAR signaling response. As described herein, a co-stimulatory domain can be referred to as a signaling domain. In some cases, a signaling domain (e.g., co-stimulatory domain) can be a CD27, CD28, OX40, CD30, CD40, B7-H3, NKG2C, LIGHT, CD7, CD2, 4-1BB, PD-1, or LFA-1.

In some embodiments where the chimeric antigen receptor polypeptide includes a CD28 co-stimulatory domain, the CD28 co-stimulatory domain is at least 80% (e.g., at least 85%, 90%, 95%, 99% and 100%) identical to SEQ ID NO: 273.

In some embodiments where the chimeric antigen receptor polypeptide includes a OX40 co-stimulatory domain, the OX40 co-stimulatory domain is at least 80% (e.g., at least 85%, 90%, 95%, 99% and 100%) identical to SEQ ID NO: 278.

In some embodiments where the chimeric antigen receptor polypeptide includes a 4-1BB co-stimulatory domain, the 4-1BB co-stimulatory domain is at least 80% (e.g., at least 85%, 90%, 95%, 99% and 100%) identical to SEQ ID NO: 279.

In some cases, the transformed cell is genetically modified with a nucleic acid comprising a nucleotide sequence encoding a chimeric antigen receptor (CAR), and wherein the intracellular domain of the mutated chimeric NOTCH polypeptide is a transcriptional activator or repressor. In some cases, the nucleic acid sequence encoding the CAR is operably linked to a transcriptional control element that is activated by the intracellular domain of the mutated chimeric NOTCH polypeptide. In some embodiments, the cell to be transformed already stably expresses a CAR. Any appropriate mammal having an autoimmune disease can be treated by administering to the mammal an immuno-activatable cell comprising two or more chimeric antigen receptors (CARs). For example, humans or other primates such as monkeys having an autoimmune disease can be treated by administering to the mammal an immuno-activatable cell comprising two or more CARs as described herein. In some cases, dogs cats, horses, cows, pigs, sheep, mice, or rats having an autoimmune disease can be treated by administering an immuno-activatable cell as described herein.

In some embodiments, the intracellular domain comprises a transcriptional activation domain. In some embodiments, the transcriptional activation domains is selected from the group comprising a VP 16 activation domain, a VP64 activation domain, a p65 activation domain, a MyoDl activation domain, a Tbx21 activation domain a HSF1 activation domain, a RTA activation domain, a SET7/9 activation domain, a Gal4 DNA binding domain (DBD)-VP64 domain, a tTA-VP64: tetR-VP64 domain, a VP64-p65-Rta (VPR) activation domain, a mini VPR activation domain, a yeast GAL4 activation domain, a yeast HAP1 activation domain, a histone acetyltransferase, or any combination thereof.

In some embodiments where the chimeric antigen receptor polypeptide includes a Tbx21 transcriptional activation domain, the Tbx21 transcriptional activation domain is at least 80% (e.g., at least 85%, 90%, 95%, 99% and 100%) identical to SEQ ID NO: 284.

In some embodiments where the chimeric antigen receptor polypeptide includes a E2S-VP64 transcriptional activation domain, the E2S-VP64 transcriptional activation domain is at least 80% (e.g., at least 85%, 90%, 95%, 99% and 100%) identical to SEQ ID NO: 285.

In some embodiments where the chimeric antigen receptor polypeptide includes a GAL4-VP64 transcriptional activation domain, the GAL4-VP64 transcriptional activation domain is at least 80% (e.g., at least 85%, 90%, 95%, 99% and 100%) identical to SEQ ID NO: 286.

As used herein, the term “antibody,” “antigen binding domain,” or antigen binding fragment” refers to an intact immunoglobulin or to an antigen binding portion thereof. Antigen binding portions are well known in the art and may be produced by recombinant DNA techniques or by enzymatic or chemical cleavage of intact antibodies. Examples of antigen binding portions include Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide. As used herein, the term “scFv” antibody fragments comprise the V_{H} and V_{L} domains of antibody, wherein these domains are present in a single polypeptide chain. Included in the definition are single domain antibody, including camelids. In some cases, the antibody is human or humanized.

In some embodiments, any of the “antigen binding domain,” “antibody,” or “ligand binding domain” described herein can bind specifically to a target selected from the group of: CD16a, CD28, CD3 (e.g., one or more of CD3α, CD3β, CD3δ, CD3ε, and CD3γ), CD33, CD20, CD19, CD22, CD123, IL-1R, IL-1, VEGF, IL-6R, IL-4, IL-10, PDL-1, TIGIT, PD-1, TIM3, CTLA4, MICA, MICB, IL-6, IL-8, TNFα, CD26a, CD36, ULBP2, CD30, CD200, IGF-1R, MUC4AC, MUC5AC, Trop-2, CMET, EGFR, HER1, HER2, HER3, PSMA, CEA, B7H3, EPCAM, BCMA, P-cadherin, CEACAM5, a UL16-binding protein (e.g., ULBP1, ULBP2, ULBP3, ULBP4, ULBP5, and ULBP6), HLA-DR, DLL4, TYRO3, AXL, MER, CD122, CD155, PDGFDD, a ligand of TGF-β receptor II (TGF-βRII), a ligand of TGF-βRIII, a ligand of DNAMI, a ligand of NKp46, a ligand of NKp44, a ligand of NKG2D, a ligand of NK30, a ligand for a scMHCI, a ligand for a scMHCII, a ligand for a scTCR, a receptor for IL-1, a receptor for IL-2, a receptor for IL-3, a receptor for IL-7, a receptor for IL-8, a receptor for IL-10, a receptor for IL-12, a receptor for IL-15, a receptor for IL-17, a receptor for IL-18, a receptor for IL-21, a receptor for PDGF-D, a receptor for stem cell factor (SCF), a receptor for stem cell-like tyrosine kinase 3 ligand (FLT3L), a receptor for MICA, a receptor for MICB, a receptor for a ULP16-binding protein, a receptor for CD155, a receptor for CD122, and a receptor for CD28.

In some embodiments of these chimeric antigen receptor polypeptides described herein, the first antigen binding domain and the second antigen binding domain bind specifically to the same antigen. In some embodiments of these chimeric antigen receptor polypeptides, the first antigen binding domain and the second antigen binding domain bind specifically to the same epitope. In some embodiments of these chimeric antigen receptor polypeptides, the first antigen binding domain and the second antigen binding domain include the same amino acid sequence. In some embodiments of any of these chimeric antigen receptor polypeptides described herein, the first antigen binding domain and the second antigen binding domain bind specifically to different antigens.

The antigen-binding domains present in any of the chimeric antigen receptor polypeptides described herein are each independently selected from the group consisting of: a VHH domain, a VNAR domain, and a scFv.

### Methods of Producing Immuno-Activatable T Cells

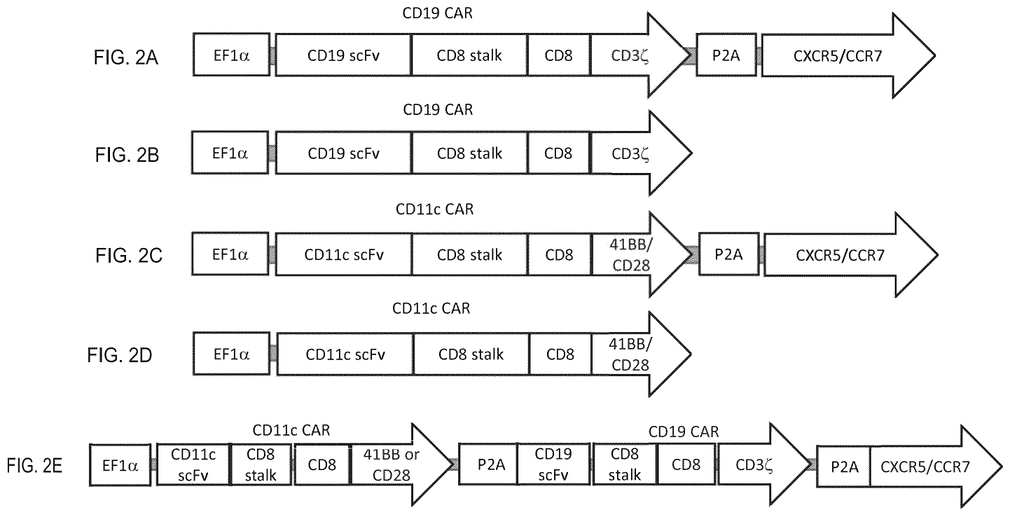

As described herein, any appropriate method of producing immune cells (e.g., T cells) comprising chimeric antigen receptors (CAR) and chemokine receptor polypeptides can be used to generate the immuno-activatable cells as described herein. In some cases, a combination of nucleic acid sequences encoding the domains listed in FIG. 2A or 2B including the operably linked promoters can be transformed into an immune cell (e.g., a T cell) along with a nucleic acid sequence encoding the domains listed in FIG. 2C or 2D including the operably linked promoters. For example, an immuno-activatable cell can be made by co-transducing the exemplary constructs of FIGS. 2A and 2C into an immune cell (e.g., a T cell) using a lentivirus. In some cases, an immuno-activatable cell can be made by transducing the nucleic acid sequence encoding the domains listed in FIG. 2E into an immune cell (e.g., a T cell) using a lentivirus.

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection.

In some embodiments, the transformed cell can be an immune cell, a neuron, an epithelial cell, an endothelial cell, or a stem cell. In some embodiments, the transformed cell is an immune cell selected from the group consisting of a T cell, a B cell, a natural killer (NK) cell, a dendritic cell, a macrophage, a regulatory T cell, a helper T cell and a cytotoxic T cell. In some examples, the immune cell is a NK cell, and the detection of a memory NK cell can include, e.g., the detection of the level of one or more of IL-12, IL-18, IL-33, STAT4, Zbtb32, DNAM-1, BIM, Noxa, SOCS1, BNIP3, BNIP3L, interferon-γ, CXCL16, CXCR6, NKG2D, TRAIL, CD49, Ly49D, CD49b, and Ly79H. A description of NK memory cells and methods of detecting the same is described in O'Sullivan et al., Immunity 43:634-645, 2015. In some examples, the immune cell is a T cell, and the detection of memory T cells can include, e.g., the detection of the level of expression of one or more of CD45RO, CCR7, L-selectin (CD62L), CD44, CD45RA, integrin αeβ7, CD43, CD27, CD28, IL-7Rα, CD95, IL-2Rβ, CXCR3, and LFA-1. In some examples, the immune cell is a B cell and the detection of memory B cells can include, e.g., the detection of the level of expression of CD27. Other types and markers of memory or memory-like immune cells are known in the art.

### Chemokine Receptor Polypeptides

As described herein, this document features a method for generating an immuno-activatable cell comprising two or more CARs and one or more chemokine receptor polypeptides. In some cases, an immuno-activatable cell can be generated by transforming an immune cell (e.g., a T cell) with nucleic acid sequences encoding two or more CARs as described herein and nucleic acid sequences encoding one or more chemokine receptor polypeptides. In some cases, chemokine receptor polypeptides can be specifically designed so that when expressed on the surface the polypeptides can target the transformed immune cell to lymphoid tissue. For example, an immuno-activatable cell can be generated by transforming an immune cell (e.g., a T cell) with nucleic acid sequences encoding two or more CARs and a nucleic acid sequence encoding a CXCR5 chemokine receptor polypeptide. A CXCR5 chemokine receptor polypeptide can direct the transformed immune cell to lymphoid tissue follicles (Hughes et al., FEBS J., 285(16):2944-71 (2018)). In another example, an immuno-activatable cell can be generated by transforming an immune cell (e.g., a T cell) with nucleic acid sequences encoding two or more CARs and a nucleic acid sequence encoding a CCR7 chemokine receptor polypeptide. A CCR7 chemokine receptor polypeptide can direct the transformed immune cell (e.g., T cell) to lymphatic tissue and/or the thymus (Hughes et al., FEBS J., 285(16):2944-71 (2018)). Other examples of chemokine receptor polypeptides that can be used to direct a transformed immune cell to a specific location in the body (e.g., lymphoid tissue follicles or thymus tissue) include, without limitation, CCR2, CCR3, CCR4, CCR5, CCR8, CCR9, CXCR1, CXCR2, CXCR, 3 CXCR4, CXCR6, and CRTH2.

### Nucleic Acids/Vectors

Also provided herein are nucleic acids sequences that encode any of the chimeric antigen receptor polypeptides described herein. Also provided herein are vectors that include any of the nucleic acids encoding any of the chimeric antigen receptor polypeptides described herein.

Any of the vectors described herein can be an expression vector. For example, an expression vector can include a promoter sequence operably linked to the sequence encoding the chimeric antigen receptor polypeptides. Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. A vector can, e.g., include sufficient cis-acting elements for expression; other elements for expression can be supplied by the host mammalian cell or in an in vitro expression system. Skilled practitioners will be capable of selecting suitable vectors and mammalian cells for making any of the immuno-activatable cells as described herein. Any appropriate promoter (e.g., EF1 alpha) can be operably linked to any of the nucleic acid sequences described herein. As used herein, the term “operably linked” is well known in the art and refers to genetic components that are combined such that they carry out their normal functions. For example, a gene is operably linked to a promoter when its transcription is under the control of the promoter. In another example, a nucleic acid sequence can be operable linked to another nucleic acid sequence by a self-cleaving 2A polypeptide. In such cases, the self-cleaving 2A polypeptide allows the second nucleic acid to be under the control of the promoter operably linked to the first nucleic acid sequence and allows the second nucleic acid to be in frame with the first nucleic acid.

In some embodiments the T2A cleavage sequence (GSGEGRGSLLTCGDVEENPGP (SEQ ID NO: 280)), a P2A cleavage sequence (GSGATNFSLLKQAGDVEENPGP (SEQ ID NO: 281)), a E2A cleavage sequence (GSGQCTNYALLKLAGDVESNPGP (SEQ ID NO: 282)) or a F2A cleavage sequence GSGVKQTLNFDLLKLAGDVESNPGP (SEQ ID NO: 283)).

In some cases, an exemplary nucleic acid sequence used to make an immuno-activatable cell as described herein can include a promoter operably linked to nucleic acid sequences encoding a CAR comprising an antigen binding domain capable of binding to antigen on a CD11c⁺Tbet⁺ B cell, a CD8α transmembrane domain comprising a CD8α stalk and a CD8α hinge region, and a cytoplasmic signaling domain. In some cases, an exemplary nucleic acid sequence used make an immuno-activatable cell as described herein can include a promoter operably linked to nucleic acid sequences encoding a CAR comprising an antigen binding domain capable of binding to antigen on a CD11c⁺Tbet⁺ B cell, a CD8α transmembrane domain comprising a CD8α stalk and a CD8α hinge region, and a co-stimulatory domain. In some cases, an exemplary nucleic acid sequence used to make an immuno-activatable cell as described herein can include a promoter operably linked to nucleic acid sequences encoding a CAR comprising an antigen binding domain capable of binding to antigen on a CD11c⁺Tbet⁺ B cell, a CD8α transmembrane domain comprising a CD8α stalk and a CD8α hinge region, a cytoplasmic signaling domain, and a chemokine receptor polypeptide operably linked to the CAR (e.g., in frame) with a self-cleaving 2A sequence (e.g., a P2A, a T2A, a E2A or a F2A) (FIG. 2A). In some cases, an additional chemokine receptor polypeptide can be operably linked to the 3′ end of the first chemokine receptor polypeptide using a second self-cleaving 2A polypeptide sequence (e.g., a P2A, a T2A, a E2A or a F2A). For example, a nucleic acid sequence used to make an immuno-activatable cell can include sequences encoding a CAR comprising an antigen binding domain capable of binding to antigen on a CD11c⁺Tbet⁺ B cell, a CD8α transmembrane domain comprising a CD8α stalk and a CD8α hinge region, a cytoplasmic signaling domain, a first chemokine receptor polypeptide operably linked to the CAR (e.g., in frame) with a self-cleaving 2A sequence (e.g., a P2A, a T2A, a E2A or a F2A), and a second chemokine receptor polypeptide operably linked to the first nucleic acid sequence with a self-cleaving 2A sequence (e.g., a P2A, a T2A, a E2A or a F2A). In some cases, the chemokine receptor polypeptide can be a CXCR5. In some cases, the chemokine receptor polypeptide can be a CCR7.

In some cases, the nucleic acid sequences encoding a CAR as used herein can include a sequence from 5′ to 3′ a promoter operably linked to nucleic acid sequences encoding a scFv antigen binding domain capable of binding to CD19, a CD8α transmembrane domain comprising a CD8α stalk and a CD8α hinge region, and a CD3zeta cytoplasmic signaling domain (FIG. 2B). In some cases, the nucleic acid sequences encoding a CAR can also include a chemokine receptor polypeptide. For example, a nucleic acid sequence encoding a CAR can include a promoter operably linked to nucleic acid sequences encoding a scFv antigen binding domain capable of binding to CD19, a CD8α transmembrane domain comprising a CD8α stalk and a CD8α hinge region, a CD3zeta cytoplasmic signaling domain, and a chemokine receptor polypeptide operably linked to the CAR (e.g., in frame) with a self-cleaving 2A sequence (e.g., a P2A, a T2A, a E2A or a F2A). In some cases, the chemokine receptor polypeptide can be a CXCR5. In some cases, the chemokine receptor polypeptide can be a CCR7. In some cases, a nucleic acid sequence can include two or more CXCR chemokine receptor polypeptide downstream of the CAR. For example, a nucleic acid sequence encoding a CAR as used herein can include from 5′ to 3′ a promoter operably linked to a nucleic acid sequences encoding a scFv antigen binding domain capable of binding to CD19, a CD8α transmembrane domain comprising a CD8α stalk and a CD8α hinge region, a CD3zeta cytoplasmic signaling domain, a CXCR5 chemokine receptor polypeptide operably linked to the CAR (e.g., in frame) with a self-cleaving 2A sequence (e.g., a P2A, a T2A, a E2A or a F2A), and a CCR7 chemokine receptor polypeptide operably linked to the CXCR5 chemokine receptor polypeptide with a self-cleaving 2A polypeptide sequence (e.g., a P2A, a T2A, a E2A, or a F2A).

In some embodiments, a CAR can include a transmembrane domain. The transmembrane domain may be derived from a natural polypeptide, or may be artificially designed. If the transmembrane domain is derived from a natural polypeptide it can be obtained from a membrane-binding or transmembrane protein. For example, useable transmembrane domains can be from a T cell receptor alpha or beta chain, a CD3 zeta chain, CD28, CD3-epsilon, or numerous others known in the art. See, U.S. Pat. Nos. 9,670,281 and 9,834,608, both of which are incorporated by reference in their entireties. In some embodiments, the transmembrane domain is derived from CD28 or CD8. In some embodiments where the chimeric antigen receptor polypeptide includes a CD8 alpha transmembrane domain, the CD8 alpha transmembrane domain has an amino acid sequence is at least 80% (e.g., at least 85%, 90%, 95%, 99% and 100%) identical to NCBI Reference No: NP_001759 or a fragment thereof. In some embodiments where the chimeric antigen receptor polypeptide includes a CD28 transmembrane domain, the CD28 transmembrane domain has an amino acid sequence that is at least 80% (e.g., at least 85%, 90%, 95%, 99% and 100%) identical to SEQ ID NO: 277.

In some embodiments where the chimeric antigen receptor polypeptide includes a CD8 alpha transmembrane domain, the CD8 alpha transmembrane domain has an amino acid sequence that is at least 80% (e.g., at least 85%, 90%, 95%, 99% and 100%) identical to SEQ ID NO: 275 or 276.

Other transmembrane domains are known in the art and include CD16, NKG2D, NKp44, NKp46, CD27, DAP10, and DAP12 transmembrane domains.

In some cases, a nucleic acid sequence encoding a CAR as used herein can include a sequence from 5′ to 3′ a promoter operably linked to nucleic acid sequence encoding a scFv antigen binding domain capable of binding to CD11c, a CD8α transmembrane domain comprising a CD8α stalk and a CD8α hinge region, and a CD28 co-stimulatory domain (FIG. 2B). In some cases, a nucleic acid sequence encoding a CAR can include a co-stimulatory domain comprising two domains (e.g., a CD28 and a 41-BB). For example, a nucleic acid sequence encoding a CAR as used herein can include a sequence from 5′ to 3′ a promoter operably linked to nucleic acid sequence encoding a scFv antigen binding domain capable of binding to CD11c, a CD8α transmembrane domain comprising a CD8α stalk and a CD8α hinge region, and a co-stimulatory domain comprising a CD28 domain and a 41-BB domain. In some cases, a nucleic acid sequence encoding a CAR can also include chemokine receptor polypeptides. For example, a nucleic acid sequence encoding a CAR can include a promoter operably linked to a nucleic acid sequences encoding a scFv antigen binding domain capable of binding to CD11c, a CD8α transmembrane domain comprising a CD8α stalk and a CD8α hinge region, a CD28 co-stimulatory domain, and a chemokine receptor polypeptide operably linked to the CAR (e.g., in frame) with a self-cleaving 2A sequence (e.g., a P2A, a T2A, a E2A or a F2A) (FIG. 2C).

In some cases, an immuno-activatable cell can be generated by transforming the cell with nucleic acid sequences encoding two or more CARs and one or more chemokine receptor polypeptides wherein the nucleic acid sequences are on one continuous nucleic acid sequence (e.g., a polycistronic vector). For example, a nucleic acid sequence can include: (a) a nucleic acid sequences encoding a CAR comprising an antigen binding domain capable of binding to antigen on a CD11c⁺Tbet⁺ B cell, a CD8α transmembrane domain comprising a CD8α stalk and a CD8α hinge region, and a cytoplasmic signaling domain, (b) a self-cleaving polypeptide (e.g., a P2A, a T2A, a E2A or a F2A), (c) a nucleic acid sequences encoding a CAR comprising an antigen binding domain capable of binding to antigen on a CD11c⁺Tbet⁺ B cell, a CD8α transmembrane domain comprising a CD8α stalk and a CD8α hinge region, and a co-stimulatory domain, (d) a second self-cleaving polypeptide (e.g., a P2A, a T2A, a E2A or a F2A), and (e) a chemokine receptor polypeptide.

In some cases, a CAR comprising an antigen binding domain capable of binding to CD19 can include a co-stimulatory domain, and a CAR comprising an antigen binding domain capable of binding to CD11c can include a cytoplasmic signaling domain. For example, a nucleic acid sequence encoding a CAR can include a promoter operably linked to nucleic acid sequences encoding a scFv antigen binding domain capable of binding to CD19, a CD8α transmembrane domain comprising a CD8α stalk and a CD8 hinge region, a CD28 co-stimulatory domain. In such cases, the nucleic acid sequence can include a chemokine receptor polypeptide (e.g., CXCR5 or CCR7) operably linked by a 2A self-cleaving polypeptide. In another example, For example, a nucleic acid sequence encoding a CAR can include a promoter operably linked to nucleic acid sequences encoding a scFv antigen binding domain capable of binding to CD11c, a CD8α transmembrane domain comprising a CD8α stalk and a CD8α hinge region, a CD3zeta cytoplasmic signaling domain. In such cases, the nucleic acid sequence can include a chemokine receptor polypeptide (e.g., CXCR5 or CCR7) operably linked by a 2A self-cleaving polypeptide.

Exemplary CD19 antibodies or antigen binding fragments thereof are described in U.S. Pat. Nos. 11,623,956, 11,618,788, U.S. Patent Publication Number 2023/0099646, U.S. Patent Publication Number 2023/0087263, and U.S. Patent Publication Number 2023/0086030, each of which is incorporated herein by reference in its entirety. Exemplary CD20 antibodies or antigen binding fragments thereof are described in U.S. Pat. Nos. 11,623,005, 11,608,383, 11,603,411, and U.S. Patent Publication No. 2023/0056900, each of which is incorporated herein by reference in its entirety. Exemplary CD45R antibodies or antigen binding fragments thereof are described in U.S. Pat. Nos. 10,093,743, 7,160,987, and 6,010,902, each of which is incorporated herein by reference in its entirety.

In various other embodiments, the autoimmune disease can be SLE, rheumatoid arthritis, multiple sclerosis, insulin dependent diabetes mellitus, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, or polyarteritis nodosa.

As used herein, the terms “percent identity” and “identity” in the context of two or more nucleic acids or polypeptides, refer to two or more sequences that are the same or have a specified percentage of nucleotides or amino acid residues that are the same. Percent identity can be determined using sequence comparison software or algorithms or by visual inspection.

In general, percent sequence identity is calculated by determining the number of matched positions in aligned nucleic acid or polypeptide sequences, dividing the number of matched positions by the total number of aligned nucleotides or amino acids, respectively, and multiplying by 100. A matched position refers to a position in which identical nucleotides or amino acids occur at the same position in aligned sequences. The total number of aligned nucleotides or amino acids refers to the minimum number of NOTCH nucleotides or amino acids that are necessary to align the second sequence, and does not include alignment (e.g., forced alignment) with non-NOTCH sequences, such as those fused to NOTCH. The total number of aligned nucleotides or amino acids may correspond to the entire NOTC sequence or may correspond to fragments of the full-length NOTCH sequence.

Sequences can be aligned using the algorithm described by Altschul et al. (Nucleic Acids Res, 25:3389-3402, 1997) as incorporated into BLAST (basic local alignment search tool) programs, available at ncbi.nlm.nih.gov on the World Wide Web. BLAST searches or alignments can be performed to determine percent sequence identity between a NOTCH nucleic acid or polypeptide and any other sequence or portion thereof using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a NOTCH sequence and another sequence, the default parameters of the respective programs are used.

In some cases, the nucleic acid sequences encoding the CARs and the chemokine receptor polypeptides can include a nucleic acid sequence encoding a linker. This linker can provide conformational flexibility.

Also provided herein are compositions (e.g., pharmaceutical compositions) that include at least one of any of the chimeric antigen receptor polypeptides, any of the cells, or any of the nucleic acids described herein. In some embodiments, the compositions include at least one of the any of chimeric antigen receptor polypeptide described herein. In some embodiments, the compositions include any of the cells (e.g., any of the immune cells described herein including any of the immune cells produced using any of the methods described herein).

In some embodiments, the pharmaceutical compositions are formulated for different routes of administration (e.g., intravenous, subcutaneous). In some embodiments, the pharmaceutical compositions can include a pharmaceutically acceptable carrier (e.g., phosphate buffered saline).

Also provided herein are cells (e.g., any of the exemplary cells described herein) containing any of the nucleic acids described herein that encode any chimeric antigen receptor polypeptides described herein. Also provided herein are cells (e.g., any of the exemplary cells described herein) that include any of the vectors described herein that encode any chimeric antigen receptor polypeptides described herein.

In some embodiments of any of the methods described herein, the cell can be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary cells and human embryonic kidney cells (e.g., HEK293 cells).

Also provided herein are methods of treating a mammal (e.g., a human) having an autoimmune disease that includes administering to the mammal a therapeutically effective amount of a cell (e.g., an immuno-activatable cell) transformed with the chimeric antigen receptor polypeptides or nucleic acids described herein, or that includes administering any of the compositions (e.g., pharmaceutical compositions) described herein.

In some embodiments of these methods, a mammal (e.g., a human) can be identified as having an autoimmune disease. In some embodiments, these methods can result in a reduction in the number, severity, or frequency of one or more symptoms of an autoimmune disease in the mammal (e.g., as compared to the number, severity, or frequency of the one or more symptoms of the autoimmune disease in the subject prior to treatment). For example, a mammal having an autoimmune disease having been administered an immuno-activatable cell as described here can experience a reduction in inflammation or B cell autoantibody production (e.g., B cell antibody production inhibition or reduction in the number of B cells).

Any appropriate method of administration can be used to administer the immuno-activatable cells to a mammal (e.g. a human) having an autoimmune disease. Examples of methods of administration include, without limitation, parenteral administration and intravenous injection.

A pharmaceutical composition containing the immuno-activatable cells and a pharmaceutically acceptable carrier can be administered to a mammal (e.g., a human) having an autoimmune disease. For example, a pharmaceutical composition (e.g., immuno-activatable cell along with a pharmaceutically acceptable carrier) to be administered to a mammal having an autoimmune disease can be formulated in an injectable form (e.g., emulsion, solution and/or suspension).

Pharmaceutically acceptable carriers, fillers, and vehicles that can be used in a pharmaceutical composition described herein can include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Effective dosage can vary depending on the severity of the autoimmune disease, the route of administration, the age and general health condition of the subject, excipient usage, the possibility of co-usage with other therapeutic treatments, and the judgment of the treating physician. An effective amount of an immuno-activatable cell can be any amount that reduces inflammation and B cell autoantibody production (e.g., B cell antibody production inhibition or reduction in the number of B cells) within a mammal having an autoimmune disease without producing significant toxicity to the mammal. For example, an effective amount of immuno-activatable cells administered to a mammal having an autoimmune disease can be from about 1×10⁶ cells to about 1×10¹⁰ (e.g., from about 1×10⁶ to about 1×10⁹, from about 1×10⁶ to about 1×10⁸, from about 1×10⁶ to about 1×10⁷, from about 1×10⁷ to about 1×10¹⁰, from about 1×10⁷ to about 1×10⁹, from about 1×10⁷ to about 1×10⁸, from about 1×10⁸ to about 1×10¹⁰, from about 1×10⁸ to about 1×10⁹, or form about 1×10⁹ to about 1×10¹⁰). In some cases, the immuno-activatable cells can be a purified population of immune cells generated as described herein. In some cases, the purity of the population of immuno-activatable cells can be assessed using any appropriate method, including, without limitation, flow cytometry. In some cases, the population of immuno-activatable cells to be administered can include a range of purities from about 70% to about 100%, from about 70% to about 90%, from about 70% to about 80%, from about 80% to about 90%, from about 90% to about 100%, from about 80% to about 100%, from about 80% to about 90%, or from about 90% to 100%. In some cases, the dosage (e.g., number of immuno-activatable cells to be administered) can adjusted based on the level of purity of the immuno-activatable cells in the overall population of cells.

In some cases, two or more (e.g., two, three, four, five, six, or more) different immuno-activatable cells can be administered to a mammal having an autoimmune disease. For example, a percentage (e.g., from about 1.0% to about 99.0%) of the cells to be administered can include a first CAR and a second CAR and a second percentage of cells (e.g., the remaining percentage of the administered cells) can include a third CAR and a fourth CAR. In such cases, each CAR may be designed with a different antigen binding domain that binds to different antigens (e.g., different antigens on the same cell or different antigens on different cells).

The frequency of administration of an immuno-activatable cell can be any amount that reduces inflammation or B cell autoantibody production (e.g., B cell antibody production inhibition or reduction in the number of B cells) within a mammal having an autoimmune disease without producing toxicity to the mammal. In some cases, the actual frequency of administration can vary depending on various factors including, without limitation, the effective amount, duration of treatment, use of multiple treatment agents, route of administration, and severity of the condition may require an increase or decrease in frequency of administration.

An effective duration for administering a composition containing an immuno-activatable cell can be any duration that reduces inflammation or B cell autoantibody production (e.g., B cell antibody production inhibition or reduction in the number of B cells) within a mammal having an autoimmune disease without producing toxicity to the mammal. In some cases, the effective duration can vary from several days to several months. In general, the effective treatment duration for administering a composition containing an immuno-activatable cell to treat an autoimmune disease can range in duration from about one month to about five years (e.g., from about two months to about five years, from about three months to about five years, from about six months to about five years, from about eight months to about five years, from about one year to about five years, from about one month to about four years, from about one month to about three years, from about one month to about two years, from about six months to about four years, from about six months to about three years, or from about six months to about two years).

In some cases, a course of treatment and/or the severity of one or more symptoms related to autoimmune disease can be monitored. Any appropriate method can be used to determine whether the autoimmune disease is being treated. For example, immunological techniques (e.g., ELISA) can be performed to determine if the level of autoantibodies produced by the age-associated B cells present within a mammal being treated as described herein is reduced following the administration of the immuno-activatable cells. Remission and relapse of the disease can be monitored by testing for one or more markers of autoimmune disease.

Also provided herein are methods of killing, removing, and/or eliminating age-associated B cells (e.g., CD11c⁺ T-bet⁺ B cells). The methods can include administering to a subject (e.g., a mammal) a therapeutically effective amount of any of the immuno-activatable cells or compositions (e.g., pharmaceutical compositions) described herein.

Any appropriate autoimmune disease can be treated with an immuno-activatable cell as described herein. In some cases, an autoimmune disease caused by the accumulation of autoantibodies produced by age-associated B cells can be treated with an immuno-activatable cell as described herein. Examples of autoimmune diseases caused, at least in part, by age-associated B cells include, without limitation, lupus, rheumatoid arthritis, multiple sclerosis, insulin dependent diabetes mellitis, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, Crohn's disease, Celiac disease, and polyarteritis nodosa.

In some embodiments where the chimeric antigen receptor polypeptide includes an anti-CD19 scFv antigen binding domain, the anti-CD19 scFv binding domain includes a heavy chain variable region (V_{H}) and a light chain variable region (V_{L}). In some embodiments, the anti-CD19 scFv V_{H} domain can have an amino acid sequence that is at least 80% (e.g., at least 85%, 90%, 95%, 99% and 100%) identical to SEQ ID NO: 287: LKPREWKLVESGGGLVOPGGSLKLSCAASGFDFSRYWMSWVRQAPGKGLEWIGEINLD SSTINYTPSLKDKFIISRDNAKNTLYLOMSKVRSEDTALYYCARRYDAMDYWGQGTSV TVSSAKTTAPSVYPLAPVCGDTTGSSVTLGCLVKASO. In some embodiments, the anti-CD19 scFv V_{L} domain can have an amino acid sequence that is at least 80% (e.g., at least 85%, 90%, 95%, 99% and 100%) identical to SEQ ID NO: 288: ASDIWLTOSPASLAVSLGORATISCRASESVDDYGISFMNWFOOKPGQ PPKLLIYAAPNQGSGVPARFSGSGSGTDFSLNIHPMEEDDTAMYFCQQSKDVRWRHQA GDQTG. In some embodiments, any appropriate linker can be used to couple the V_{H} and V_{L} domains of the anti-CD19 scFv (e.g., GGGGSGGGGSGGGGS; SEQ ID NO: 272).

In some embodiments, where the chimeric antigen receptor polypeptide includes a CD11c scFv antigen binding domain, the anti-CD11c scFv can have an amino acid sequence that is least 80% (e.g., at least 85%, 90%, 95%, 99% and 100%) identical to the variable heavy chain and variable light chain of hamster anti-mouse CD11c mAb (cloneN418) joined by a linker (-GGGGSGGGGSGGGGS; SEQ ID NO: 272).

In some embodiments where the chimeric antigen receptor polypeptide includes a CD8 alpha transmembrane domain, the CD8 alpha transmembrane domain has an amino acid sequence is at least 80% (e.g., at least 85%, 90%, 95%, 99% and 100%) identical to NCBI Reference No: NP_001759 or a fragment thereof.

In some embodiments where the chimeric antigen receptor polypeptide includes a CD3 zeta cytoplasmic signaling domain, the CD3 zeta cytoplasmic signaling domain has an amino acid sequence that is at least 80% (e.g., at least 85%, 90%, 95%, 99% and 100%) identical to NCBI Reference No: NP_932170 or a fragment thereof that has activating or stimulatory activity.

In some embodiments where the chimeric antigen receptor polypeptide includes a CD28 co-stimulatory domain, the CD28 co-stimulatory domain is at least 80% (e.g., at least 85%, 90%, 95%, 99% and 100%) identical to SEQ ID NO: 273:

IEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAP
PRDFAAY.

As used herein, “CXCR5” refers to a C-X-C motif chemokine receptor 5 polypeptide. When preparing the immuno-activatable cell or treating a mammal with the immuno-activatable cell, “CXCR5” refers to human CXCR5. An example of a human CXCR5 polypeptide includes, without limitation, the sequence set forth in NCBI reference sequence NP_001707 (e.g., version NP_001707.1) or a fragment thereof. The CXCR5 sequence set forth in NCBI reference sequence NP_001707.1 is MNYPLTLEMDLENLEDLFWELDRLDNYNDTSL VENHLCPATE GPLMASFKAVFVPVAYSLIFLLGVIGNVLVL VILERHRQTRSSTETFLFHLAVADLLLVFI LPFAVAEGSVGWVLGTFLCKTVIALHKVNFYCSSLLLACIAVDRYLAIVHAVHAYRHRR LLSIHITCGTIWLVGFLLALPEILFAKVSQGHHNNSLPRCTFSQENQAETHAWFTSRFLYH VAGFLLPMLVMGWCYVGVVHRLRQAQRRPQRQKAVRVAILVTSIFFLCWSPYHIVIFL DTLARLKAVDNTCKLNGSLPVAITMCEFLGLAHCCLNPMLYTFAGVKFRSDLSRLLTKL GCTGPASLCQLFPSWRRSSLSESENATSLTTF (SEQ ID NO: 289)

As used herein, “CCR7” refers to C-C motif chemokine receptor 7 polypeptide. When preparing the immuno-activatable cell or treating a mammal with the immuno-activatable cell, “CCR7” refers to human CCR7. An example of a human CCR7 polypeptide includes, without limitation, NCBI reference sequence NP_001288643 (e.g., version NP_001288643.1) or a fragment thereof. The CCR7 sequence set forth in NCBI reference NP_001288643.1 is MYSIIC

(SEQ ID NO: 290)
FVGLLGNGLVVLTYIYFKRLKTMTDTYLLNLAVADILFLLTLPFWAYSA
AKSWVFGVHFCKLIFAIYKMSFFSGMLLLLCISIDRYVAIVQAVSAHRH
RARVLLISKLSCVGIWILATVLSIPELLYSDLQRSSSEQAMRCSLITEH
VEAFITIQVAQMVIGFLVPLLAMSFCYLVIIRTLLQARNFERNKAIKVI
IAVVVVFIVFQLPYNGVVLAQTVANFNITSSTCELSKQLNIAYDVTYSL
ACVRCCVNPFLYAFIGVKFRNDLFKLFKDLGCLSQEQLRQWSSCRHIRR
SSMSVEAETTTTFSP.

### EXAMPLES

### Example 1. Construction of Immuno-Activatable Cells Comprising a CD19 scFv Chimeric Antigen Receptor and a CD11c scFv Chimeric Antigen Receptor

Here, a single lentiviral vector is used to transduce two CARs and a chemokine receptor polypeptide into a T cell. FIG. 2E is an exemplary embodiment of the lentiviral vector used here. The lentiviral vector has a nucleic acid sequence that encodes for 5′ to 3′ a 3′ a CD11c CAR, a self-cleaving P2A polypeptide, a CD19 CAR, a self-cleaving T2A polypeptide, and a CXCR5 polypeptide. The CD19 CAR comprises an antigen binding domain capable of binding to CD19, a CD8α transmembrane domain comprising a CD8α stalk and a CD8α hinge region, and a CD3zeta intracellular signaling domain. The CD11c CAR comprises an antigen binding domain capable of binding to a CD11c antigen, a CD8α transmembrane domain comprising a CD8α stalk and a CD8α hinge region, and a CD28 signaling domain. The final vector is used to make lentivirus using any appropriate method (e.g., transfection of 293FT with the appropriate packaging plasmids).

Any appropriate method for isolating and culturing primary human T cell can be used. T cell placed in culture are transduced with the lentivirus. After lentiviral transduction, the expression of both the CD19 CAR and the CD11c Car can be assessed by staining with an anti-CD19 fluorescently-labeled antibody and a CD11c fluorescently-labeled antibody and analyzed using flow cytometry.

### Example 2: Treatment of Systemic Lupus Erythematosus (SLE)

A mammal suffering from SLE will be assayed to determine the presence of SLE autoantibodies prior to and after treatment with an immuno-activatable T cell generated in Example 1. Human ABCs from peripheral blood samples of SLE patients can be isolated by magnetic cell sorting system (Miltenyi et al., Cytometry 11: p231-238 (1990)). The isolated cells are then stimulated for 5-7 days with CD40L (R &D system, Cat #6420-CL) and CpG ODN 2006 (InvivoGen, Cat #tlrl-2006) in the presence or absence immuno-activatable T cell comprising a CD19 CAR, a CD11c CAR and a CXCR5 receptor. Culture supernatants will be tested for secreted Immunoglobulins (e.g., IgM, IgA and IgG) by ELISA assay and ANA autoantibodies by immunofluorescence analysis as described previously (Capolunghi et al., Rheumatology 49: p2281-89 (2010)). A patient is administered a dose of the immuno-activatable T cell comprising the CD19 CAR, the CD11c CAR and a CXCR5 receptor, in the range of 10-100 million T cells. The dose will be empirically determined depending on a number of factors, including side effects, and indications of efficacy. The modified T-cells can be administered by any method known in the art including, without limitation, intravenous, subcutaneous, intranodal, intratumoral, intrathecal, intrapleural, intraperitoneal and directly to the thymus. A single dose or multiple doses may be administered.

SEQUENCE APPENDIX

SEQ ID NO: 1 2A07 scFv
EVQLVQSGAEVKKPGESLKISCKGSGYSFTSYWIGWVRQMPGKGLEWMGIIYPGDSDTRYSPSFQGQVTISADKSISTA
YLQWSSLKASDTAMYYCARYIQGLGYYFDYWGQGTLVTVSTGGGGSGGGGSGGGGSSYELMQPPSVSVSPGQTASIT
CSGDKLGDKYVSWYQQKPGQSPVLVIYQDTKRPSGIPERFSGSNSGNTATLTISGTQAMDEADYYCQAWDSGTAIFGG
GTKVTVL
SEQ ID NO: 2 2G08 scFv
QVQLVQSGAEVKKPGESLKISCKGSGYSFTSYWIGWVRQMPGKGLEWMGIIYPGDSDTRYSPSFQGQVTISADKSISTA
YLQWSSLKASDTAMYYCARYIQGLGYYFDYWGQGTLVTVSTGGGGSGGGGSGGGGSQAGLTQPPSVSVSPGQTASIT
CSGDKLGDKYVSWYQQKPGQSPVLVIYQDSKRPSGIPERFSGSNSGNTATLTISGTQAMDEADYYCQAWDSSTVVFG
GGTKVTVL
SEQ ID NO: 3 3000000000 scFv
QLQLVQSGAEVKKPGESLKISCKGSGYSFTSYWIGWVRQMPGKGLEWMGIIYPGDSDTRYSPSFQGQVTISADKSISTA
YLQWSSLKASDTAMYYCARYIQGLGYYFDYWGQGTLVTVSTGGGGSGGGGSGGGGSQAGLTQPPSVSVSPGQTASIT
CFGDKLGHKYVSWYQQKPGQSPVLVIYQDSKRPSGIPERFSGSNSGNTATLTISGTQAMDEADYYCQAWDSSTVVFG
GGTKLTVL
SEQ ID NO: 4 ′2E03 scFv
QMQLVQSGGGVVQPGGSLRLSCAASGFTFSSYGMHWVRQAPGKGLEWVAFIRYDGSNKYYADSVKGRFTISRDNSK
NTLYLQMNSLRAEDTAVYYCAKPSRGYSRSLDYWGQGTLVTVSTGGGGSGGGGSGGGGSQPVLTQPPSVSVSPGQT
ASITCSGDKLGDKFTSWYQQRPGQSPVLVIYQDNKRPSGIPERFSGSNSGNTATLTISRVEAGDEADYYCQVWDSSSDH
WVFGGGTQLTVL
SEQ ID NO: 5 2A05 scFv
QVQLVESGAEVKKPGESLKISCKGSGYSFTSYWIGWVRQMPGKGLEWMGIIYPGDSDTRYSPSFQGQVTISADKSISTA
YLQWSSLKASDTAMYYCARLQSGWLHAFDIWGQGTMVTVSTGGGGSGGGGSGGGGSQSVLTQPPSVSVSPGQTAR
ISCSGDKLGDKYVSWYQQKPGQSPVLVIYEDSKRPSGIPERLSGSNSGNTATLTISGTQAMDEADYYCQAWDSSTVVFG
GGTKLTVL
SEQ ID NO: 6 1A01 scFv
QVQLLQSAAEVKKPGESLKISCKGSGYSFTSYWIGWVRQMPGKGLEWMGIIYPGDSDTRYSPSFQGQITISADKSISTAY
LQWSSLKASDTAMYYCARLKWSGLSHYYYYYMDVWGKGTTVTVSTGGGGSGGGGSGGGGSLSELTQDPAVSVALG
QTVRITCQGDSLRNYYASWYQQKPGQAPVLVIYGKNNRPSGIPDRFSGSRSGNTASLTITGAQAEDEADYYCNSRDSSG
NHPVVFGGGTKLTVL
SEQ ID NO: 7 1A11 scFv
QVQLLQSAAEVKKPGESLKISCKGSGYSFTSYWIGWVRQMPGKGLEWMGIIYPGDSDTRYSPSFQGQVTISADKSISTA
YLQWSSLKASDTAMYYCARLKWSGLSHYYYYYMDVWGKGTTVTVSTGGGGSGGGGSGGGGSLSELTQDPAVSVALG
QTVRITCQGDSLRSYYASWYQQKPGQAPVLVIYGKNNRPSGIPDRFSGSSSGNTASLTITGAQAEDEADYYCNSRDSSG
NHLVFGGGTKLTVL
SEQ ID NO: 8 1000000000 scFv
QVQLLQSAAEVKKPGESLKISCKGSGYSFTSYWIGWVRQMPGKGLEWMGIIYPGDSDTRYSPSFQGQVTISADKSISTA
YLQWSSLKASDTAMYYCARLKWSGLSHYYYYYMDVWGKGTTVTVSTGGGGSGGGGSGGGGSLSELTQDPAVSVALG
QTVRITCQGDSLRSYYASWYQQKPGQAPVLVIYGKNNRPSGIPDRFSGSSSGNTASLTITGAQAEDEADYYCNSRDSSG
NHVIFGGGTKLTVL
SEQ ID NO: 9 3B04 scFv
QVQLVQSGAEVKKPGESLKISCKGSGYSFTSYWIGWVRQMPGKGLEWMGIIYPGDSDTRYSPSFQGQVTISADKSISTA
YLQWSSLKASDTAMYYCARLPLGLQVGFDYWGQGTLVTVSTGGGGSGGGGSGGGGSLPVLTQPPSVSVSPGQTASIT
CSGDKLGDKYASWYQQKPGQSPVLIIYQDTKRASGIPERFSGSNSGNTATLTISGTQAVDEADYYCQAFDSSAAHFVFG
AGTKLTVL
SEQ ID NO: 10 200000 scFv
QMQLVQSGAEVKKPGESLKISCKGSGYSFTSYWIGWVRQMPGKGLEWMGIIYPGDSDTRYSPSFQGQVTISADKSIST
AYLQWSSLKASDTAMYYCARVRYSYDLNFDYWGQGTLVTVSTGGGGSGGGGSGGGGSSYELMQPPSVSVSPGQTAS
ITCSGDKLGDKYASWYQQKPGQSPVLVIYQDNKRPSGIPERFSGSNSGNTATLTISGTQAMDEADYYCQTWDSSTAVF
GGGTKVTVL
SEQ ID NO: 11 2B2 scFv
QVQLQQSGAELAKPGASVKLSCKTSGYTFTNFWMHWVKQRPGQGLEWIGYINPSSDYTKYNQKFKGKATLTADKSSS
TAYMQLSSLTYEDSAVYYCARDDYSDFGFAYWGQGTLVTVSAGGGGSGGGGSGGGGSDIQMTQSPASLSASVGETV
TITCRASENIYSFLAWYQQKQGKSPQLLVYNAKTLAEGVPSRFSGSGSGTQFSLKINSLQPEDFGSYYCQHHYGIPPTFG
GGTKLEIK
SEQ ID NO: 12 3D4-LC1 scFv
QVQLQQSGAELARPGASVKMSCKASGYTFTSYTMHWVKQRPGQGLEWIGYINPSSGYTKYNQKFKDKATLTADKSSS
TAYMQLSSLTSEDSAVYYCAREANWDDVDYWGQGTTLTVSSGGGGSGGGGSGGGGSQIVLTQSPAIMSASPGEKVT
MTCSASSSVSYMYWYLQKPGSSPRLLIYDTSNLASGVPVRFSGSGSGTSYSLTISRMEAEDAATYYCQQWSSYPLTFGA
GTKLELK
SEQ ID NO: 13 3D4-LC2 scFv
QVQLQQSGAELARPGASVKMSCKASGYTFTSYTMHWVKQRPGQGLEWIGYINPSSGYTKYNQKFKDKATLTADKSSS
TAYMQLSSLTSEDSAVYYCAREANWDDVDYWGQGTTLTVSSGGGGSGGGGSGGGGSDVLMTQTPLSLPVSLGDQA
SISCRSSQSIVHSNGNTYLEWYLQKPGQSPKLLIYKVSNRFSGVPDRFSGSGSGTDFTLKISRVEAEDLGLYYCFQGSHVPY
TFGGGTKLEIK
SEQ ID NO: 14 4A5 scFv
EFQLQQSGPELVKPGASVKISCKASGYSFTDYNMNWVKQSNGKSLEWIGVINPNYGTTSYNQKFKGKATLTVDQSSST
AYMQLNSLTSEDSAVYYCARNYYSSSYDGYFDYWGQGTTLTVSSGGGGSGGGGSGGGGSDIVLTQSPASLAVSLGQR
ATISCRASESVDNYGISFMHWYQQKPGQPPKFLIYRASNLESGIPARFSGSGSRTDFTLTINPVETDDVATYYCQQSNKD
PRTFGGGTKLEIK
SEQ ID NO: 15 400000000 scFv
QVQLQQSGPELVKPGASVKISCRASGYTFTDYYIDWVKQRPGQGLEWIGWIFPGTNSTYYNEKFKGKATLTVDKSSSTA
YMLLSSLTSEDSAVYFCARSGLRDFDYWGQGTTLTVSSGGGGSGGGGSGGGGSDIVMTQSPATLSVTPGDRVSLSCRA
SQSISDYLHWYQQKSHESPRLLIKYASQSISGIPSRFSGSGSGSDFTLSINSVEPEDVGVYYCQNGHSFPLTFGAGTKLELK
SEQ ID NO: 16 4G6 scFv
EVQLQQSGPVLVKPGASVKMSCKASGYTFTDYYMNWVKQSHGKGLEWIAVINPYSGGTSYNQKFKGKATLTVDKSSS
TAYMELSSLTSEDSAVYYCASVSSYGNYFDYWGQGTTLTVSSGGGGSGGGGSGGGGSDIVLTQSPASLAVSLGQRATIS
CRASESVSIHASHLLHWYQQKPGQPPKLLIYAASNLESGVPARFSGSGSETDFTLNIHPVEEEDAATYFCQQSIEDPWTF
GGGTKLEIK
SEQ ID NO: 17 500 scFv
QVQLQQSGPELVKPGASVKISCKASGYTFTDYYINWVKQRPGQGLEWIGWIFPGSGSTYYNEKFKGKATLTVDKSSSTV
YMLLSSLTSEDSAVYFCAREAKLGRDYFDYWGQGTTLTVSSGGGGSGGGGSGGGGSDIVMTQSHKFMSTSVGDRVSI
TCKASQDVSTAVAWCQQKPGQSPKLLIYSASYRYTGVPDRFTGSGSGTDFTFTISSVQAEDLAVYYCQQHYSTPYTFGG
GTRLEIK
SEQ ID NO: 18 5G7 scFv
EFQLQQSGPELVKPGASVKISCKASGYSFTDYNMNWVKQSNGKSLAWIGVINPNYGTTNYNQKFKGKATLTVDQSSST
AYMQLNSLTSEDSAVYYCARNYYGSTYDGYFDYWGQGTTLTVSSGGGGSGGGGSGGGGSDIVLTQSPASLAVSLGQR
ATISCRASESVDNYGISFMHWYQQKPGQPPKFLIYRASNLESGIPARFSGSGSRTDFTLTINPVETDDVATYYCQQSNKD
PRTFGGGTKLEIT
SEQ ID NO: 19 5H1 scFv
EFQLQQSGPELVKPGASVKISCKASGYSFTDYNMNWVKQSNGKSLAWIGVINPNYGTTNYNQKFKGKATLTVDQSSST
AYMQLNSLTSEDSAVYYCARNYYGSTYDGYFDYWGQGTTLTVSSGGGGSGGGGSGGGGSDIVLTQSPASLAVSLGQR
ATISCRASESVDNYGISFMHWYQQKPGQPPKFLIYRASNLESGIPARFSGSGSRTDFTLTINPVETDDVATYYCQQSNKD
PRTFGGGTKLEIT
SEQ ID NO: 20 6B2 scFv
QVQLQQSGAELMKPGASVKISCKATGYTINGYWIEWVKERPGHGLEWIGEILPGSGSTNYNEKFKGKATFTADTSSNT
AYMQLSSLTTEDSAIYYCARGMEGAMDYWGQGTSVTVSSGGGGSGGGGSGGGGSDIVMTQSHKFMSTSVGDRVSI
TCKASQDVSTAVAWYQKKPGQSPKLLIYSASYRYTGVPDRFTGSGSGTDFTFTISSVQAEDLAVYYCQQHYSTPPTFGG
GTKLEIK
SEQ ID NO: 21 6B3 scFv
QVQLQQPGAELVMPGASVRLSCKASGYTFTSYWMHWVKQRPGQGLEWIGEIDPSESYPNYNQNFKGKATLTVDKSS
STAYMQLSSLTSEDSAVYYCARSYYGRSGYAMDYWGQGTSVTVSSGGGGSGGGGSGGGGSNIVMTQSPKSTSMSVG
ERVTLNCKASENVGTYVSWYQQKPEQSPKLLIYGASNRYTGVPDRFTGSGSATDFTLTISSVQAEDLADYHCGQSYSYPP
FTFGSGTKLEIK
SEQ ID NO: 22 600000 scFv
QVQLTESGPGLVAPSQSLSITCTVSGFSLTNYIISWVRQPPGKGLEWLGVIWTGGGTNYNSALKSRLSISKDDSKSQVFLK
MNSLQTDDTARYYCARNEAVVAIFDWYFDVWGTGTTVTVSSGGGGSGGGGGGGGSDIQMTQTTSSLSASLGDRVT
ISCRASQDISNYLNWYQQKPDGAVKLLIYYTSRLHSGVPSRFSGSGSGTDFSLTISNLEQEDFATYFCQQGNTLPWTFGG
GTKLEIK
SEQ ID NO: 23 6F3 scFv
EFQLQQSGPELVKPGASVKISCKASGYSFTDYNMNWVKQSNGKSLEWIGVINPNYGTTSYNQKFKGKATLTVDQSSST
AYMQLNSLTSEDSAVYYCARNYYGNNYDGYFDYWGQGTTLTVSSGGGGSGGGGSGGGGSDIVLTQSPASLAVSLGQ
RATISCRASESVDNYGISFMHWYQQKPGQPPKFLIYRASNLESGIPARFSGSGSRTDFTLTINPVETDDVATYYCQQSNK
DPRTFGGGTKLEIT
SEQ ID NO: 24 6F4 scFv
QIQLVQSGPELKKPGETVKISCKASGYTFTMYGMSWVKQAPGKGLKWMGWINTYSGVPTYADDFKGRFAFSLETSAN
TAYLQINNLKNEDTATYFCARFPYDYDGYFDVWGTGTTVTVSSGGGGSGGGGSGGGGSDIVMTQSHKFMSTSVGDR
VSITCKASQDVGTAVAWYQQKPGQSPKLLIYWASTRHTGVPDRFTGSGSGTDFTLAISNVQSEDLADYFCHQFSSYPLT
FGAGTRLELK
SEQ ID NO: 25 9B3 scFv
EVQLQQSGPELVKPGASVKISCKASGYTFTDYYMNWVKQSHGKSLEWIGDINPNNGGTTYNQKFKGKATLTVDKSSST
AYMELRSLTSEDSAVYYCARRYYSSGYDGYFDVWGTGTTVTVSSGGGGSGGGGSGGGGSDIVLTQSPASLAVSLGQRA
TISCRASENVDNYGISFMHWYQQKPGQPPKFLIYRASNLEYGIPARFSGSGSRTDFTLTINPVETDDVATYYCQQSNKDP
LTFGAGTKLELK
SEQ ID NO: 26 15H3 scFv
EVQLQQSGPELVKPGASVKMSCKASGSTFTSYVMHWVKQKPGQGLEWIGYSNPYNDGTKYNEKFKGKATLTSDKSSS
TAYMELSSLTSEDSAVYYCARLNVLYYFDNWGQGTTLTVSSGGGGSGGGGSGGGGSDIVLTQSPASLAVSLGQRATISC
RASKSVSTSGYTYMHWYQQKPGQPPKLLIYLASNLESGVPARFSGSGSGTDFTLNIHPVEEEDAATYYCQHSRELPLTFG
AGTKLELK
SEQ ID NO: 27 8C3 scFv
QVQLQQPGTELVKPGASVKLSCKASGYTFTSYWMHWVKQRPGQGLEWIGNINPGNGGTDYNEKIKSKATLTVDKSTS
TAYMQLSSLTSEDSAVYYCARGGGYYGYDGYWYFDVWGTGTTVTVSSGGGGSGGGGSGGGGSDIVMTQAAFSIPVT
LGTSTSISCRSTKSLLHSNGITYLYWYLQKPGQSPQLLIYQMSNLASGVPDRFSSSGSGTDFTLRISRVEAEDVGVYYCAQ
NLELPWTFGGGTKLEIK
SEQ ID NO: 28 4C7 scFv
EVQLQQSGPVLVKPGPSVKISCEASGFTFTDYYMHWVKQNHGKSLEWIGLVYPYNGDTIYNQKFKGKATLTVDTSSST
AYMDLHSLTSEDSAVYYCARGANWGDYWGQGTTLTVSSGGGGSGGGGSGGGGSDVVMTQTPLSLPVSLGDQASIS
CRSSQSLVHSNGNTYLHWFLQKPGQSPKLLIYKVSNRFSGVPDRFSGSGSGTDFTLKISRVEAEDLGLYFCSQSTHVPPTF
GGGTKLEIK
SEQ ID NO: 29 4D4 scFv
QVQLQQPGTELVKPGASVKLSCKASGYTFTSYWMHWVKQRPGQGLEWIGNISPSNGGTNYNENFKSKATLTVDKSSS
TAYMQLSSLTSEDSAVYYCATYYVDYWGQGTTLTVSSGGGGSGGGGSGGGGSDVVMTQTPLTLSVTIGQPASISCKST
QSLLDSDGKTYLNWFLQRPGQSPKRLIYLVSKLDSGVPDRFTGSGSGTDFTLKISRVEAEDLGLYYCWQGTHFPQTFGG
GTKLEIK
SEQ ID NO: 30 6A3 scFv
QVTLKESGPGILQSSQTLSLTCSFSGFSLSTSGMGVSWIRQPSGKGLEWLAHIYWDDDKRYNPSLKSRLTISKDTSRNQV
FLKITSVDTADTATYYCARRVYGYDPYAMNYWGPGTSVTVSSGGGGSGGGGSGGGGSQIVLTQSPALMSASPGEKVT
MTCSASSSVSYMYWYQQKPRSSPKPWIYLTSTLASGVPARFSGSGSGTSYSLTISSMEAEDAATYYCQQWSSNPYTFG
GGTKLEIK
SEQ ID NO: 31 8B8 scFv
EVQLQQSGPELVKPGASVKISCKASGYTFTDYYMNWVKQSHGKSLEWIGDINPNNGGTSYNQKFKGKATLTVDKSSST
AYMELRSLTSEDSAVYYCAPHYYGSSYDWYFDVWGTGTTVTVSSGGGGSGGGGSGGGGSDIVLTQSPASLAVSLGQR
ATISCRASESVDNYGISFMHWYQQKPGQPPKLLIYRASNLESGIPARFSGSGSRTDFTLTINPVETDDVATYYCQQSNKD
PLTFGAGTKLELK
SEQ ID NO: 32 9B1 scFv
EVQLQQSGPELVKPGASVKIPCKASGYTFTDYNMDWVKQSHGKSLEWIGDINPNNGGTIYNQKFKGKATLTVDKSSST
AYMELRSLTSEDTAVYYCAREDDSRYWYFDVWGTGTTVTVSSGGGGSGGGGSGGGGSDIVMTQSHKFMSTSVGDR
VSITCKASQDVGTAVAWYQQKPGQSPKLLIYWASTRHTGVPDRFTGSGSGTDFTLTISNVQSEDLADYFCQQYSSYPYT
FGGGTKLEIK
SEQ ID NO: 33 9B2 scFv
QIQFVQSGPELKKPGETVKISCKASVYTFTEYPMHWVKQAPGKGFKWMGWINTYSGEPTYADDFKGRFAFSLETSAST
AYLQINNLKNEDTASYFCAREGWLDAMDYWGQGTSVTVSSGGGGSGGGGSGGGGSDIVMTQSQKFMSTIVGDRVS
ITCKASQNVGTAVAWYQQKPGQSPKLLIYSASNRYTGVPDRFTGSGSGTDFTLTISNMQSEDLADYFCQQYSSYTWTF
GGGTKLEIK
SEQ ID NO: 34 9C1 scFv
QVQLKQSGPGLVQPSQSLSITCTVSGFSLTTFGVHWVRQSPGKGLEWLGVIWSGGSTDYNAAFISRLSISKDNSKSQVF
FKMNSLQVDDTAIYYCAPRLGLRAYWGQGTLVTVSAGGGGSGGGGSGGGGSDIKMTQSPSSMYASLGERVTITCKAS
QDINSYLSWFQQKPGKSPKTLIYRANRLVDGVPSRFSGSGSGQDYSLTISSLEYEDMGIYYCLQYDEFPYTFGGGTKLEVK
SEQ ID NO: 35 9G1 scFv
QVQLKQSGPGLVQPSQSLSITCTVSGFSLTSYGVHWVRQSPGKGLEWLGVIWSGGTTDYNAAFISRLSISKDNSKSQVF
FKMNSLQADDTAIYYCARMGGTGYFDVWGTGTTVTVSSGGGGSGGGGSGGGGSNIVMTQSPKSMSMSVGERVTLS
CKAGENVGPYVSWYQQKPEQSPKLLIYGASNRFTGVPDRFTGSGSATDFTLTISSVQAEDLADYHCGQSYSYPFTFGSG
TKLEIK
SEQ ID NO: 36 3G6 scFv
QVQLKQSGPGLVQPSQSLSITCTVSGFSLTSYGVHWVRQSPGKGLEWLGVIWSGGTTDYNAAFISRLSISKDNSKSQVF
FKMNSLQADDTAIYYCARMGGTGYFDVWGTGTTVTVSSGGGGSGGGGGGGGSDIVMTQAAFSNPVTLGTSASISC
RSSKSLLHSNGITYLYWYLQKPGLSPQLLIYHMSNLASGVPDRFSSSGSGTDFTLRISRVEAEDVGVYYCAQNLELPWTFG
GGTKLEIK
SEQ ID NO: 37 3.9 VH-VL full scFv
QVQLVQSGAEVKKPGASVKVSCKASGYTFTDYNMHWVRQAPGQGLEWMGYIYPYNGGTAYNQKFKNRVTMTRDT
STSTVYMELSSLRSEDTAVYYCARGLDVMDYWGQGTLVTVSSGGGGSGGGGSGGGGSAIQLTQSPSSLSASVGDRVTI
TCRASENIYSYLAWYQQKPGKAPKLLIYNAKILAEGVPSRFSGSGSGTDFTLTISSLQPEDFATYYCQHHYVSPRTFGQGT
KLEIK
SEQ ID NO: 38 VL-VH full scFV
DIQMTQSPSSLSASVGDRVTITCRASENIYSYLAWYQQKPGKAPKLLIYNAKILAEGVPSRFSGSGSGTDFTLTISSLQPED
FATYYCQHHYVSPRTFGQGTKLEIKGGGGSGGGGSGGGGSQVQLVQSGAEVKKPGASVKVSCKVSGYTFTDYNMHW
VRQAPGKGLEWMGYIYPYNGGTAYNQKFKNRVTMTEDTSTDTAYMELSSLRSEDTAVYYCARGLDVMDYWGQGTL
VTVSS
SEQ ID NO: 39 2A07 VH Domain
EVQLVQSGAEVKKPGESLKISCKGSGYSFTSYWIGWVRQMPGKGLEWMGIIYPGDSDTRYSPSFQGQVTISADKSIST
AYLQWSSLKASDTAMYYCARYIQGLGYYFDYWGQGTLVTVST
SEQ ID NO: 40 2G08 VH Domain
QVQLVQSGAEVKKPGESLKISCKGSGYSFTSYWIGWVRQMPGKGLEWMGIIYPGDSDTRYSPSFQGQVTISADKSIST
AYLQWSSLKASDTAMYYCARYIQGLGYYFDYWGQGTLVTVST
SEQ ID NO: 41 3000000000 VH Domain
QLQLVQSGAEVKKPGESLKISCKGSGYSFTSYWIGWVRQMPGKGLEWMGIIYPGDSDTRYSPSFQGQVTISADKSIST
AYLQWSSLKASDTAMYYCARYIQGLGYYFDYWGQGTLVTVST
SEQ ID NO: 42 ′2E03 VH Domain
QMQLVQSGGGVVQPGGSLRLSCAASGFTFSSYGMHWVRQAPGKGLEWVAFIRYDGSNKYYADSVKGRFTISRDNS
KNTLYLQMNSLRAEDTAVYYCAKPSRGYSRSLDYWGQGTLVTVST
SEQ ID NO: 43 2A05 VH Domain
QVQLVESGAEVKKPGESLKISCKGSGYSFTSYWIGWVRQMPGKGLEWMGIIYPGDSDTRYSPSFQGQVTISADKSIST
AYLQWSSLKASDTAMYYCARLQSGWLHAFDIWGQGTMVTVST
SEQ ID NO: 44 1A01 VH Domain
QVQLLQSAAEVKKPGESLKISCKGSGYSFTSYWIGWVRQMPGKGLEWMGIIYPGDSDTRYSPSFQGQITISADKSISTA
YLQWSSLKASDTAMYYCARLKWSGLSHYYYYYMDVWGKGTTVTVST
SEQ ID NO: 45 1A11 VH Domain
QVQLLQSAAEVKKPGESLKISCKGSGYSFTSYWIGWVRQMPGKGLEWMGIIYPGDSDTRYSPSFQGQVTISADKSIST
AYLQWSSLKASDTAMYYCARLKWSGLSHYYYYYMDVWGKGTTVTVST
SEQ ID NO: 46 1000000000 VH Domain
QVQLLQSAAEVKKPGESLKISCKGSGYSFTSYWIGWVRQMPGKGLEWMGIIYPGDSDTRYSPSFQGQVTISADKSIST
AYLQWSSLKASDTAMYYCARLKWSGLSHYYYYYMDVWGKGTTVTVST
SEQ ID NO: 47 3B04 VH Domain
QVQLVQSGAEVKKPGESLKISCKGSGYSFTSYWIGWVRQMPGKGLEWMGIIYPGDSDTRYSPSFQGQVTISADKSIST
AYLQWSSLKASDTAMYYCARLPLGLQVGFDYWGQGTLVTVST
SEQ ID NO: 48 200000 VH Domain
QMQLVQSGAEVKKPGESLKISCKGSGYSFTSYWIGWVRQMPGKGLEWMGIIYPGDSDTRYSPSFQGQVTISADKSIS
TAYLQWSSLKASDTAMYYCARVRYSYDLNFDYWGQGTLVTVST
SEQ ID NO: 49 2B2 VH Domain
QVQLQQSGAELAKPGASVKLSCKTSGYTFTNFWMHWVKQRPGQGLEWIGYINPSSDYTKYNQKFKGKATLTADKSS
STAYMQLSSLTYEDSAVYYCARDDYSDFGFAYWGQGTLVTVSA
SEQ ID NO: 50 3D4-LC1 VH Domain
QVQLQQSGAELARPGASVKMSCKASGYTFTSYTMHWVKQRPGQGLEWIGYINPSSGYTKYNQKFKDKATLTADKSS
STAYMQLSSLTSEDSAVYYCAREANWDDVDYWGQGTTLTVSS
SEQ ID NO: 51 3D4-LC2 VH Domain
QVQLQQSGAELARPGASVKMSCKASGYTFTSYTMHWVKQRPGQGLEWIGYINPSSGYTKYNQKFKDKATLTADKSS
STAYMQLSSLTSEDSAVYYCAREANWDDVDYWGQGTTLTVSS
SEQ ID NO: 52 4A5 VH Domain
EFQLQQSGPELVKPGASVKISCKASGYSFTDYNMNWVKQSNGKSLEWIGVINPNYGTTSYNQKFKGKATLTVDQSSS
TAYMQLNSLTSEDSAVYYCARNYYSSSYDGYFDYWGQGTTLTVSS
SEQ ID NO: 53 400000000 VH Domain
QVQLQQSGPELVKPGASVKISCRASGYTFTDYYIDWVKQRPGQGLEWIGWIFPGTNSTYYNEKFKGKATLTVDKSSST
AYMLLSSLTSEDSAVYFCARSGLRDFDYWGQGTTLTVSS
SEQ ID NO: 54 4G6 VH Domain
EVQLQQSGPVLVKPGASVKMSCKASGYTFTDYYMNWVKQSHGKGLEWIAVINPYSGGTSYNQKFKGKATLTVDKSS
STAYMELSSLTSEDSAVYYCASVSSYGNYFDYWGQGTTLTVSS
SEQ ID NO: 55 500 VH Domain
QVQLQQSGPELVKPGASVKISCKASGYTFTDYYINWVKQRPGQGLEWIGWIFPGSGSTYYNEKFKGKATLTVDKSSST
VYMLLSSLTSEDSAVYFCAREAKLGRDYFDYWGQGTTLTVSS
SEQ ID NO: 56 5G7 VH Domain
QIQLVQSGPELKKPGETVKISCKASGYTFTTYGMSWVKQAPGKGLKWMGWINTYSGVPTYADDFKGRFAFSLETSAS
TAYLQINNLKNEDTTTYFCARFPYDFDGYFDVWGTGTAVTVSS
SEQ ID NO: 57 5H1 VH Domain
EFQLQQSGPELVKPGASVKISCKASGYSFTDYNMNWVKQSNGKSLAWIGVINPNYGTTNYNQKFKGKATLTVDQSSS
TAYMQLNSLTSEDSAVYYCARNYYGSTYDGYFDYWGQGTTLTVSS
SEQ ID NO: 58 6B2 VH Domain
QVQLQQSGAELMKPGASVKISCKATGYTINGYWIEWVKERPGHGLEWIGEILPGSGSTNYNEKFKGKATFTADTSSNT
AYMQLSSLTTEDSAIYYCARGMEGAMDYWGQGTSVTVSS
SEQ ID NO: 59 6B3 VH Domain
QVQLQQPGAELVMPGASVRLSCKASGYTFTSYWMHWVKQRPGQGLEWIGEIDPSESYPNYNQNFKGKATLTVDKS
SSTAYMQLSSLTSEDSAVYYCARSYYGRSGYAMDYWGQGTSVTVSS
SEQ ID NO: 60 600000 VH Domain
QVQLTESGPGLVAPSQSLSITCTVSGFSLTNYIISWVRQPPGKGLEWLGVIWTGGGTNYNSALKSRLSISKDDSKSQVFL
KMNSLQTDDTARYYCARNEAVVAIFDWYFDVWGTGTTVTVSS
SEQ ID NO: 61 6F3 VH Domain
EFQLQQSGPELVKPGASVKISCKASGYSFTDYNMNWVKQSNGKSLEWIGVINPNYGTTSYNQKFKGKATLTVDQSSS
TAYMQLNSLTSEDSAVYYCARNYYGNNYDGYFDYWGQGTTLTVSS
SEQ ID NO: 62 6F4 VH Domain
QIQLVQSGPELKKPGETVKISCKASGYTFTMYGMSWVKQAPGKGLKWMGWINTYSGVPTYADDFKGRFAFSLETSA
NTAYLQINNLKNEDTATYFCARFPYDYDGYFDVWGTGTTVTVSS
SEQ ID NO: 63 9B3 VH Domain
EVQLQQSGPELVKPGASVKISCKASGYTFTDYYMNWVKQSHGKSLEWIGDINPNNGGTTYNQKFKGKATLTVDKSSS
TAYMELRSLTSEDSAVYYCARRYYSSGYDGYFDVWGTGTTVTVSS
SEQ ID NO: 64 15H3 VH Domain
EVQLQQSGPELVKPGASVKMSCKASGSTFTSYVMHWVKQKPGQGLEWIGYSNPYNDGTKYNEKFKGKATLTSDKSS
STAYMELSSLTSEDSAVYYCARLNVLYYFDNWGQGTTLTVSS
SEQ ID NO: 65 8C3 VH Domain
QVQLQQPGTELVKPGASVKLSCKASGYTFTSYWMHWVKQRPGQGLEWIGNINPGNGGTDYNEKIKSKATLTVDKST
STAYMQLSSLTSEDSAVYYCARGGGYYGYDGYWYFDVWGTGTTVTVSS
SEQ ID NO: 66 4C7 VH Domain
EVQLQQSGPVLVKPGPSVKISCEASGFTFTDYYMHWVKQNHGKSLEWIGLVYPYNGDTIYNQKFKGKATLTVDTSSST
AYMDLHSLTSEDSAVYYCARGANWGDYWGQGTTLTVSS
SEQ ID NO: 67 4D4 VH Domain
QVQLQQPGTELVKPGASVKLSCKASGYTFTSYWMHWVKQRPGQGLEWIGNISPSNGGTNYNENFKSKATLTVDKSS
STAYMQLSSLTSEDSAVYYCATYYVDYWGQGTTLTVSS
SEQ ID NO: 68 6A3 VH Domain
QVTLKESGPGILQSSQTLSLTCSFSGFSLSTSGMGVSWIRQPSGKGLEWLAHIYWDDDKRYNPSLKSRLTISKDTSRNQ
VFLKITSVDTADTATYYCARRVYGYDPYAMNYWGPGTSVTVSS
SEQ ID NO: 69 8B8 VH Domain
EVQLQQSGPELVKPGASVKISCKASGYTFTDYYMNWVKQSHGKSLEWIGDINPNNGGTSYNQKFKGKATLTVDKSSS
TAYMELRSLTSEDSAVYYCAPHYYGSSYDWYFDVWGTGTTVTVSS
SEQ ID NO: 70 9B1 VH Domain
EVQLQQSGPELVKPGASVKIPCKASGYTFTDYNMDWVKQSHGKSLEWIGDINPNNGGTIYNQKFKGKATLTVDKSSS
TAYMELRSLTSEDTAVYYCAREDDSRYWYFDVWGTGTTVTVSS
SEQ ID NO: 71 9B2 VH Domain
QIQFVQSGPELKKPGETVKISCKASVYTFTEYPMHWVKQAPGKGFKWMGWINTYSGEPTYADDFKGRFAFSLETSAS
TAYLQINNLKNEDTASYFCAREGWLDAMDYWGQGTSVTVSS
SEQ ID NO: 72 9C1 VH Domain
QVQLKQSGPGLVQPSQSLSITCTVSGFSLTTFGVHWVRQSPGKGLEWLGVIWSGGSTDYNAAFISRLSISKDNSKSQVF
FKMNSLQVDDTAIYYCAPRLGLRAYWGQGTLVTVSA
SEQ ID NO: 73 9G1 VH Domain
QVQLKQSGPGLVQPSQSLSITCTVSGFSLTSYGVHWVRQSPGKGLEWLGVIWSGGTTDYNAAFISRLSISKDNSKSQV
FFKMNSLQADDTAIYYCARMGGTGYFDVWGTGTTVTVSS
SEQ ID NO: 74 3G6 VH Domain
QVQLKQSGPGLVQPSQSLSITCTVSGFSLTSYGVHWVRQSPGKGLEWLGVIWSGGTTDYNAAFISRLSISKDNSKSQV
FFKMNSLQADDTAIYYCARMGGTGYFDVWGTGTTVTVSS
SEQ ID NO: 75 3.9 VH-VL VH Domain
QVQLVQSGAEVKKPGASVKVSCKASGYTFTDYNMHWVRQAPGQGLEWMGYIYPYNGGTAYNQKFKNRVTMTRD
TSTSTVYMELSSLRSEDTAVYYCARGLDVMDYWGQGTLVTVSS
SEQ ID NO: 76 3.9 VL-VH VH Domain
QVQLVQSGAEVKKPGASVKVSCKVSGYTFTDYNMHWVRQAPGKGLEWMGYIYPYNGGTAYNQKFKNRVTMTEDT
STDTAYMELSSLRSEDTAVYYCARGLDVMDYWGQGTLVTVSS
SEQ ID NO: 77 2A07 VL Domain
SYELMQPPSVSVSPGQTASITCSGDKLGDKYVSWYQQKPGQSPVLVIYQDTKRPSGIPERFSGSNSGNTATLTISGTQA
MDEADYYCQAWDSGTAIFGGGTKVTVL
SEQ ID NO: 78 2G08 VL Domain
QAGLTQPPSVSVSPGQTASITCSGDKLGDKYVSWYQQKPGQSPVLVIYQDSKRPSGIPERFSGSNSGNTATLTISGTQA
MDEADYYCQAWDSSTVVFGGGTKVTVL
SEQ ID NO: 79 3000000000 VL Domain
QAGLTQPPSVSVSPGQTASITCFGDKLGHKYVSWYQQKPGQSPVLVIYQDSKRPSGIPERFSGSNSGNTATLTISGTQA
MDEADYYCQAWDSSTVVFGGGTKLTVL
SEQ ID NO: 80 ′2E03 VL Domain
QPVLTQPPSVSVSPGQTASITCSGDKLGDKFTSWYQQRPGQSPVLVIYQDNKRPSGIPERFSGSNSGNTATLTISRVEA
GDEADYYCQVWDSSSDHWVFGGGTQLTVL
SEQ ID NO: 81 2A05 VL Domain
QSVLTQPPSVSVSPGQTARISCSGDKLGDKYVSWYQQKPGQSPVLVIYEDSKRPSGIPERLSGSNSGNTATLTISGTQA
MDEADYYCQAWDSSTVVFGGGTKLTVL
SEQ ID NO: 82 1A01 VL Domain
LSELTQDPAVSVALGQTVRITCQGDSLRNYYASWYQQKPGQAPVLVIYGKNNRPSGIPDRFSGSRSGNTASLTITGAQ
AEDEADYYCNSRDSSGNHPVVFGGGTKLTVL
SEQ ID NO: 83 1A11 VL Domain
LSELTQDPAVSVALGQTVRITCQGDSLRSYYASWYQQKPGQAPVLVIYGKNNRPSGIPDRFSGSSSGNTASLTITGAQA
EDEADYYCNSRDSSGNHLVFGGGTKLTVL
SEQ ID NO: 84 1000000000 VL Domain
LSELTQDPAVSVALGQTVRITCQGDSLRSYYASWYQQKPGQAPVLVIYGKNNRPSGIPDRFSGSSSGNTASLTITGAQA
EDEADYYCNSRDSSGNHVIFGGGTKLTVL
SEQ ID NO: 85 3B04 VL Domain
LPVLTQPPSVSVSPGQTASITCSGDKLGDKYASWYQQKPGQSPVLIIYQDTKRASGIPERFSGSNSGNTATLTISGTQAV
DEADYYCQAFDSSAAHFVFGAGTKLTVL
SEQ ID NO: 86 200000 VL Domain
SYELMQPPSVSVSPGQTASITCSGDKLGDKYASWYQQKPGQSPVLVIYQDNKRPSGIPERFSGSNSGNTATLTISGTQA
MDEADYYCQTWDSSTAVFGGGTKVTVL
SEQ ID NO: 87 2B2 VL Domain
DIQMTQSPASLSASVGETVTITCRASENIYSFLAWYQQKQGKSPQLLVYNAKTLAEGVPSRFSGSGSGTQFSLKINSLQP
EDFGSYYCQHHYGIPPTFGGGTKLEIK
SEQ ID NO: 88 3D4-LC1 VL Domain
QIVLTQSPAIMSASPGEKVTMTCSASSSVSYMYWYLQKPGSSPRLLIYDTSNLASGVPVRFSGSGSGTSYSLTISRMEAE
DAATYYCQQWSSYPLTFGAGTKLELK
SEQ ID NO: 89 3D4-LC2 VL Domain
DVLMTQTPLSLPVSLGDQASISCRSSQSIVHSNGNTYLEWYLQKPGQSPKLLIYKVSNRFSGVPDRFSGSGSGTDFTLKI
SRVEAEDLGLYYCFQGSHVPYTFGGGTKLEIK
SEQ ID NO: 90 4A5 VL Domain
DIVLTQSPASLAVSLGQRATISCRASESVDNYGISFMHWYQQKPGQPPKFLIYRASNLESGIPARFSGSGSRTDFTLTINP
VETDDVATYYCQQSNKDPRTFGGGTKLEIK
SEQ ID NO: 91 400000000 VL Domain
DIVMTQSPATLSVTPGDRVSLSCRASQSISDYLHWYQQKSHESPRLLIKYASQSISGIPSRFSGSGSGSDFTLSINSVEPED
VGVYYCQNGHSFPLTFGAGTKLELK
SEQ ID NO: 92 4G6 VL Domain
DIVLTQSPASLAVSLGQRATISCRASESVSIHASHLLHWYQQKPGQPPKLLIYAASNLESGVPARFSGSGSETDFTLNIHP
VEEEDAATYFCQQSIEDPWTFGGGTKLEIK
SEQ ID NO: 93 500 VL Domain
DIVMTQSHKFMSTSVGDRVSITCKASQDVSTAVAWCQQKPGQSPKLLIYSASYRYTGVPDRFTGSGSGTDFTFTISSV
QAEDLAVYYCQQHYSTPYTFGGGTRLEIK
SEQ ID NO: 94 5G7 VL Domain
DIVMTQSHKFMSTSVGDRVSITCKASQDVGTAVAWYQEKPGQSPKILIYWASTRHTGVPDRFTGSGSGTDFTLTISNV
QSEDLADYFCQQYSSYPLTFGAGTKLELK
SEQ ID NO: 95 5H1 VL Domain
DIVLTQSPASLAVSLGQRATISCRASESVDNYGISFMHWYQQKPGQPPKFLIYRASNLESGIPARFSGSGSRTDFTLTINP
VETDDVATYYCQQSNKDPRTFGGGTKLEIT
SEQ ID NO: 96 6B2 VL Domain
DIVMTQSHKFMSTSVGDRVSITCKASQDVSTAVAWYQKKPGQSPKLLIYSASYRYTGVPDRFTGSGSGTDFTFTISSVQ
AEDLAVYYCQQHYSTPPTFGGGTKLEIK
SEQ ID NO: 97 6B3 VL Domain
NIVMTQSPKSTSMSVGERVTLNCKASENVGTYVSWYQQKPEQSPKLLIYGASNRYTGVPDRFTGSGSATDFTLTISSV
QAEDLADYHCGQSYSYPPFTFGSGTKLEIK
SEQ ID NO: 98 600000 VL Domain
DIQMTQTTSSLSASLGDRVTISCRASQDISNYLNWYQQKPDGAVKLLIYYTSRLHSGVPSRFSGSGSGTDFSLTISNLEQE
DFATYFCQQGNTLPWTFGGGTKLEIK
SEQ ID NO: 99 6F3 VL Domain
DIVLTQSPASLAVSLGQRATISCRASESVDNYGISFMHWYQQKPGQPPKFLIYRASNLESGIPARFSGSGSRTDFTLTINP
VETDDVATYYCQQSNKDPRTFGGGTKLEIT
SEQ ID NO: 100 6F4 VL Domain
DIVMTQSHKFMSTSVGDRVSITCKASQDVGTAVAWYQQKPGQSPKLLIYWASTRHTGVPDRFTGSGSGTDFTLAISN
VQSEDLADYFCHQFSSYPLTFGAGTRLELK
SEQ ID NO: 101 9B3 VL Domain
DIVLTQSPASLAVSLGQRATISCRASENVDNYGISFMHWYQQKPGQPPKFLIYRASNLEYGIPARFSGSGSRTDFTLTIN
PVETDDVATYYCQQSNKDPLTFGAGTKLELK
SEQ ID NO: 102 15H3 VL Domain
DIVLTQSPASLAVSLGQRATISCRASKSVSTSGYTYMHWYQQKPGQPPKLLIYLASNLESGVPARFSGSGSGTDFTLNIH
PVEEEDAATYYCQHSRELPLTFGAGTKLELK
SEQ ID NO: 103 8C3 VL Domain
DIVMTQAAFSIPVTLGTSTSISCRSTKSLLHSNGITYLYWYLQKPGQSPQLLIYQMSNLASGVPDRESSSGSGTDFTLRISR
VEAEDVGVYYCAQNLELPWTFGGGTKLEIK
SEQ ID NO: 104 4C7 VL Domain
DVVMTQTPLSLPVSLGDQASISCRSSQSLVHSNGNTYLHWFLQKPGQSPKLLIYKVSNRFSGVPDRFSGSGSGTDFTLKI
SRVEAEDLGLYFCSQSTHVPPTFGGGTKLEIK
SEQ ID NO: 105 4D4 VL Domain
DVVMTQTPLTLSVTIGQPASISCKSTQSLLDSDGKTYLNWFLQRPGQSPKRLIYLVSKLDSGVPDRFTGSGSGTDFTLKIS
RVEAEDLGLYYCWQGTHFPQTFGGGTKLEIK
SEQ ID NO: 106 6A3 VL Domain
QIVLTQSPALMSASPGEKVTMTCSASSSVSYMYWYQQKPRSSPKPWIYLTSTLASGVPARFSGSGSGTSYSLTISSMEA
EDAATYYCQQWSSNPYTFGGGTKLEIK
SEQ ID NO: 107 8B8 VL Domain
DIVLTQSPASLAVSLGQRATISCRASESVDNYGISFMHWYQQKPGQPPKLLIYRASNLESGIPARFSGSGSRTDFTLTINP
VETDDVATYYCQQSNKDPLTFGAGTKLELK
SEQ ID NO: 108 9B1 VL Domain
DIVMTQSHKFMSTSVGDRVSITCKASQDVGTAVAWYQQKPGQSPKLLIYWASTRHTGVPDRFTGSGSGTDFTLTISN
VQSEDLADYFCQQYSSYPYTFGGGTKLEIK
SEQ ID NO: 109 9B2 VL Domain
DIVMTQSQKFMSTIVGDRVSITCKASQNVGTAVAWYQQKPGQSPKLLIYSASNRYTGVPDRFTGSGSGTDFTLTISNM
QSEDLADYFCQQYSSYTWTFGGGTKLEIK
SEQ ID NO: 110 9C1 VL Domain
DIKMTQSPSSMYASLGERVTITCKASQDINSYLSWFQQKPGKSPKTLIYRANRLVDGVPSRFSGSGSGQDYSLTISSLEY
EDMGIYYCLQYDEFPYTFGGGTKLEVK
SEQ ID NO: 111 9G1 VL Domain
NIVMTQSPKSMSMSVGERVTLSCKAGENVGPYVSWYQQKPEQSPKLLIYGASNRFTGVPDRFTGSGSATDFTLTISSV
QAEDLADYHCGQSYSYPFTFGSGTKLEIK
SEQ ID NO: 112 3G6 VL Domain
DIVMTQAAFSNPVTLGTSASISCRSSKSLLHSNGITYLYWYLQKPGLSPQLLIYHMSNLASGVPDRESSSGSGTDFTLRIS
RVEAEDVGVYYCAQNLELPWTFGGGTKLEIK
SEQ ID NO: 113 3.9 VH-VL VL Domain
AIQLTQSPSSLSASVGDRVTITCRASENIYSYLAWYQQKPGKAPKLLIYNAKILAEGVPSRFSGSGSGTDFTLTISSLQPED
FATYYCQHHYVSPRTFGQGTKLEIK
SEQ ID NO: 114 3.9 VL-VH VL Domain
DIQMTQSPSSLSASVGDRVTITCRASENIYSYLAWYQQKPGKAPKLLIYNAKILAEGVPSRFSGSGSGTDFTLTISSLQPE
DFATYYCQHHYVSPRTFGQGTKLEIK
SEQ ID NO: 115 CDR H1 #1 GYSFTSYWIG
SEQ ID NO: 116 CDR H1 #2 GFTFSSYGMH
SEQ ID NO: 117 CDR H1 #3 GYTFTNFWMH
SEQ ID NO: 118 CDR H1 #4 GYTFTSYTMH
SEQ ID NO: 119 CDR H1 #5 GYSFTDYNMN
SEQ ID NO: 120 CDR H1 #6 GYTFTDYYID
SEQ ID NO: 121 CDR H1 #7 GYTFTDYYMN
SEQ ID NO: 122 CDR H1 #8 GYTFTDYYIN
SEQ ID NO: 123 CDR H1 #9 GYTFTTYGMSWV
SEQ ID NO: 124 CDR H1 #10 GYTINGY
SEQ ID NO: 125 CDR H1 #11 GYTFTSYWMH
SEQ ID NO: 126 CDR H1 #12 GFSLTNYIIS
SEQ ID NO: 127 CDR H1 #13 GYTFTMYGMSWV
SEQ ID NO: 128 CDR H1 #14 GSTFTSYVMH
SEQ ID NO: 129 CDR H1 #15 GFTFTDYYMH
SEQ ID NO: 130 CDR H1 #16 GFSLSTSGMGVS
SEQ ID NO: 131 CDR H1 #17 GYTFTDYNMD
SEQ ID NO: 132 CDR H1 #18 VYTFTEYPMHWV
SEQ ID NO: 133 CDR H1 #19 GFSLTTFGVH
SEQ ID NO: 134 CDR H1 #20 GFSLTSYGVH
SEQ ID NO: 135 CDR H1 #21 GYTFTDYNMH
SEQ ID NO: 136 CDR H2 #1 IIYPGDSDTRYSPSFQG
SEQ ID NO: 137 CDR H2 #2 FIRYDGSNKYYADSVKG
SEQ ID NO: 138 CDR H2 #3 INPSSDYTKYNQKFKG
SEQ ID NO: 139 CDR H2 #4 INPSSGYTKYNQKFKD
SEQ ID NO: 140 CDR H2 #5 INPNYGTTSYNQKFKG
SEQ ID NO: 141 CDR H2 #6 IFPGTNSTYYNEKFKG
SEQ ID NO: 142 CDR H2 #7 INPYSGGTSYNQKFKG
SEQ ID NO: 143 CDR H2 #8 IFPGSGSTYYNEKFKG
SEQ ID NO: 144 CDR H2 #9 WINTYSGVPTYADDFK
SEQ ID NO: 145 CDR H2 #10 INPNYGTTNYNQKFKG
SEQ ID NO: 146 CDR H2 #11 ILPGSGSTNYNEKFKG
SEQ ID NO: 147 CDR H2 #12 IDPSESYPNYNQNFKG
SEQ ID NO: 148 CDR H2 #13 IWTGGGTNYNSALKS
SEQ ID NO: 149 CDR H2 #14 INPNNGGTTYNQKFKG
SEQ ID NO: 150 CDR H2 #15 SNPYNDGTKYNEKFKG
SEQ ID NO: 151 CDR H2 #16 INPGNGGTDYNEKIKS
SEQ ID NO: 152 CDR H2 #17 VYPYNGDTIYNQKFKG
SEQ ID NO: 153 CDR H2 #18 ISPSNGGTNYNENFKS
SEQ ID NO: 154 CDR H2 #19 IYWDDDKRYNPSLKS
SEQ ID NO: 155 CDR H2 #20 INPNNGGTSYNQKFKG
SEQ ID NO: 156 CDR H2 #21 INPNNGGTIYNQKFKG
SEQ ID NO: 157 CDR H2 #22 WINTYSGEPTYADDFK
SEQ ID NO: 158 CDR H2 #23 IWSGGSTDYNAAFIS
SEQ ID NO: 159 CDR H2 #24 IWSGGTTDYNAAFIS
SEQ ID NO: 160 CDR H2 #25 YIYPYNGGTAYNQKFKN
SEQ ID NO: 161 CDR H3 #1 ARYIQGLGYYFDY
SEQ ID NO: 162 CDR H3 #2 AKPSRGYSRSLDY
SEQ ID NO: 163 CDR H3 #3 ARLQSGWLHAFDI
SEQ ID NO: 164 CDR H3 #4 ARLKWSGLSHYYYYYMDV
SEQ ID NO: 165 CDR H3 #5 ARLPLGLQVGFDY
SEQ ID NO: 166 CDR H3 #6 ARVRYSYDLNFDY
SEQ ID NO: 167 CDR H3 #7 ARDDYSDFGFAY
SEQ ID NO: 168 CDR H3 #8 AREANWDDVDY
SEQ ID NO: 169 CDR H3 #9 ARNYYSSSYDGYFDY
SEQ ID NO: 170 CDR H3 #10 ARSGLRDFDY
SEQ ID NO: 171 CDR H3 #11 ASVSSYGNYFDY
SEQ ID NO: 172 CDR H3 #12 AREAKLGRDYFDY
SEQ ID NO: 173 CDR H3 #13 ARFPYDFDGYFDV
SEQ ID NO: 174 CDR H3 #14 ARNYYGSTYDGYFDY
SEQ ID NO: 175 CDR H3 #15 ARGMEGAMDY
SEQ ID NO: 176 CDR H3 #16 ARSYYGRSGYAMDY
SEQ ID NO: 177 CDR H3 #17 ARNEAVVAIFDWYFDV
SEQ ID NO: 178 CDR H3 #18 ARNYYGNNYDGYFDY
SEQ ID NO: 179 CDR H3 #19 ARFPYDYDGYFDV
SEQ ID NO: 180 CDR H3 #20 ARRYYSSGYDGYFDV
SEQ ID NO: 181 CDR H3 #21 ARLNVLYYFDN
SEQ ID NO: 182 CDR H3 #22 ARGGGYYGYDGYWYFDV
SEQ ID NO: 183 CDR H3 #23 ARGANWGDY
SEQ ID NO: 184 CDR H3 #24 ATYYVDY
SEQ ID NO: 185 CDR H3 #25 ARRVYGYDPYAMNY
SEQ ID NO: 186 CDR H3 #26 APHYYGSSYDWYFDV
SEQ ID NO: 187 CDR H3 #27 AREDDSRYWYFDV
SEQ ID NO: 188 CDR H3 #28 AREGWLDAMDY
SEQ ID NO: 189 CDR H3 #29 APRLGLRAY
SEQ ID NO: 190 CDR H3 #30 ARMGGTGYFDV
SEQ ID NO: 191 CDR H3 #31 ARGLDVMDY
SEQ ID NO: 192 CDR L1 #1 SGDKLGDKYVS
SEQ ID NO: 193 CDR L1 #2 FGDKLGHKYVS
SEQ ID NO: 194 CDR L1 #3 SGDKLGDKFTS
SEQ ID NO: 195 CDR L1 #4 QGDSLRNYYAS
SEQ ID NO: 196 CDR L1 #5 QGDSLRSYYAS
SEQ ID NO: 197 CDR L1 #6 SGDKLGDKYAS
SEQ ID NO: 198 CDR L1 #7 NIYSFLAWY
SEQ ID NO: 199 CDR L1 #8 SVSYMYWY
SEQ ID NO: 200 CDR L1 #9 SIVHSNGNTYLEWY
SEQ ID NO: 201 CDR L1 #10 SVDNYGISFMHWY
SEQ ID NO: 202 CDR L1 #11 SISDYLHWY
SEQ ID NO: 203 CDR L1 #12 SVSIHASHLLHWYQ
SEQ ID NO: 204 CDR L1 #13 DVSTAVAWC
SEQ ID NO: 205 CDR L1 #14 DVGTAVAWY
SEQ ID NO: 206 CDR L1 #15 DVSTAVAWY
SEQ ID NO: 207 CDR L1 #16 NVGTYVSWY
SEQ ID NO: 208 CDR L1 #17 DISNYLNWY
SEQ ID NO: 209 CDR L1 #18 NVDNYGISFMHWY
SEQ ID NO: 210 CDR L1 #19 SVSTSGYTYMHWY
SEQ ID NO: 211 CDR L1 #20 SLLHSNGITYLYWY
SEQ ID NO: 212 CDR L1 #21 SLVHSNGNTYLHWF
SEQ ID NO: 213 CDR L1 #22 SLLDSDGKTYLNWF
SEQ ID NO: 214 CDR L1 #23 NVGTAVAWY
SEQ ID NO: 215 CDR L1 #24 DINSYLSWF
SEQ ID NO: 216 CDR L1 #25 NVGPYVSWY
SEQ ID NO: 217 CDR L1 #26 RASENIYSYLA
SEQ ID NO: 218 CDR L2 #1 QDTKRPS
SEQ ID NO: 219 CDR L2 #2 QDSKRPS
SEQ ID NO: 220 CDR L2 #3 QDNKRPS
SEQ ID NO: 221 CDR L2 #4 EDSKRPS
SEQ ID NO: 222 CDR L2 #5 GKNNRPS
SEQ ID NO: 223 CDR L2 #6 QDTKRAS
SEQ ID NO: 224 CDR L2 #7 KTLAEGVPS
SEQ ID NO: 225 CDR L2 #8 NLASGVPV
SEQ ID NO: 226 CDR L2 #9 SNRFSGVPD
SEQ ID NO: 227 CDR L2 #10 NLESGIPA
SEQ ID NO: 228 CDR L2 #11 QSISGIPS
SEQ ID NO: 229 CDR L2 #12 NLESGVPA
SEQ ID NO: 230 CDR L2 #13 YRYTGVPD
SEQ ID NO: 231 CDR L2 #14 TRHTGVPD
SEQ ID NO: 232 CDR L2 #15 NRYTGVPD
SEQ ID NO: 233 CDR L2 #16 SRLHSGVPS
SEQ ID NO: 234 CDR L2 #17 NLEYGIPA
SEQ ID NO: 235 CDR L2 #18 NLASGVPD
SEQ ID NO: 236 CDR L2 #19 SKLDSGVPD
SEQ ID NO: 237 CDR L2 #20 TLASGVPA
SEQ ID NO: 238 CDR L2 #21 NRLVDGVPS
SEQ ID NO: 239 CDR L2 #22 NRFTGVPD
SEQ ID NO: 240 CDR L2 #23 NAKILAE
SEQ ID NO: 241 CDR L3 #1 QAWDSGTAI
SEQ ID NO: 242 CDR L3 #2 QAWDSSTVV
SEQ ID NO: 243 CDR L3 #3 QVWDSSSDHWV
SEQ ID NO: 244 CDR L3 #4 NSRDSSGNHPVV
SEQ ID NO: 245 CDR L3 #5 NSRDSSGNHLV
SEQ ID NO: 246 CDR L3 #6 NSRDSSGNHVI
SEQ ID NO: 247 CDR L3 #7 QAFDSSAAHFV
SEQ ID NO: 248 CDR L3 #8 QTWDSSTAV
SEQ ID NO: 249 CDR L3 #9 QHHYGIPPT
SEQ ID NO: 250 CDR L3 #10 QQWSSYPLT
SEQ ID NO: 251 CDR L3 #11 FQGSHVPYT
SEQ ID NO: 252 CDR L3 #12 QQSNKDPRT
SEQ ID NO: 253 CDR L3 #13 QNGHSFPLT
SEQ ID NO: 254 CDR L3 #14 QQSIEDPWT
SEQ ID NO: 255 CDR L3 #15 QHYSTPYT
SEQ ID NO: 256 CDR L3 #16 QQYSSYPLT
SEQ ID NO: 257 CDR L3 #17 QQHYSTPPT
SEQ ID NO: 258 CDR L3 #18 GQSYSYPPFT
SEQ ID NO: 259 CDR L3 #19 QQGNTLPWT
SEQ ID NO: 260 CDR L3 #20 HQFSSYPLT
SEQ ID NO: 261 CDR L3 #21 QQSNKDPLT
SEQ ID NO: 262 CDR L3 #22 QHSRELPLT
SEQ ID NO: 263 CDR L3 #23 AQNLELPWT
SEQ ID NO: 264 CDR L3 #24 SQSTHVPPT
SEQ ID NO: 265 CDR L3 #25 WQGTHFPQT
SEQ ID NO: 266 CDR L3 #26 QQWSSNPYT
SEQ ID NO: 267 CDR L3 #27 QQYSSYPYT
SEQ ID NO: 268 CDR L3 #28 QQYSSYTWT
SEQ ID NO: 269 CDR L3 #29 LQYDEFPYT
SEQ ID NO: 270 CDR L3 #30 GQSYSYPFT
SEQ ID NO: 271 CDR L3 #31 QHHYVSPRT
SEQ ID NO: 272 Linker (G4S)3 GGGGSGGGGGGGGS
SEQ ID NO: 273 CD28 Co-stimulatory domain
IEVMYPPPYLDNEKSNGTIIHVKGKHLCPSPLFPGPSKPFWVLVVVGGVLACYSLLVTVAFIIFWVRSKRSRLLHSDYMN
MTPRRPGPTRKHYQPYAPPRDFAAY
SEQ ID NO: 274 CD3 zeta Signaling Domain
MKWKALFTAAILQAQLPITEAQSFGLLDPKLCYLLDGILF IYGVILTALF LRVKFSRSADAPAYQQGQNQ LYNELNLGRR
EEYDVLDKRR GRDPEMGGKP QRRKNPQEGL YNELQKDKMAEAYSEIGMKG ERRRGKGHDG LYQGLSTATK
DTYDALHMQA LPPR
SEQ ID NO: 275 Human CD8α transmembrane domain
IYIWAPLAGTCGVLLLSLVIT
SEQ ID NO: 276 Human CD8α transmembrane domain
IYIWAPLAGTCGVLLLSLVITLYCNHRN
SEQ ID NO: 277 Human CD28 transmembrane domain
FWVLVVVGGVLACYSLLVTVAFIIFWV
SEQ ID NO: 278 OX40 Co-stimulatory Domain
ALYLLRRDQRLPPDAHKPPGGGSFRTPIQEEQADAHSTLAKI
SEQ ID NO: 279: 4-1BB Co-stimulatory Domain
KRGRKKLLYIFKQPFMRPVQTTQEEDGCSCRFPEEEEGGCEL
SEQ ID NO: 280 T2A cleavage sequence
GSGEGRGSLLTCGDVEENPGP
SEQ ID NO: 281 PZA cleavage sequence
GSGATNFSLLKQAGDVEENPGP
SEQ ID NO: 282 E2A cleavage sequence
GSGQCTNYALLKLAGDVESNPGP
SEQ ID NO: 283 F2A cleavage sequence
GSGVKQTLNFDLLKLAGDVESNPGP
SEQ ID NO: 284 Tbx21 Intracellular Signaling Domain
MGIVEPGCGDMITGTEPMPGSDEGRAPGADPQHRYFYPEPGAQDADERRGGGSLGSPYPGGALVPAPPSRFLGAYA
YPPRPQAAGFPGAGESFPPPADAEGYQPGEGYAAPDPRAGLYPGPREDYALPAGLEVSGKLRVALNNHLLWSKFNQH
QTEMIITKQGRRMFPFLSFTVAGLEPTSHYRMFVDVVLVDQHHWRYQSGKWVQCGKAEGSMPGNRLYVHPDSPNT
GAHWMRQEVSFGKLKLTNNKGASNNVTQMIVLQSLHKYQPRLHIVEVNDGEPEAACNASNTHIFTFQETQFIAVTAY
QNAEITQLKIDNNPFAKGFRENFESMYTSVDTSIPSPPGPNCQFLGGDHYSPLLPNQYPVPSRFYPDLPGQAKDVVPQA
YWLGAPRDHSYEAEFRAVSMKPAFLPSAPGPTMSYYRGQEVLAPGAGWPVAPQYPPKMGPASWFRPMRTLPMEP
GPGGSEGRGPEDQGPPLVWTEIAPIRPESSDSGLGEGDSKRRRVSPYPSSGDSSSPAGAPSPFDKEAEG QFYNYFPN
SEQ ID NO: 285 E2S-VP64 Intracellular Signaling Domain
MAQAALEPGEKPYACPECGKSFSTSGSLVRHQRTHTGEKPYKCPECGKSFSRNDALTEHQRTHTGEKPYKCPECGKSFS
SKKHLAEHQRTHTGEKPYACPECGKSFSTSGELVRHQRTHTGEKPYKCPECGKSFSRSDKLVRHQRTHTGEKPYKCPEC
GKSFSRSDHLTEHQRTHTGKKTSGQAGQASPKKKRKVGRADALDDFDLDMLGSDALDDFDLDMLGSDALDDFDLDM
LGSDALDDFDLDMLINYPYDVPDYAS
SEQ ID NO: 286 GAL4-VP64 Intracellular Signaling Domain
MKLLSSIEQACDICRLKKLKCSKEKPKCAKCLKNNWECRYSPKTKRSPLTRAHLTEVESRLERLEQLFLLIFPREDLDMILK
MDSLQDIKALLTGLFVQDNVNKDAVTDRLASVETDMPLTLRQHRISATSSSEESSNKGQRQLTVSAAAGGSGGSGGSD
ALDDFDLDMLGSDALDD FDLDMLGSDALDDFDLDMLGSDALDDFDLDMLGS
SEQ ID NO: 287 CD19 scFv Variable Heavy Domain
LKPREWKLVESGGGLVQPGGSLKLSCAASGFDFSRYWMSWVRQAPGKGLEWIGEINLDSSTINYTPSLKDKFIISRDNA
KNTLYLQMSKVRSEDTALYYCARRYDAMDYWGQGTSVTVSSAKTTAPSVYPLAPVCGDTTGSSVTLGCLVKASQ
SEQ ID NO: 288 CD19 scFv Variable Light Domain
ASDIWLTQSPASLAVSLGQRATISCRASESVDDYGISFMNWFQQKPGQ
PPKLLIYAAPNQGSGVPARFSGSGSGTDFSLNIHPMEEDDTAMYFCQQSKDVRWRHQAGDQTG
SEQ ID NO: 289 CXCR5
MNYPLTLEMDLENLEDLFWELDRLDNYNDTSLVENHLCPATEGPLMASFKAVFVPVAYSLIFLLGVIGNVLVLVILERHR
QTRSSTETFLFHLAVADLLLVFILPFAVAEGSVGWVLGTFLCKTVIALHKVNFYCSSLLLACIAVDRYLAIVHAVHAYRHRR
LLSIHITCGTIWLVGFLLALPEILFAKVSQGHHNNSLPRCTFSQENQAETHAWFTSRFLYHVAGFLLPMLVMGWCYVGV
VHRLRQAQRRPQRQKAVRVAILVTSIFFLCWSPYHIVIFLDTLARLKAVDNTCKLNGSLPVAITMCEFLGLAHCCLNPML
YTFAGVKFRSDLSRLLTKLGCTGPASLCQLFPSWRRSSLSESENATSLTTF
SEQ ID NO: 290 CXCR7
MYSIICFVGLLGNGLVVLTYIYFKRLKTMTDTYLLNLAVADILFLLTLPFWAYSAAKSWVFGVHFCKLIFAIYKMSFFSGML
LLLCISIDRYVAIVQAVSAHRHRARVLLISKLSCVGIWILATVLSIPELLYSDLQRSSSEQAMRCSLITEHVEAFITIQVAQMV
IGFLVPLLAMSFCYLVIIRTLLQARNFERNKAIKVIIAVVVVFIVFQLPYNGVVLAQTVANFNITSSTCELSKQLNIAYDVTYS
LACVRCCVNPFLYAFIGVKFRNDLFKLFKDLGCLSQEQLRQWSSCRHIRRSSMSVEAETTTTFSP
*SEQ ID NOs: 39-114 show both underlined and bolded amino acids. The underlined amino acids represent CDRs 1-3, respectively, in either the Variable Heavy Chain Region (SEQ ID NOs: 39-76) or the Variable Light Chain Region (SEQ IDs: 77-114) annotated by the IMGT method. The bolded amino acids represent CDRs 1-3, respectively, in either the Variable Heavy Chain Region (SEQ ID NOs: 39-76) or the Variable Light Chain Region (SEQ ID NOs: 77-114) annotated by the Kabat method.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for producing an immuno-activatable cell, wherein the method comprises introducing into the cell:
(a) a first nucleic acid sequence, wherein the first nucleic acid sequence encodes a first chimeric antigen receptor polypeptide, wherein the first chimeric antigen receptor polypeptide comprises a first extracellular domain, a first transmembrane domain, and a first intracellular domain, wherein the first extracellular domain comprises a first antigen binding domain capable of binding to a first antigen on a CD11c⁺Tbet⁺ B cell, wherein the first transmembrane domain comprises a first CD8α transmembrane domain, wherein the first intracellular domain comprises a cytoplasmic signaling domain, and wherein the sequence encoding the first chimeric antigen receptor polypeptide is operably linked to a first promoter; and
(b) a second nucleic acid sequence, wherein the second nucleic acid sequence encodes a second chimeric antigen receptor polypeptide, wherein the second chimeric antigen receptor polypeptide comprises a second extracellular domain, a second transmembrane domain, and a second intracellular domain, wherein the second extracellular domain comprises a second antigen binding domain capable of binding to a second antigen on the CD11c⁺Tbet⁺ B cell, wherein the second transmembrane domain comprises a second CD8α transmembrane domain, wherein the intracellular domain comprises a co-stimulatory domain, and wherein the sequence encoding the second chimeric antigen receptor polypeptide is operably linked to a second promoter.

2. The method of claim 1, wherein the first antigen binding domain is an antibody or an antigen binding fragment.

3. (canceled)

4. The method of claim 1, wherein the first antigen is a B cell receptor.

5. The method of claim 4, wherein the B cell receptor is CD19, CD20, or CD45R.

6. (canceled)

7. The method of claim 1, wherein the second antigen binding domain is an antibody or an antigen binding fragment.

8.-9. (canceled)

10. The method of claim 5, wherein the first antigen binding domain comprises an scFv comprising a sequence at least 90% identical to one of SEQ ID NOs: 1-10.

11. The method of claim 10, wherein the first antigen binding domain comprises one of the following:
(a) a heavy chain variable domain comprising SEQ ID NO: 39 and a light chain variable domain comprising SEQ ID NO: 77;
(b) a heavy chain variable domain comprising SEQ ID NO: 40 and a light chain variable domain comprising SEQ ID NO: 78;
(c) a heavy chain variable domain comprising SEQ ID NO: 41 and a light chain variable domain comprising SEQ ID NO: 79;
(d) a heavy chain variable domain comprising SEQ ID NO: 42 and a light chain variable domain comprising SEQ ID NO: 80;
(e) a heavy chain variable domain comprising SEQ ID NO: 43 and a light chain variable domain comprising SEQ ID NO: 81;
(f) a heavy chain variable domain comprising SEQ ID NO: 44 and a light chain variable domain comprising SEQ ID NO: 82;
(g) a heavy chain variable domain comprising SEQ ID NO: 45 and a light chain variable domain comprising SEQ ID NO: 83;
(h) a heavy chain variable domain comprising SEQ ID NO: 46 and a light chain variable domain comprising SEQ ID NO: 84;
(i) a heavy chain variable domain comprising SEQ ID NO: 47 and a light chain variable domain comprising SEQ ID NO: 85; or
(j) a heavy chain variable domain comprising SEQ ID NO: 48 and a light chain variable domain comprising SEQ ID NO: 86.

12. The method of claim 7, wherein the second antigen is CD11c.

13. The method of claim 12, wherein the second antigen binding domain comprises an scFv comprising a sequence at least 90% identical to SEQ ID NO: 37 or SEQ ID NO: 38.

14. The method of claim 13, wherein the second antigen binding domain comprises either
(a) a heavy chain variable domain comprising SEQ ID NO: 75 and a light chain variable domain comprising SEQ ID NO: 113; or
(b) a heavy chain variable domain comprising SEQ ID NO: 76 and a light chain variable domain comprising SEQ ID NO: 114.

15. The method of claim 1, wherein the cytoplasmic signaling domain is a CD3zeta, CD3epsilon, CD3delta, TCRzeta, FcR gamma, FcR beta, CD5, CD22, CD79a, CD79b, or CD66d domain.

16. The method of claim 1, wherein the signaling domain is a CD28, 4-1BB, CD97, CD11a-CD18, CD2, CD27, ICOS, CD154, CD5, or OX40 signaling domain.

17. The method of claim 1, wherein the first and second CD8α transmembrane domains further comprise a CD8α hinge domain and a CD8α stalk domain.

18. The method of claim 1, further comprising introducing into the cell a third nucleic acid sequence, wherein the third nucleic acid sequence encodes a first chemokine receptor polypeptide.

19. The method of claim 18, further comprising introducing into the cell a fourth nucleic acid sequence, wherein the fourth nucleic acid sequence encodes a second chemokine receptor polypeptide.

20. (canceled)

21. The method of claim 18, wherein said first or second chemokine receptor polypeptide is CXCR5 or CCR7.

22. (canceled)

23. The method of claim 1, wherein the immuno-activatable cell is an immune cell selected from the group consisting of a T cell, a B cell, a monocyte, a natural killer cell, a dendritic cell, a macrophage, a regulatory T cell, a helper T cell, and a cytotoxic T cell.

24. An immuno-activatable cell produced by the method of claim 1.

25. A method for treating a mammal having an autoimmune disease, wherein the method comprises administering to the mammal identified as having an autoimmune disease an effective amount of the immuno-activatable cell of claim 24, wherein the immuno-activatable cell expresses a first chimeric antigen receptor polypeptide having a first antigen binding domain that binds a first antigen on a CD11c⁺Tbet⁺ B cell with low affinity, wherein the binding activates the immuno-activatable cell, and wherein the immuno-activatable cell expresses a second chimeric antigen receptor polypeptide having a second antigen binding domain that binds a second antigen on a CD11c⁺Tbet⁺ B cell and stimulates the immuno-activatable cell.

26. (canceled)

27. The method of claim 25, wherein the autoimmune disease results from production of autoantibodies by age-associated B cells.

28. The method of claim 25, wherein the autoimmune disease is lupus, rheumatoid arthritis, multiple sclerosis, insulin dependent diabetes mellitis, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, Crohn's disease, Celiac disease, or polyarteritis nodosa.

29. (canceled)

30. A method for producing a T cell targeting age-associated B cells, wherein the method comprises introducing into the T cell:
i. a first nucleic acid sequence, wherein the first nucleic acid sequence encodes a first chimeric antigen receptor polypeptide, wherein the first chimeric antigen receptor polypeptide comprises an extracellular domain, a transmembrane domain and an intracellular domain, wherein the extracellular domain comprises a first antigen binding domain capable of binding to CD19 antigen on the surface of a CD11c⁺Tbet⁺ B cell, wherein the first transmembrane domain comprises a first CD8α transmembrane domain, wherein the first intracellular domain comprises CD3zeta domain, and wherein the sequence encoding the first chimeric antigen receptor polypeptide is operably linked to a first promoter; and
ii. a second nucleic acid sequence, wherein the second nucleic acid sequence encodes a second chimeric antigen receptor polypeptide, wherein the second chimeric antigen receptor polypeptide comprises a second extracellular domain, a second transmembrane domain and a second intracellular domain, wherein the second extracellular domain comprises a second antigen binding domain capable of binding to a CD11c antigen on the surface of a CD11c⁺Tbet⁺ B cell, wherein the second transmembrane domain comprises a second CD8α transmembrane domain, wherein the second intracellular domain comprises a CD28 signaling domain, and wherein the sequence encoding the second chimeric antigen receptor polypeptide is operably linked to a second promoter.

31.-35. (canceled)

36. A transformed T cell produced by the method of claim 30.

37. A method for treating a mammal having an autoimmune disease, wherein the method comprises administering to the mammal an effective amount of the transformed T cell of claim 36, wherein the transformed T cell expresses a first chimeric antigen receptor polypeptide having a first antigen binding domain that binds a CD19 antigen on a CD11c⁺Tbet⁺ B cell with low affinity, wherein the binding activates the T cell, and wherein the T cell expresses a second chimeric antigen receptor polypeptide having a second antigen binding domain that binds a CD11c antigen on a CD11c⁺Tbet⁺ B cell and stimulates the T cell, thereby treating an autoimmune disease in the mammal.

38.-41. (canceled)

42. A chimeric antigen receptor polypeptide comprising an extracellular domain, a transmembrane domain, and an intracellular domain, wherein the extracellular domain comprises an antigen binding domain capable of binding to an antigen on a CD11c⁺Tbet⁺ B cell, wherein the transmembrane domain comprises a CD8α transmembrane domain, and wherein the intracellular domain comprises a cytoplasmic signaling domain.

43.-50. (canceled)

51. A chimeric antigen receptor polypeptide comprising an extracellular domain, a transmembrane domain, and an intracellular domain, wherein the extracellular domain comprises an antigen binding domain capable of binding to an antigen on a CD11c⁺Tbet⁺ B cell, wherein the transmembrane domain comprises a CD8α transmembrane domain, and wherein the intracellular domain comprises a co-stimulatory domain.

52.-62. (canceled)

63. An isolated nucleic acid encoding the chimeric antigen receptor polypeptide of claim 42.

64. A vector comprising the nucleic acid of claim 63.

65. A cell comprising the nucleic acid of claim 63.

66. The cell of claim 65, wherein the cell is an immune cell selected from the group consisting of a T cell, a B cell, a monocyte, a natural killer cell, a dendritic cell, a macrophage, a regulatory T cell, a helper T cell, and a cytotoxic T cell.
